(19) United States
(12) Patent Application Publication
(10) Pub. No.: US 2022/0362404 A1
(43) Pub. Date: Nov. 17, 2022

(54) MODULATION OF CELLULAR VIABILITY

(71) Applicant: Macquarie University, North Ryde (AU)

(72) Inventors: Stephanie Rayner, North Ryde (AU); Albert Lee, North Ryde (AU); Jennilee Davidson, North Ryde (AU); Roger Chung, North Ryde (AU)

(21) Appl. No.: 17/754,597

(22) PCT Filed: Oct. 21, 2020
(86) PCT No.: PCT/AU2020/051133

§ 371 Date: Apr. 6, 2022

(30) Foreign Application Priority Data
Oct. 21, 2019 (AU) 2019903956

(51) Int. Cl. A61K 48/00

(52) U.S. Cl. CPC A61K 48/005

(57) ABSTRACT

Provided herein are methods for enhancing survival of a neuron, for inhibiting degeneration of a neuron, and for inhibiting abnormal protein accumulation in a neuron, optionally a motor neuron, comprising, consisting or consisting essentially of increasing the level of cyclin F in the neuron regardless of the neuron's level or activity of endogenous cyclin F. Optionally the neuron is in a subject with a neurodegenerative condition or at risk of developing a neurodegenerative condition, typically a neurodegenerative condition associated with a neuronal TDP-43 proteinopathy.

### FIELD

This disclosure relates generally to neurodegenerative conditions. More particularly, the present disclosure relates to compositions and methods for enhancing motor neuron survival, inhibiting motor neuron degeneration and treating neurodegenerative conditions through increasing the level of cyclin F in a motor neuron regardless of the motor neuron's level or activity of endogenous cyclin F.

Certain bibliographic references referred to by author and year of publication in the present specification are listed at the end of the description.

### BACKGROUND

Amyotrophic lateral sclerosis (ALS) is the most common form of motor neuron disease (MND) and refers to the selective degeneration of upper and lower motor neurons of the brain and spinal cord, respectively. ALS and frontotemporal dementia (FTD) sit within a spectrum of disease, with 15% of ALS patients also displaying symptoms of FTD, the second most common form of early-onset dementia. The etiology of ALS and FTD remain poorly understood, however most ALS patients and more than half of all FTD patients share common histopathological features. Post-mortem analysis of ALS-patient brain and spinal cord tissue frequently reveal the presence of tau-negative, ubiquitin-positive aggregates which appear as round or skein-like inclusions, most commonly in the cytoplasm of affected neurons and glia. These inclusions are speckled with ubiquitin, sqstm1, ubiquilin 1 and ubiquilin 2—proteins that are all involved in ubiquitin-mediated protein turnover, suggesting that defective proteasomal clearance is a contributing factor in ALS/FTD pathogenesis. In 2006, the major component of these inclusions was identified as Transactive Response DNA binding protein of 43 kDa (TDP-43), a predominantly nuclear protein found to translocate from the nucleus to the cytoplasm in cases of ALS/FTD. Characterization of sarkosyl-insoluble fractions from patient brain lysates reveals a striking shift in the biochemical profile of TDP-43. In patient lysates, TDP-43 is poly-ubiquitylated, hyper-phosphorylated and cleaved at the C-terminus. TDP-43 proteinopathy has now been identified in over 98% of ALS cases and in over 50% of FTD cases regardless of familial or sporadic origin, making TDP-43-positive aggregates or inclusions a hallmark feature of the disease.

Contrary to its insoluble, aggregated pathological form, soluble TDP-43 (sTDP-43) is required for normal cellular function. In this regard, it participates in several mechanisms of mRNA metabolism like pre-mRNA splicing, mRNA stability, mRNA transport and miRNA processing and is necessary for neuronal viability. In normal conditions, the subcellular localization of sTDP-43 is predominantly nuclear, but the presence of a nuclear localization sequence (NLS) and a nuclear export sequence in the N-terminus of the protein allow sTDP-43 to shuttle between the nucleus and cytoplasm. sTDP-43 is also known to regulate mRNAs involved in the development of neurons and embryos and is expressed throughout CNS development into adulthood. As such, it is understood that sTDP-43 is an essential RNA binding protein and alterations in its ability to carry out its cellular roles are toxic to neuronal cells.

Familial ALS (fALS) mutations account for 5-10% of all ALS cases whilst the remaining cases have no clear cause (sporadic ALS; sALS). Although familial gene mutations account for the minority of ALS cases, they have provided invaluable insight into the mechanisms underlying disease. Accordingly, mutations have been identified in numerous genes including SOD1, VCP, TARDBP, FUS, OPTN, SQSTM1, UBQLN2, MATR3 and TBK1. Interestingly mutations in TARDBP, the gene encoding TDP-43, are found in only ˜4% of fALS patients and around 1% of sALS cases.

There is strong evidence that the subcellular location of TDP-43 within motor neurons is central to the neurodegeneration phenotype. For example, abnormal cytoplasmic accumulation (insoluble aggregates) of TDP-43 are the pathological hallmark of ALS (98% of cases) and FTD (>50%). In 2015, a transgenic mouse was generated with inducible overexpression of a human TDP-43 variant that specifically mislocalizes to the cytoplasm (variant is termed dNLS-TDP-43). When overexpressed, dNLS-TDP43 mice develop rapid ALS-like phenotype resulting in motor paralysis and death. This dNLS-TDP-43 mouse represents an experimental model of sporadic ALS/FTD, since it specifically causes cytoplasm-mislocalized TDP-43 reminiscent of sporadic disease.

ALS/FTD-associated mutations have been identified in CCNF, which occur at similar frequencies to those found in TARDBP. CCNF encodes cyclin F, the ligand-binding component of the multi-protein Skp1-Cul1-F-Box (SCF^{Cyclin F}) E3 ligase. Within this SCF complex, cyclin F (the F-box protein) is responsible for recruiting and positioning substrates for poly-ubiquitylation, which is followed by their proteasomal degradation. To date, cyclin F activity has been heavily associated with cell cycle progression and DNA damage as it mediates ubiquitylation of ribonucleoside-diphosphate reductase subunit M2 (RRM2), nucleolar and spindle-associated protein 1 (NuSAP), centriolar coiled-coil protein of 110 kDa (CP110), cell division control protein 6 homolog (CDC6), histone RNA hairpin-binding protein (SLBP) exonuclease 1 (exo1) and fizzy-related protein homolog (Fzr1). Cyclin F is also known to bind and alter the mitotic transcriptional program of myb-related protein B (B-Myb). Importantly, all of these studies report nuclear localization of cyclin F, consistent with its function as a cell cycle regulatory protein.

In previous work by the present inventors, it was found (1) that TDP-43 is an interaction partner and substrate of the SCF^{Cyclin F} complex, (2) that a deficiency in cyclin F leads to an accumulation of TDP-43 in motor neurons and (3) that a subset of patients with a neurodegenerative condition have an abnormally low level or activity of cyclin F in motor neurons. Based on these findings, the present inventors disclosed in WO 2018/081878 that increasing cyclin F levels in motor neurons in this subset of patients with a neurodegenerative condition reduces abnormal accumulation of proteins to thereby enhance motor neuron survival.

The present inventors have also identified a serine to glycine substitution at position 621 (S621G) of cyclin F in a multi-generational Australian family with ALS/FTD, which causes overactive ubiquitylation of TDP-43 and other substrates (Lee et al., 2017). Taken together with the findings disclosed in WO 2018/081878, the present inventors postulated that cyclin F activity is tightly regulated for the maintenance of appropriate activity of ubiquitylation-dependent protein degradative pathways, and that dysregulation that leads to low levels or overactive activity of cyclin F impairs these pathways and triggers neurodegenerative diseases such as ALS and FTD.

### SUMMARY

The present disclosure arises from the discovery that it is possible to enhance survival of neurons, including motor neurons, which have normal levels of endogenous cyclin F by supplementing the neurons with additional cyclin F. The present inventors have found unexpectedly that this supplementation reduces the level of insoluble TDP-43 (insTDP-43) without significantly reducing sTDP-43, thereby selectively targeting the pathological form of TDP-43 whilst permitting its soluble form to carry out its normal cellular functions. This finding was surprising because it was assumed that expression of additional cyclin F would be directed to the nucleus, in line with what was known about its role in cell division, and that this localization would result in depletion of nuclear TDP-43 (sTDP-43) and a consequential ALS-like phenotype (Wu et al., 2012). Without wishing to be bound by any particular theory, the present inventors propose that in contrast to other cell types, expression of cyclin F in neurons, including motor neurons, is localized to the cytoplasm, which permits selective targeting and sequestration of pathological insTDP-43.

The present inventors have also found that cyclin F directly binds and mediates the poly-ubiquitylation of insTDP-43 for entry into the ubiquitin-proteasome proteolytic pathway, and that this occurs via an atypical interaction independent of the known substrate recognition motif in cyclin F (MRYIL) and the binding motif in substrates (R-X-L). Without wishing to be bound by any particular theory, it is believed that an atypical binding motif present in cyclin F selectively targets insTDP-43 for clearance and provides a biological rationale as to why cyclin F is able to perform distinct and discrete functions in two markedly different cell types—dividing cells, and non-dividing neurons.

These findings have been reduced to practice in methods for enhancing neuron survival, including motor neuron survival, regardless of the level or activity of endogenous cyclin F in a neuron, and/or in which the neuron does not have a reduced level or activity of endogenous cyclin F relative to a control, for treating neurodegenerative diseases, including familial and sporadic neurodegenerative diseases that are suitably associated with a TDP-43 proteinopathy, as described hereafter.

Accordingly, in one aspect, the present disclosure provides methods for enhancing survival of a neuron (e.g., a motor neuron), suitably in a subject with a neurodegenerative condition or at risk of developing a neurodegenerative condition. These methods generally comprise, consist or consist essentially of increasing the level of cyclin F in the neuron regardless of the neuron's level or activity of endogenous cyclin F, thereby enhancing survival of the neuron.

Another aspect of the present disclosure provides methods for inhibiting degeneration of a neuron (e.g., a motor neuron), suitably in a subject with a neurodegenerative condition or at risk of developing a neurodegenerative condition. These methods generally comprise, consist or consist essentially of increasing the level of cyclin F in the neuron regardless of the neuron's level or activity of endogenous cyclin F, thereby inhibiting degeneration of the neuron.

In yet another aspect, the present disclosure provides methods for inhibiting abnormal protein accumulation in a neuron (e.g., a motor neuron), suitably in a subject with a neurodegenerative condition or at risk of developing a neurodegenerative condition. These methods generally comprise, consist or consist essentially of increasing the level of cyclin F in the neuron regardless of the neuron's level or activity of endogenous cyclin F, thereby inhibiting abnormal protein accumulation in the neuron. Suitably, the abnormal protein accumulation comprises abnormal accumulation of proteins (e.g., proteins that are susceptible to protein accumulation or aggregation such as TDP-43).

In a related aspect, the present disclosure provides methods for inhibiting aggregated or insoluble TDP-43 accumulation in a neuron (e.g., a motor neuron), suitably in a subject with a neurodegenerative condition or at risk of developing a neurodegenerative condition. These methods generally comprise, consist or consist essentially of increasing the level of cyclin F in the neuron regardless of the neuron's level or activity of endogenous cyclin F, thereby inhibiting aggregated or insoluble TDP-43 accumulation in the neuron.

Still another aspect of the present disclosure provides methods for treating a subject with a neurodegenerative condition or at risk of developing a neurodegenerative condition. These methods generally comprise, consist or consist essentially of increasing the level of cyclin F in a neuron (e.g., a motor neuron) of the subject regardless of the neuron's level or activity of endogenous cyclin F.

In any of the above aspects or embodiments, the methods suitably comprise contacting the neuron (e.g., a motor neuron) with an agent that increases the level of cyclin F in the neuron. In specific embodiments, the methods comprise administering an effective amount of the agent to the subject. In some embodiments, the agent comprises a construct that comprises a nucleotide sequence encoding cyclin F in operable connection with a promoter that is operable in the neuron (e.g., a motor neuron). In illustrative examples of this type, the construct is contained in a delivery vehicle (e.g., a viral vector such as an adeno-associated virus (AAV) vector, or a non-viral vector). In specific embodiments, the methods comprise administering an effective amount of the construct to the subject.

In any of the above aspects or embodiments, the methods suitably comprise overexpressing a coding sequence for cyclin F in the neuron (e.g., a motor neuron).

In any of the above aspects or embodiments, the neuron (e.g., a motor neuron) may have a normal level or activity of endogenous cyclin F relative to a control.

In any of the above aspects or embodiments, the neuron (e.g., a motor neuron) may not have a reduced level or activity of endogenous cyclin F relative to a control.

In any of the above aspects or embodiments, the methods suitably lack a step of detecting a reduced level or activity of endogenous cyclin F in the neuron (e.g., a motor neuron) relative to a control, prior to increasing the level of cyclin F in the neuron.

In any of the above aspects or embodiments, the methods may comprise a step of detecting a level or activity of endogenous cyclin F in the neuron (e.g., a motor neuron) relative to a control, which is not a reduced level or activity of endogenous cyclin F in the neuron relative to the control, prior to increasing the level of cyclin F in the neuron.

In any of the above aspects or embodiments, the methods may comprise a step of detecting a normal level or activity of endogenous cyclin F in the neuron (e.g., a motor neuron) relative to a control, prior to increasing the level of cyclin F in the neuron.

In any of the above aspects or embodiments, the subject suitably has a neurodegenerative condition or is at risk of developing a neurodegenerative condition, wherein the neurodegenerative condition is associated with a neuronal TDP-43 proteinopathy. In representative examples of this type, the subject may have a familial neurodegenerative condition (e.g., familial ALS, familial FTD, familial Alzheimer's Disease (AD), etc.) or a sporadic neurodegenerative condition (e.g., sporadic ALS, sporadic FTD, sporadic AD, etc.).

Another aspect of the present disclosure relates to use of an agent that increases the level of cyclin F in a neuron (e.g., a motor neuron) in the manufacture of a medicament for treating or inhibiting the development of a neurodegenerative condition associated with a neuronal TDP-43 proteinopathy, regardless of the neuron's level or activity of endogenous cyclin F.

In a related aspect, the present disclosure provides a kit comprising an agent that increases the level of cyclin F in a neuron (e.g., a motor neuron) for use in a method for treating or inhibiting the development of a neurodegenerative condition associated with a neuronal TDP-43 proteinopathy, regardless of the neuron's level or activity of endogenous cyclin F. In some embodiments, the kit further comprises instructional material for performing the method.

### BRIEF DESCRIPTION OF THE DRAWINGS

FIG. 1 is a photographic and schematic representation showing that cyclin F binds TDP-43. A. HEK293 cells were transfected with cyclin F-flag and TDP-43-HA. After 24 hours, cells were lysed and anti-flag antibodies or an IgG control were used to immunoprecipitate Flag-cyclin F. Eluates were analyzed by immunoblotting using antibodies recognizing cyclin F and TDP-43. B. HEK293 cells were transfected with cyclin F-flag and TDP-43-HA. At 24 hours post-transfection, cells were lysed and Flag-cyclin F was immunoprecipitated using antibodies recognizing the Flag tag. Eluates were analyzed by immunoblotting using antibodies recognizing cyclin F and phospho-TDP-43 (S409/410). C. Schematic showing the position of the RxL>AxA mutation within TDP-43. D. Cells were transfected with cyclin F and TDP-43(WT) or TDP-43(RxL>AxA). Cyclin F was immunoprecipitated from cell lysates using antibodies recognizing the Flag tag. Eluates were analyzed using anti-cyclin F and anti-TDP-43. E. Schematic showing the TDP-43 fragments used for immunoprecipitations. F. HEK293 cells were transfected with constructs encoding Flag-cyclin F alongside constructs encoding Myc-tagged N-terminal or C-terminal fragments of TDP-43. After cell lysis, anti-myc antibody was used to immunoprecipitate myc-TDP-43. Eluates were analyzed by immunoblotting using antibodies recognizing cyclin F and myc.

FIG. 2 is a schematic and photographic representation showing that SCF^{cyclin F} directly binds and ubiquitylates TDP-43. A. Schematic showing recombinant proteins used for pull-down studies. B. Recombinant cyclin F and recombinant TDP-43 were incubated together before TDP-43 was enriched by Ni-NTA magnetic beads. Eluates were analyzed by immunoblotting using anti-cyclin F and anti-TDP-43. C. The cyclin domain of cyclin F was made recombinantly and incubated with recombinant TDP-43. After incubation, TDP-43 was pulled down via the His-tag. Eluates were analyzed by immunoblotting using antibodies recognizing GST and TDP-43. D. An in vitro ubiquitylation assay was conducted using immunoprecipitated Flag-cyclin F and recombinant His-TDP-43.

FIG. 3 presents the TDP-43 amino acid sequence, showing that TDP-43 carries a single RxL motif between amino acids 268-270.

FIG. 4 is a schematic and graphical representation showing that full-length TDP-43 directly binds the cyclin domain of cyclin F. A. Microscale thermophoresis (MST) output from interaction between GST alone and TDP-43 shows no clear binding. B. MST output from interaction between GST-cyclin domain and TDP-43 demonstrates protein binding in a concentration dependent manner.

FIG. 5 is a photographic and graphical representation showing that overexpression of cyclin F in the central nervous system of mice leads to the reduction of RIPA-insoluble TDP-43 species. A. Immunoblot showing insTDP-43 species isolated from the motor cortex of mice as well as ponceau S. B. Densitometry of lanes corresponding to TDP-43 normalized to GAPDH. A student's t-test was used to determine whether the densitometry between groups was significantly different. Left hand bar: WT cyclin F; right hand bar: GFP. C. Immunoblot showing insoluble TDP-43. D. Densitometry of lanes corresponding to TDP-43 normalized to ponceau S. Left hand bar: GFP; right hand bar: WT cyclin F. A student's t-test was used to determine whether the densitometry between groups was significantly different. **p<0.01.

FIG. 6 provides graphical representation showing that experimental overexpression of wild type cyclin F (WT) in transgenic zebrafish overexpressing human TDP-43 selectively in motor neurons leads to the reduction in nuclear levels of human TDP-43. Overexpression of cyclin F(LP/AA), which does not form an active ubiquitin ligase, does not lead to a reduction in TDP-43 levels. A. Whole cell fluorescence intensity of human TDP-43 in zebrafish motor neurons after injection of wildtype (WT) human CCNF mRNA or injection of mRNA encoding human cyclin F(LP/AA) variant (WT-IA). B. Cytoplasmic fluorescence intensity of human TDP-43 in zebrafish motor neurons after injection of wildtype (WT) human CCNF mRNA or injection of mRNA encoding human cyclin F(LP/AA) variant (WT-IA). C. Nuclear fluorescence intensity of human TDP-43 in zebrafish motor neurons after injection of wildtype (WT) human CCNF mRNA or injection of mRNA encoding human cyclin F(LP/AA) variant (WT-IA). A student's t-test was used to determine that there is a statistically significant difference between experimental groups. * p<0.05; *** p<0.001; **** p<0.0001.

TABLE A
Brief Description of thc Sequences

| GenBank | | SEQ ID |
|---|---|---|
| Accession No. | SEQUENCE | NO: |
| NM_001323538 | ctgcgcctgcgcgagggctacgcgcgctccggccggggcgcgggcgcgctctcaggcg | 1 |
| | ggctccggcggcagcgacgcgagcgcggcgatggggagcggcggcgtggtccactgt | |
| | aggtgtgccaagtgtttctgttatcctacaaagcgaagaataaggaggaggccccgaaa | |
| | cctgaccatcttgagtctccccgaagatgtgctctttcacatcctgaaatggctttctgtaga | |
| | ggacatcctggccgtccgagctgtacactcccagctgaaggacctggtggacaaccacg | |
| | ccagtgtgtgggcatgtgccagcttccaggagctgtggccgtctccagggaacctgaag | |
| | ctctttgaaagggctgctgaaaaggggaatttcgaagctgctgtgaagctgggcatagc | |
| | ctacctctacaatgaaggcctgtctgtgtctgatgaggcccgcgcagaagtgaatggcct | |
| | gaaggcctctcgcttcttcagtctcgctgagcggctgaatgtgggtgccgcacctttcatct | |
| | ggctcttcatccgccctccgtggtcggtgagcggaagctgctgcaaggccgtggttcacg | |
| | agagcctcagggcagagtgccagctgcagaggactcacaaagcatccatattgcactgc | |
| | ttgggcagagtgctgagtctgttcgaggatgaggagaagcagcagcaggcccatgacc | |
| | tgtttgaggaggctgctcatcagggatgtctgaccagctcctacctcctctgggaaagcg | |
| | acaggaggacagatgtgtcagatcctgggcgatgcctccacagcttccgaaaactcagg | |
| | gactacgctgccaaaggctgctgggaagcgcagctgtctttagccaaagcctgtgcaaat | |
| | gcaaaccagcttggactggaggtgagagcttccagtgagatcgtctgccagctatttcag | |
| | gcttcccaggctgtcagtaaacaacaagtcttctccgtgcagaagggactcaatgacaca | |
| | atgaggtacattctgatcgactggctggtggaagttgccaccatgaaggacttcacaagc | |
| | ctgtgcctgcacctgaccgtggagtgtgtggaccggtacctgcggaggaggctggtgcc | |
| | gcggtacaggctccagctgctgggcatcgcctgcatggtcatctgcacccggtttatcagt | |
| | aaagagatcctgaccatccgggaggccgtatggctcacggacaacacttacaagtacga | |
| | ggacctggtgagaatgatgggcgagatcgtctccgccttggaagggaagattcgagtcc | |
| | ccactgtggtggattacaaggaggtcctgctgacgctagtccctgtggagctgagaaccc | |
| | agcacctgtgcagcttcctctgcgagctctccctgctgcacaccagcctgtccgcctacgcc | |
| | ccagcccgcctggctgccgcagccctgctcctggccagactgacgcacgggcagacaca | |
| | gccctggaccactcagctgtgggacctcaccggattctcctatgaagacctcattccctgc | |
| | gtcttgagcctccataagaagtgcttccatgatgacgcccccaaggactacaggcaagtc | |
| | tctctgaccgccgtgaagcagcggtttgaggacaagcgctatggagaaatcagccagg | |
| | aagaggtgctgagctacagccagttgtgtgctgcattaggagtgacacaagacagcccc | |
| | gaccccccgactttcctcagcacaggggagatccacgccttcctcagctctccctcggggc | |
| | ggagaaccaaacggaagcgggagaacagcctccaggaagacagaggcagcttcgtta | |
| | ccacccccactgcggagctgtccagccaggaggagacgctgctgggcagcttcctcgac | |
| | tggagcctggactgctgctctggctatgaaggcgaccaggagagtgagggcgagaagg | |
| | agggcgacgtgacagctcccagcggcatcctcgatgtcaccgtggtctacctgaaccca | |
| | gaacagcattgctgccaggaatccagtgatgaggaggcttgtccagaggacaagggac | |
| | cccaggacccacaggcactggcgctggacacccagatccctgcaacccctggacccaaa | |
| | cccctggtccgcaccagccgggagccagggaaggacgtcacgacctcagggtactcctc | |
| | cgtcagcaccgcaagtcccacaagctccgtggacggtggcttgggggccctgccccaac | |
| | ctacctcagtgctgtccctggacagtgactcgcacacacagccctgccaccatcaggcca | |
| | ggaagtcatgtttacagtgtcgtcccccaagtcccccggagagcagtgttccccagcaac | |
| | aggtgaagcggataaacctatgcatacacagtgaggaggaggacatgaacctgggcct | |
| | tgtgaggctgtaagtgtgtcagcacatttgccgcagtggatgtgtactgagggggctgga | |
| | ggcgaagggtgggagcatagcataggaacgctgcatagaccatggaggcctttgcgca | |
| | gagagcagagaggatgacttgcggccaccaagtttctgtctccgcgggagtcccgtgca | |
| | agccatcagaatgttgaaatgagggtgaagagctcagatccctctctttggaaagtttag | |
| | cctggaagcagttggccacactgtgtggagggcacctctctgtcccttccgtgtctcactgt | |
| | ctctggaagcttcagcccatgtgtgtcctggtgttcccagccccaccagagccccgtgccg | |
| | ggagctgacagctttcacgcttaaggcacgtgtgacctgggtagtcagacaccacttgag | |
| | cccctgcccacatctgctggtttggggcttcagtggggagctgacagctgtgagcacacc | |
| | actgtcccctcatccacctcggcctgcatggggcacccacttccttctgggtggggcttcca | |
| | tggtaagggggcctgcgtccctgcacactgcgaggactgccttggccacaggcccactcc | |
| | ctacgacacgtgactcgttttagagctctgtcccagaggcgttcgtatgtgacccacagat | |
| | ggcgtcaatgtgaacacctctctttgtgctgaatttctgggccattcttttcctgtcttatttct | |
| | aaatttccttcttccaagatgaaaacaaaagaaaaacttaaaacagaaggtattaaaaa | |
| | aacaagagattcccaccattatttaggttcacctgcaaaacaaaaatcttactccagcccc | |
| | tcaatgccatcctgacacactttatgcaaaaagaattttcccagataggctagccagaaa | |
| | aaacttcaagtcctctgtaacatctgaggtgaccaagaggcagaagagcagagcagtc | |
| | gggggccgtgtcctggctgatcccaactgcagctctgctgtgggggcccgtgggaggga | |
| | ggcagacccctgggctttcctgctggccacggagactctgctcctgcatggaaagggag | |
| | cctgggagccagcagcccacgcctggggagcctgcctggggccatgtgaccatggcctc | |
| | tccctgggaacgggctgaccacaacacaccctgctgccatccacttctgtttactctgcaa | |
| | atgtaagaaagaaccacttggccagaagtgtcccccagatgcttttttttttttttttttgga | |
| | gacagttttgctcttgtctccccggctggagtgcagtggcatgatctcaactctcaactcac | |
| | tgtaacctccgcctcccggatactcctgcctcagcctcctgggtagctgggattacaagca | |
| | cccaaccacgcccagctaatttttgtattttcggtagagacgggatttcaccatgttggcca | |
| | ggctagtctcgaactcatgacctcaagtgatccgcccacttcggtctcccaaagtgctggg | |
| | attacaggcatgagccacggcgcctggcccccaaatgctcttgaaccggaaacccaggg | |
| | atgggagatgctcactgagctgctgcttttatgtgtgctggtgctatgtgtgttcatgtccgc | |
| | ggcagctgtctttttgctactataagggaattctggccaccctgggtggggtgtggtcggg | |
| | gtgagaacccaagcgttggaactgtagacccgtcctgtcgactgtgtgcccctgggcatg | |
| | tgtgagcctcagtttcctcatctgtaaggggggcaatgatacctacctcacaggggtgttg | |
| | tgaggattaaatgtgaggaggatagtggcagatg | |
| NM_001323538 | atggggagcggcggcgtggtccactgtaggtgtgccaagtgtttctgttatcctacaaag | 2 |
| CDS | cgaagaataaggaggaggccccgaaacctgaccatcttgagtctccccgaagatgtgct | |
| | ctttcacatcctgaaatggctttctgtagaggacatcctggccgtccgagctgtacactccc | |
| | agctgaaggacctggtggacaaccacgccagtgtgtgggcatgtgccagcttccaggag | |
| | ctgtggccgtctccagggaacctgaagctctttgaaagggctgctgaaaaggggaatttc | |
| | gaagctgctgtgaagctgggcatagcctacctctacaatgaaggcctgtctgtgtctgatg | |
| | aggcccgcgcagaagtgaatggcctgaaggcctctcgcttcttcagtctcgctgagcggc | |
| | tgaatgtgggtgccgcacctttcatctggctcttcatccgccctccgtggtcggtgagcgga | |
| | agctgctgcaaggccgtggttcacgagagcctcagggcagagtgccagctgcagagga | |
| | ctcacaaagcatccatattgcactgcttgggcagagtgctgagtctgttcgaggatgagg | |
| | agaagcagcagcaggcccatgacctgtttgaggaggctgctcatcagggatgtctgacc | |
| | agctcctacctcctctgggaaagcgacaggaggacagatgtgtcagatcctgggcgatg | |
| | cctccacagcttccgaaaactcagggactacgctgccaaaggctgctgggaagcgcagc | |
| | tgtctttagccaaagcctgtgcaaatgcaaaccagcttggactggaggtgagagcttcca | |
| | gtgagatcgtctgccagctatttcaggcttcccaggctgtcagtaaacaacaagtcttctcc | |
| | gtgcagaagggactcaatgacacaatgaggtacattctgatcgactggctggtggaagt | |
| | tgccaccatgaaggacttcacaagcctgtgcctgcacctgaccgtggagtgtgtggaccg | |
| | gtacctgcggaggaggctggtgccgcggtacaggctccagctgctgggcatcgcctgca | |
| | tggtcatctgcacccggtttatcagtaaagagatcctgaccatccgggaggccgtatggct | |
| | cacggacaacacttacaagtacgaggacctggtgagaatgatgggcgagatcgtctccg | |
| | ccttggaagggaagattcgagtccccactgtggtggattacaaggaggtcctgctgacg | |
| | ctagtccctgtggagctgagaacccagcacctgtgcagcttcctctgcgagctctccctgct | |
| | gcacaccagcctgtccgcctacgccccagcccgcctggctgccgcagccctgctcctggc | |
| | cagactgacgcacgggcagacacagccctggaccactcagctgtgggacctcaccgga | |
| | ttctcctatgaagacctcattccctgcgtcttgagcctccataagaagtgcttccatgatgac | |
| | gcccccaaggactacaggcaagtctctctgaccgccgtgaagcagcggtttgaggacaa | |
| | gcgctatggagaaatcagccaggaagaggtgctgagctacagccagttgtgtgctgcat | |
| | taggagtgacacaagacagccccgaccccccgactttcctcagcacaggggagatccac | |
| | gccttcctcagctctccctcggggcggagaaccaaacggaagcgggagaacagcctcc | |
| | aggaagacagaggcagcttcgttaccacccccactgcggagctgtccagccaggagga | |
| | gacgctgctgggcagcttcctcgactggagcctggactgctgctctggctatgaaggcga | |
| | ccaggagagtgagggcgagaaggagggcgacgtgacagctcccagcggcatcctcga | |
| | tgtcaccgtggtctacctgaacccagaacagcattgctgccaggaatccagtgatgagga | |
| | ggcttgtccagaggacaagggaccccaggacccacaggcactggcgctggacacccag | |
| | atccctgcaacccctggacccaaacccctggtccgcaccagccgggagccagggaagg | |
| | acgtcacgacctcagggtactcctccgtcagcaccgcaagtcccacaagctccgtggacg | |
| | gtggcttgggggccctgccccaacctacctcagtgctgtccctggacagtgactcgcaca | |
| | cacagccctgccaccatcaggccaggaagtcatgtttacagtgtcgtcccccaagtccccc | |
| | ggagagcagtgttccccagcaacaggtgaagcggataaacctatgcatacacagtgag | |
| | gaggaggacatgaacctgggccttgtgaggctgtaa | |
| NP_001752 | MGSGGVVHCRCAKCFCYPTKRRIRRRPRNLTILSLPEDVLFHILKWLSV | 3 |
| | EDILAVRAVHSQLKDLVDNHASVWACASFQELWPSPGNLKLFERAAE | |
| | KGNFEAAVKLGIAYLYNEGLSVSDEARAEVNGLKASRFFSLAERLNVGA | |
| | APFIWLFIRPPWSVSGSCCKAVVHESLRAECQLQRTHKASILHCLGRVL | |
| | SLFEDEEKQQQAHDLFEEAAHQGCLTSSYLLWESDRRTDVSDPGRCL | |
| | HSFRKLRDYAAKGCWEAQLSLAKACANANQLGLEVRASSEIVCQLFQA | |
| | SQAVSKQQVFSVQKGLNDTMRYILIDWLVEVATMKDFTSLCLHLTVEC | |
| | VDRYLRRRLVPRYRLQLLGIACMVICTRFISKEILTIREAVWLTDNTYKY | |
| | EDLVRMMGEIVSALEGKIRVPTVVDYKEVLLTLVPVELRTQHLCSFLCEL | |
| | SLLHTSLSAYAPARLAAAALLLARLTHGQTQPWTTQLWDLTGFSYEDLI | |
| | PCVLSLHKKCFHDDAPKDYRQVSLTAVKQRFEDKRYGEISQEEVLSYS | |
| | QLCAALGVTQDSPDPPTFLSTGEIHAFLSSPSGRRTKRKRENSLQEDR | |
| | GSFVTTPTAELSSQEETLLGSFLDWSLDCCSGYEGDQESEGEKEGDVT | |
| | APSGILDVTVVYLNPEQHCCQESSDEEACPEDKGPQDPQALALDTQIP | |
| | ATPGPKPLVRTSREPGKDVTTSGYSSVSTASPTSSVDGGLGALPQPTS | |
| | VLSLDSDSHTQPCHHQARKSCLQCRPPSPPESSVPQQQVKRINLCIHS | |
| | EEEDMNLGLVRL | |
| NM_001323538 | ggtctgcgcctgcgcgagggctacgcgcgctccggccggggcgcgggcgcgctctcag | 4 |
| | gcgggctccggcggcagcgacgcgagcgcggcgatggggagcggcggcgtggtccac | |
| | tgtaggtgtgccaagtgtttctgttatcctacaaagcgaagaataaggaggaggccccg | |
| | aaacctgaccatcttgagtctccccgaagatgtgctctttcacatcctgaaatggctttctgt | |
| | agaggacatcctggccgtccgagctggctgctgaaaaggggaatttcgaagctgctgtg | |
| | aagctgggcatagcctacctctacaatgaaggcctgtctgtgtctgatgaggcccgcgca | |
| | gaagtgaatggcctgaaggcctctcgcttcttcagtctcgctgagcggctgaatgtgggt | |
| | gccgcacctttcatctggctcttcatccgccctccgtggtcggtgagcggaagctgctgca | |
| | aggccgtggttcacgagagcctcagggcagagtgccagctgcagaggactcacaaagc | |
| | atccatattgcactgcttgggcagagtgctgagtctgttcgaggatgaggagaagcagc | |
| | agcaggcccatgacctgtttgaggaggctgctcatcagggatgtctgaccagctcctacc | |
| | tcctctgggaaagcgacaggaggacagatgtgtcagatcctgggcgatgcctccacagc | |
| | ttccgaaaactcagggactacgctgccaaaggctgctgggaagcgcagctgtctttagcc | |
| | aaagcctgtgcaaatgcaaaccagcttggactggaggtgagagcttccagtgagatcgt | |
| | ctgccagctatttcaggcttcccaggctgtcagtaaacaacaagtcttctccgtgcagaag | |
| | ggactcaatgacacaatgaggtacattctgatcgactggctggtggaagttgccaccatg | |
| | aaggacttcacaagcctgtgcctgcacctgaccgtggagtgtgtggaccggtacctgcgg | |
| | aggaggctggtgccgcggtacaggctccagctgctgggcatcgcctgcatggtcatctgc | |
| | acccggtttatcagtaaagagatcctgaccatccgggaggccgtatggctcacggacaa | |
| | cacttacaagtacgaggacctggtgagaatgatgggcgagatcgtctccgccttggaag | |
| | ggaagattcgagtccccactgtggtggattacaaggaggtcctgctgacgctagtccctg | |
| | tggagctgagaacccagcacctgtgcagcttcctctgcgagctctccctgctgcacaccag | |
| | cctgtccgcctacgccccagcccgcctggctgccgcagccctgctcctggccagactgac | |
| | gcacgggcagacacagccctggaccactcagctgtgggacctcaccggattctcctatga | |
| | agacctcattccctgcgtcttgagcctccataagaagtgcttccatgatgacgcccccaag | |
| | gactacaggcaagtctctctgaccgccgtgaagcagcggtttgaggacaagcgctatgg | |
| | agaaatcagccaggaagaggtgctgagctacagccagttgtgtgctgcattaggagtga | |
| | cacaagacagccccgaccccccgactttcctcagcacaggggagatccacgccttcctca | |
| | gctctccctcggggcggagaaccaaacggaagcgggagaacagcctccaggaagaca | |
| | gaggcagcttcgttaccacccccactgcggagctgtccagccaggaggagacgctgctg | |
| | ggcagcttcctcgactggagcctggactgctgctctggctatgaaggcgaccaggagag | |
| | tgagggcgagaaggagggcgacgtgacagctcccagcggcatcctcgatgtcaccgtg | |
| | gtctacctgaacccagaacagcattgctgccaggaatccagtgatgaggaggcttgtcca | |
| | gaggacaagggaccccaggacccacaggcactggcgctggacacccagatccctgca | |
| | acccctggacccaaacccctggtccgcaccagccgggagccagggaaggacgtcacga | |
| | cctcagggtactcctccgtcagcaccgcaagtcccacaagctccgtggacggtggcttgg | |
| | gggccctgccccaacctacctcagtgctgtccctggacagtgactcgcacacacagccct | |
| | gccaccatcaggccaggaagtcatgtttacagtgtcgtcccccaagtcccccggagagca | |
| | gtgttccccagcaacaggtgaagcggataaacctatgcatacacagtgaggaggagga | |
| | catgaacctgggccttgtgaggctgtaagtgtgtcagcacatttgccgcagtggatgtgta | |
| | ctgagggggctggaggcgaagggtgggagcatagcataggaacgctgcatagaccat | |
| | ggaggcctttgcgcagagagcagagaggatgacttgcggccaccaagtttctgtctccg | |
| | cgggagtcccgtgcaagccatcagaatgttgaaatgagggtgaagagctcagatccctc | |
| | tctttggaaagtttagcctggaagcagttggccacactgtgtggagggcacctctctgtcc | |
| | cttccgtgtctcactgtctctggaagcttcagcccatgtgtgtcctggtgttcccagccccac | |
| | cagagccccgtgccgggagctgacagctttcacgcttaaggcacgtgtgacctgggtagt | |
| | cagacaccacttgagcccctgcccacatctgctggtttggggcttcagtggggagctgac | |
| | agctgtgagcacaccactgtcccctcatccacctcggcctgcatggggcacccacttcctt | |
| | ctgggtggggcttccatggtaagggggcctgcgtccctgcacactgcgaggactgccttg | |
| | gccacaggcccactccctacgacacgtgactcgttttagagctctgtcccagaggcgttcg | |
| | tatgtgacccacagatggcgtcaatgtgaacacctctctttgtgctgaatttctgggccatt | |
| | cttttcctgtcttatttctaaatttccttcttccaagatgaaaacaaaagaaaaacttaaaac | |
| | agaaggtattaaaaaaacaagagattcccaccattatttaggttcacctgcaaaacaaaa | |
| | atcttactccagcccctcaatgccatcctgacacactttatgcaaaaagaattttcccagat | |
| | aggctagccagaaaaaacttcaagtcctctgtaacatctgaggtgaccaagaggcagaa | |
| | gagcagagcagtcgggggccgtgtcctggctgatcccaactgcagctctgctgtggggg | |
| | cccgtgggagggaggcagacccctgggctttcctgctggccacggagactctgctcctgc | |
| | atggaaagggagcctgggagccagcagcccacgcctggggagcctgcctggggccat | |
| | gtgaccatggcctctccctgggaacgggctgaccacaacacaccctgctgccatccacttc | |
| | tgtttactctgcaaatgtaagaaagaaccacttggccagaagtgtcccccagatgctttttt | |
| | ttttttttttttggagacagttttgctcttgtctccccggctggagtgcagtggcatgatctca | |
| | actctcaactcactgtaacctccgcctcccggatactcctgcctcagcctcctgggtagctg | |
| | ggattacaagcacccaaccacgcccagctaatttttgtattttcggtagagacgggatttc | |
| | accatgttggccaggctagtctcgaactcatgacctcaagtgatccgcccacttcggtctc | |
| | ccaaagtgctgggattacaggcatgagccacggcgcctggcccccaaatgctcttgaac | |
| | cggaaacccagggatgggagatgctcactgagctgctgcttttatgtgtgctggtgctatg | |
| | tgtgttcatgtccgcggcagctgtctttttgctactataagggaattctggccaccctgggt | |
| | ggggtgtggtcggggtgagaacccaagcgttggaactgtagacccgtcctgtcgactgt | |
| | gtgcccctgggcatgtgtgagcctcagtttcctcatctgtaaggggggcaatgatacctac | |
| | ctcacaggggtgttgtgaggattaaatgtgaggaggatagtggcaaaaaaaaaaaaaa | |
| | aaaa | |
| NM_001323538 | atgaggtacattctgatcgactggctggtggaagttgccaccatgaaggacttcacaagc | 5 |
| CDS | ctgtgcctgcacctgaccgtggagtgtgtggaccggtacctgcggaggaggctggtgcc | |
| | gcggtacaggctccagctgctgggcatcgcctgcatggtcatctgcacccggtttatcagt | |
| | aaagagatcctgaccatccgggaggccgtatggctcacggacaacacttacaagtacga | |
| | ggacctggtgagaatgatgggcgagatcgtctccgccttggaagggaagattcgagtcc | |
| | ccactgtggtggattacaaggaggtcctgctgacgctagtccctgtggagctgagaaccc | |
| | agcacctgtgcagcttcctctgcgagctctccctgctgcacaccagcctgtccgcctacgcc | |
| | ccagcccgcctggctgccgcagccctgctcctggccagactgacgcacgggcagacaca | |
| | gccctggaccactcagctgtgggacctcaccggattctcctatgaagacctcattccctgc | |
| | gtcttgagcctccataagaagtgcttccatgatgacgcccccaaggactacaggcaagtc | |
| | tctctgaccgccgtgaagcagcggtttgaggacaagcgctatggagaaatcagccagg | |
| | aagaggtgctgagctacagccagttgtgtgctgcattaggagtgacacaagacagcccc | |
| | gaccccccgactttcctcagcacaggggagatccacgccttcctcagctctccctcggggc | |
| | ggagaaccaaacggaagcgggagaacagcctccaggaagacagaggcagcttcgtta | |
| | ccacccccactgcggagctgtccagccaggaggagacgctgctgggcagcttcctcgac | |
| | tggagcctggactgctgctctggctatgaaggcgaccaggagagtgagggcgagaagg | |
| | agggcgacgtgacagctcccagcggcatcctcgatgtcaccgtggtctacctgaaccca | |
| | gaacagcattgctgccaggaatccagtgatgaggaggcttgtccagaggacaagggac | |
| | cccaggacccacaggcactggcgctggacacccagatccctgcaacccctggacccaaa | |
| | cccctggtccgcaccagccgggagccagggaaggacgtcacgacctcagggtactcctc | |
| | cgtcagcaccgcaagtcccacaagctccgtggacggtggcttgggggccctgccccaac | |
| | ctacctcagtgctgtccctggacagtgactcgcacacacagccctgccaccatcaggcca | |
| | ggaagtcatgtttacagtgtcgtcccccaagtcccccggagagcagtgttccccagcaac | |
| | aggtgaagcggataaacctatgcatacacagtgaggaggaggacatgaacctgggcct | |
| | tgtgaggctgtaa | |
| NP_001310467 | MRYILIDWLVEVATMKDFTSLCLHLTVECVDRYLRRRLVPRYRLQLLGI | 6 |
| | ACMVICTRFISKEILTIREAVWLTDNTYKYEDLVRMMGEIVSALEGKIRV | |
| | PTVVDYKEVLLTLVPVELRTQHLCSFLCELSLLHTSLSAYAPARLAAAAL | |
| | LLARLTHGQTQPWTTQLWDLTGFSYEDLIPCVLSLHKKCFHDDAPKDY | |
| | RQVSLTAVKQRFEDKRYGEISQEEVLSYSQLCAALGVTQDSPDPPTFLS | |
| | TGEIHAFLSSPSGRRTKRKRENSLQEDRGSFVTTPTAELSSQEETLLGS | |
| | FLDWSLDCCSGYEGDQESEGEKEGDVTAPSGILDVTVVYLNPEQHCC | |
| | QESSDEEACPEDKGPQDPQALALDTQIPATPGPKPLVRTSREPGKDVT | |
| | TSGYSSVSTASPTSSVDGGLGALPQPTSVLSLDSDSHTQPCHHQARK | |
| | SCLQCRPPSPPESSVPQQQVKRINLCIHSEEEDMNLGLVRL | |
| | MGSGGVVHCRCAKCFCYPTKRRIRRRPRNLTILSLPEDVLFHILKWLSV | 7 |
| | EDILAVRAVHSQLKDLVDNHASVWACASFQELWPSPGNLKLFERAAE | |
| | KGNFEAAVKLGIAYLYNEGLSVSDEARAEVNGLKASRFFSLAERLNVGA | |
| | APFIWLFIRPPWSVSGSCCKAVVHESLRAECQLQRTHKASILHCLGRVL | |
| | SLFEDEEKQQQAHDLFEEAAHQGCLTSSYLLWESDRRTDVSDPGRCL | |
| | HSFRKLRDYAAKGCWEAQLSLAKACANANQLGLEVRASSEIVCQLFQA | |
| | SQAVSKQQVFSVQKGLNDTAAYILIDWLVEVATMKDFTSLCLHLTVEC | |
| | VDRYLRRRLVPRYRLQLLGIACMVICTRFISKEILTIREAVWLTDNTYKY | |
| | EDLVRMMGEIVSALEGKIRVPTVVDYKEVLLTLVPVELRTQHLCSFLCEL | |
| | SLLHTSLSAYAPARLAAAALLLARLTHGQTQPWTTQLWDLTGFSYEDLI | |
| | PCVLSLHKKCFHDDAPKDYRQVSLTAVKQRFEDKRYGEISQEEVLSYS | |
| | QLCAALGVTQDSPDPPTFLSTGEIHAFLSSPSGRRTKRKRENSLQEDR | |
| | GSFVTTPTAELSSQEETLLGSFLDWSLDCCSGYEGDQESEGEKEGDVT | |
| | APSGILDVTWYLNPEQHCCQESSDEEACPEDKGPQDPQALALDTQIP | |
| | ATPGPKPLVRTSREPGKDVTTSGYSSVSTASPTSSVDGGLGALPQPTS | |
| | VLSLDSDSHTQPCHHQARKSCLQCRPPSPPESSVPQQQVKRINLCIHS | |
| | EEEDMNLGLVRL | |

### DETAILED DESCRIPTION

### I. Definitions

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, preferred methods and materials are described. For the purposes of the present disclosure, the following terms are defined below.

The articles “a”, “an” and “the” refer to one or to more than one (i.e., to at least one) of the grammatical object of the article, unless context clearly indicates otherwise.

As used herein, “and/or” refers to and encompasses any and all possible combinations of one or more of the associated listed items, as well as the lack of combinations when interpreted in the alternative (or).

Further, the terms “about” and “approximate”, as used herein when referring to a measurable value such as an amount, dose, time, temperature, activity, level, number, frequency, percentage, dimension, size, amount, weight, position, length and the like, is meant to encompass variations of ±15%, ±10%, ±5%, ±1%, ±0.5%, or even ±0.1% of the specified amount, dose, time, temperature, activity, level, number, frequency, percentage, dimension, size, amount, weight, position, length and the like.

The term “activity” as used herein shall be understood as a measure for the ability of a transcription product or a translation product to produce a biological effect or a measure for a level of biologically active molecules. Accordingly, in the context of cyclin F, the term “activity” refers to any one or more of the following activities: (1) associating with other subunits to form a Skp1-Cul1-F-box (SCF) E3 ubiquitin-protein ligase complex (SCF^{Cyclin F}); (2) suppressing B-Myb activity to promote cell cycle checkpoint control; (3) interacting with a substrate (e.g., CDC6, RRM2, CP110, and SLBP, as well as TDP-43) to promote ubiquitylation and degradation of the substrate; and (4) directly binding to TDP-43, as disclosed herein.

As used herein, the term “administered” refers to the placement of an agent described herein, into a subject by a method or route which results in at least partial localization of the compound at a desired site. An agent described herein can be administered by any appropriate route which results in effective treatment in the subject, i.e., administration results in delivery to a desired location in the subject where at least a portion of the composition delivered. Exemplary modes of administration include, but are not limited to, injection, infusion, instillation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebrospinal, and intrasternal injection and infusion.

The terms “administration concurrently” or “administering concurrently” or “co-administering” and the like refer to the administration of a single composition containing two or more actives, or the administration of each active as separate compositions and/or delivered by separate routes either contemporaneously or simultaneously or sequentially within a short enough period of time that the effective result is equivalent to that obtained when all such actives are administered as a single composition. By “simultaneously” is meant that the active agents are administered at substantially the same time, and desirably together in the same formulation. By “contemporaneously” it is meant that the active agents are administered closely in time, e.g., one agent is administered within from about one minute to within about one day before or after another. Any contemporaneous time is useful. However, it will often be the case that when not administered simultaneously, the agents will be administered within about one minute to within about eight hours and suitably within less than about one to about four hours. When administered contemporaneously, the agents are suitably administered at the same site on the subject. The term “same site” includes the exact location, but can be within about 0.5 to about 15 centimeters, preferably from within about 0.5 to about 5 centimeters. The term “separately” as used herein means that the agents are administered at an interval, for example at an interval of about a day to several weeks or months. The active agents may be administered in either order. The term “sequentially” as used herein means that the agents are administered in sequence, for example at an interval or intervals of minutes, hours, days or weeks. If appropriate the active agents may be administered in a regular repeating cycle.

The term “agent” includes a compound that induces a desired pharmacological and/or physiological effect. The term also encompass pharmaceutically acceptable and pharmacologically active ingredients of those compounds specifically mentioned herein including but not limited to salts, esters, amides, prodrugs, active metabolites, analogs and the like. When the above term is used, then it is to be understood that this includes the active agent per se as well as pharmaceutically acceptable, pharmacologically active salts, esters, amides, prodrugs, metabolites, analogs, etc. The term “agent” is not to be construed narrowly but extends to small molecules, proteinaceous molecules such as peptides, polypeptides and proteins as well as compositions comprising them and genetic molecules such as RNA, DNA and mimetics and chemical analogs thereof as well as cellular agents. The term “agent” includes a cell that is capable of producing and secreting a polypeptide referred to herein as well as a polynucleotide comprising a nucleotide sequence that encodes that polypeptide. Thus, the term “agent” extends to nucleic acid constructs including vectors such as viral or non-viral vectors, expression vectors and plasmids for expression in and secretion in a range of cells. As used herein, the terms “candidate agent” and “test agent” are used interchangeably to refer to agents and/or compositions that are to be screened for their ability to stimulate and/or increase and/or promote motor neuron survival, and/or to inhibit or reduce motor neuron degeneration, and/or to inhibit or reduce abnormal protein accumulation in motor neurons.

As used herein, an “agent that enhances the level or activity of cyclin F” or “cyclin F-enhancing agent” refers to an agent that increases the level of cyclin F mRNA or protein, an activity of cyclin F, the half-life of cyclin F mRNA or protein, or the binding of cyclin F to another molecule (e.g., a substrate for cyclin F such as TDP-43 and/or other components of the SCF^{Cyclin F} complex). For example, the agent may directly or indirectly enhance the ability of cyclin F to associate with other components of the SCF^{Cyclin F} complex and ubiquitinate proteins for clearance by the proteasome. Expression levels of mRNA can be determined using standard RNase protection assays or in situ hybridization assays, and the level of protein can be determined using standard Western or immunohistochemistry analysis. The ubiquitination level of a protein can also be measured using standard assays. In some embodiments, an agent that enhances the level or activity of cyclin F increases cyclin F activity by at least 20, 40, 60, 80, or 90%. In some embodiments, the level of cyclin F is at least 2, 3, 5, 10, 20, or 50-fold higher in the presence of the cyclin F-enhancing agent.

The terms “cis-acting element”, “cis-acting sequence” or “cis-regulatory region” are used interchangeably herein to mean any sequence of nucleotides, which modulates transcriptional activity of an operably linked promoter and/or expression of an operably linked nucleotide sequence. Those skilled in the art will be aware that a ds-sequence may be capable of activating, silencing, enhancing, repressing or otherwise altering the level of expression and/or cell-type-specificity and/or developmental specificity of any nucleotide sequence, including coding and non-coding sequences.

By “coding sequence” is meant any nucleic acid sequence that contributes to the code for the polypeptide product of a gene or for the final mRNA product of a gene (e.g. the mRNA product of a gene following splicing). By contrast, the term “non-coding sequence” refers to any nucleic acid sequence that does not contribute to the code for the polypeptide product of a gene or for the final mRNA product of a gene.

Throughout this specification, unless the context requires otherwise, the words “comprise,” “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, the term “condition” includes anatomic and physiological deviations from the normal that constitute an impairment of the normal state of the living animal or one of its parts, that interrupts or modifies the performance of the bodily functions.

The terms “conditional expression”, “conditionally expressed” “conditionally expressing” and the like refer to the ability to activate or suppress expression of a gene of interest by the presence or absence of a stimulus or other signal (e.g., chemical, light, hormone, stress, or a pathogen). In specific embodiments, conditional expression of a nucleic acid sequence of interest is dependent on the presence of an inducer or the absence of an inhibitor.

A “conservative amino acid substitution” is one in which the amino acid residue is replaced with an amino acid residue having a similar side chain. Families of amino acid residues having similar side chains have been defined in the art, which can be generally sub-classified as follows:

TABLE 1
AMINO ACID SUB-CLASSIFICATION

| Sub-classes | Amino acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, Histidine |
| Small | Glycine, Serine, Alanine, Threonine, Proline |
| Polar/neutral | Asparagine, Histidine, Glutamine, Cysteine, Serine, |
| | Threonine |
| Polar/large | Asparagine, Glutamine |
| Hydrophobic | Tyrosine, Valine, Isoleucine, Leucine, Methionine, |
| | Phenylalanine, Tryptophan |
| Aromatic | Tryptophan, Tyrosine, Phenylalanine |
| Residues that | Glycine and Proline |
| influence chain | |
| orientation | |

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a functional polypeptide can readily be determined by assaying its activity. Conservative substitutions are shown in Table 2 under the heading of exemplary and preferred substitutions. Amino acid substitutions falling within the scope of the present disclosure, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants are screened for biological activity.

TABLE 2
EXEMPLARY AND PREFERRED
AMINO ACID SUBSTITUTIONS

| Original | | |
|---|---|---|
| Residue | Exemplary Substitutions | Preferred Substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, Norleu | Leu |
| Leu | Norleu, Ile, Val, Met, Ala, Phe | Ile |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleu | Leu |

The term “contacting” or “contact” as used herein in connection with contacting a motor neuron or motor neuron surrogate cell includes subjecting the motor neuron or surrogate cell to an appropriate culture media which comprises the indicated compound and/or agent. Where the motor neuron or surrogate cell is in vivo, “contacting” or “contact” includes administering the compound and/or agent in a pharmaceutical composition to a subject via an appropriate administration route such that the compound and/or agent contacts the motor neuron or surrogate cell in vivo. In specific embodiments, the contacted motor neuron or surrogate cell are assayed for cell survival. Measurement of cell survival can be based on the number of viable cells after period of time has elapsed after contacting of cells with a compound or agent. For example, number of viable cells can be counted after about at least 5 minutes, 10 minutes, 20 minutes, 30 minutes, 40 minutes, 50 minutes, 1 hour, 2 hours, 4 hours, 6 hours, 12 hours, 24 hours, 2 days, 3 days or more and compared to number of viable cells in a non-treated control.

The term “construct” refers to a recombinant genetic molecule including one or more isolated nucleic acid sequences from different sources. Thus, constructs are chimeric molecules in which two or more nucleic acid sequences of different origin are assembled into a single nucleic acid molecule and include any construct that contains (1) nucleic acid sequences, including regulatory and coding sequences that are not found together in nature (i.e., at least one of the nucleotide sequences is heterologous with respect to at least one of its other nucleotide sequences), or (2) sequences encoding parts of functional RNA molecules or proteins not naturally adjoined, or (3) parts of promoters that are not naturally adjoined. Representative constructs include any recombinant nucleic acid molecule such as a plasmid, cosmid, virus, autonomously replicating polynucleotide molecule, phage, or linear or circular single stranded or double stranded DNA or RNA nucleic acid molecule, derived from any source, capable of genomic integration or autonomous replication, comprising a nucleic acid molecule where one or more nucleic acid molecules have been operably linked. Constructs of the present disclosure will generally include the necessary elements to direct expression of a nucleic acid sequence of interest that is also contained in the construct, such as, for example, a target nucleic acid sequence or a modulator nucleic acid sequence. Such elements may include control elements such as a promoter that is operably linked to (so as to direct transcription of) the nucleic acid sequence of interest, and often includes a polyadenylation sequence as well. Within certain embodiments of the disclosure, the construct may be contained within a vector. In addition to the components of the construct, the vector may include, for example, one or more selectable markers, one or more origins of replication, such as prokaryotic and eukaryotic origins, at least one multiple cloning site, and/or elements to facilitate stable integration of the construct into the genome of a host cell. Two or more constructs can be contained within a single nucleic acid molecule, such as a single vector, or can be containing within two or more separate nucleic acid molecules, such as two or more separate vectors. An “expression construct” generally includes at least a control sequence operably linked to a nucleotide sequence of interest. In this manner, for example, promoters in operable connection with the nucleotide sequences to be expressed are provided in expression constructs for expression in an organism or part thereof including a host cell. For the practice of the methods of the present disclosure, conventional compositions and methods for preparing and using constructs and host cells are well known to one skilled in the art, see for example, Molecular Cloning: A Laboratory Manual, 3rd edition Volumes 1, 2, and 3. J. F. Sambrook, D. W. Russell, and N. Irwin, Cold Spring Harbor Laboratory Press, 2000.

The term “control neuron” as used herein means a neuron (e.g., a motor neuron) from one or more healthy subjects or subjects not having a neurodegenerative condition and/or not having a TDP-43 proteinopathy (e.g., control subjects).

By “corresponds to” or “corresponding to” is meant an amino acid sequence that displays substantial sequence similarity or identity to a reference amino acid sequence. In general the amino acid sequence will display at least about 70, 71, 72, 73, 74, 75, 76, 77, 78, 79, 80, 81, 82, 83, 84, 85, 86, 97, 88, 89, 90, 91, 92, 93, 94, 95, 96, 97, 98, 99% or even up to 100% sequence similarity or identity to at least a portion of the reference amino acid sequence.

The terms “decrease”, “reduce” or “inhibit” and their grammatical equivalents are all used herein generally to mean a decrease by a statistically significant amount. However, for avoidance of doubt, the terms “decrease”, “reduce” or “inhibit” and their grammatical equivalents mean a decrease by at least 10% as compared to a reference level, for example a decrease by at least about 20%, or at least about 30%, or at least about 40%, or at least about 50%, or at least about 60%, or at least about 70%, or at least about 80%, or at least about 90%, where the decrease is less than 100%. In one embodiment, the decrease includes a 100% decrease (e.g. absent level as compared to a reference sample), or any decrease between 10-100% as compared to a reference level.

As used herein, “dosage unit” refers to physically discrete units suited as unitary dosages for the subject to be treated; each unit containing a predetermined quantity of agent calculated to produce the desired therapeutic effect in association with the required pharmaceutical carrier.

As used herein, the term “effective amount” means an amount of the compound and/or agent which is effective to promote the survival of motor neuron cells or to inhibit or slow the death of such cells. Determination of an effective amount is well within the capability of those skilled in the art. Generally, an effective amount can vary with the subject's history, age, condition, sex, as well as the severity and type of the medical condition in the subject, and administration of other agents that inhibit pathological processes in neurodegenerative conditions.

As used herein, the terms “encode”, “encoding” and the like refer to the capacity of a nucleic acid to provide for another nucleic acid or a polypeptide. For example, a nucleic acid sequence is said to “encode” a polypeptide if it can be transcribed and/or translated to produce the polypeptide or if it can be processed into a form that can be transcribed and/or translated to produce the polypeptide. Such a nucleic acid sequence may include a coding sequence or both a coding sequence and a non-coding sequence. Thus, the terms “encode”, “encoding” and the like include a RNA product resulting from transcription of a DNA molecule, a protein resulting from translation of a RNA molecule, a protein resulting from transcription of a DNA molecule to form a RNA product and the subsequent translation of the RNA product, or a protein resulting from transcription of a DNA molecule to provide a RNA product, processing of the RNA product to provide a processed RNA product (e.g., mRNA) and the subsequent translation of the processed RNA product.

As used herein, the phrase “enhancing motor neuron survival” refers to an increase in survival of motor neuron cells as compared to a control. In some embodiments, contacting of a motor neuron with a cyclin F-enhancing agent described herein results in at least about 10%, 20%, 30%, 40%, 50% 60%, 70%, 80%, 90%, 95%, 100%, 2-fold, 3-fold, 4-fold, 5-fold or more increase in motor neuron survival relative to non-treated control. Motor neuron survival can be assessed by for example (i) increased survival time of motor neurons in culture; (ii) increased production of a neuron-associated molecule in culture or in vivo, e.g., choline acetyltransferase, acetylcholinesterase and cyclin F; (iii) reduced abnormal accumulation of proteins including TDP-43 in culture or in vivo; or (iv) decreased symptoms of motor neuron dysfunction in vivo. Such effects may be measured by any method known in the art. In one non-limiting example, increased survival of motor neurons may be measured by the method described by Arakawa et al. (1990, J. Neurosci. 10:3507-3515); increased production of neuron-associated molecules may be measured by bioassay, enzymatic assay, antibody binding, Northern blot assay, etc., depending on the molecule to be measured; reduced abnormal accumulation of proteins may be assayed through detection of aggregated proteins in aggresomes and inclusion bodies as described for example by Shen et al. (2011, Cell Biochem Biophys 60:173-185), and motor neuron dysfunction may be measured by assessing the physical manifestation of motor neuron disorder. In one embodiment, the increase in motor neuron survival can be assessed by measuring the increase in cyclin F levels. Cell survival can also be measured by uptake of calcein AM, an analog of the viable dye, fluorescein diacetate. Calcein is taken up by viable cells and cleaved intracellularly to fluorescent salts which are retained by intact membranes of viable cells. Microscopic counts of viable neurons correlate directly with relative fluorescence values obtained with the fluorometric viability assay. This method thus provides a reliable and quantitative measurement of cell survival in the total cell population of a given culture (Bozyczko-Coyne et al., J. Neur. Meth. 50:205-216, 1993). Other methods of assessing cell survival are described in U.S. Pat. Nos. 5,972,639; 6,077,684 and 6,417,160, contents of which are incorporated herein by reference. In vivo motor neuron survival can be assessed by an increase in motor neuron, neuromotor or neuromuscular function in a subject. In one non-limiting example, motor neuron survival in a subject can be assessed by reversion, alleviation, amelioration, inhibition, slowing down or stopping of the progression, aggravation or severity of a condition associated with motor neuron dysfunction or death in a subject, e.g., ALS or FTD.

The term “endogenous” refers to a molecule (e.g., a nucleic acid, carbohydrate, lipid or polypeptide) that is present and/or naturally expressed within a host organism or cell thereof. For example, an “endogenous cyclin F” refers to a cyclin F polypeptide that is naturally expressed in a cell (e.g., a motor neuron).

As used herein, the term “exogenous” refers to a molecule (e.g., a nucleic acid, carbohydrate, lipid or polypeptide) that is introduced into a host cell. In specific embodiments, an exogenous polypeptide refers to a polypeptide that is expressed from a polynucleotide which is foreign to the cell into which it has been introduced, or a polynucleotide that is homologous to a sequence in the cell into which it is introduced but in a position within the host cell nucleic acid in which the polynucleotide is not normally found.

The term “expression” with respect to a gene sequence refers to transcription of the gene to produce a RNA transcript (e.g., mRNA, antisense RNA, siRNA, shRNA, miRNA, etc.) and, as appropriate, translation of a resulting mRNA transcript to a protein. Thus, as will be clear from the context, expression of a coding sequence results from transcription and translation of the coding sequence. Conversely, expression of a non-coding sequence results from the transcription of the non-coding sequence.

As used herein, the term “gene” refers to a nucleic acid molecule capable of being used to produce mRNA, antisense RNA, siRNA, shRNA, miRNA, and the like, and in some embodiments, polypeptide. Genes may or may not be capable of being used to produce a functional protein. Genes can include both coding and non-coding regions (e.g., introns, regulatory elements including promoters, enhancers, termination sequences and 5′ and 3′ untranslated regions). In certain embodiments, the term “gene” includes within its scope the open reading frame encoding specific polypeptides, introns, and adjacent 5′ and 3′ non-coding nucleotide sequences involved in the regulation of expression. In this regard, the gene may further comprise control sequences such as promoters, enhancers, termination and/or polyadenylation signals that are naturally associated with a given gene, or heterologous control sequences. The gene sequences may be cDNA or genomic DNA or a fragment thereof. The gene may be introduced into an appropriate vector for extrachromosomal maintenance or for introduction into a host.

The terms “increase”, “enhance”, or “activate” and their grammatical equivalents are all used herein to generally mean an increase by a statically significant amount; for the avoidance of any doubt, the terms “increase”, “enhance”, or “activate” and their grammatical equivalents mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the phrase “inhibiting motor neuron degeneration” refers to reducing loss of motor neuron viability, reducing loss of motor neuron function and/or reducing loss of the number of motor neurons. In some embodiments, contacting of a motor neuron with an agent described herein results in at least about 10%, 20%, 30%, 40%, 50% 60%, 70%, 80%, 90%, 95%, 100%, 2-fold, 3-fold, 4-fold, 5-fold or more decrease in motor neuron degeneration relative to non-treated control. Motor neuron degeneration can be assessed by for example by assaying oxidative stress or endoplasmic reticulum stress or apoptosis or neuronal death in general.

The term “level” as used herein encompasses the absolute amount of cyclin F, the relative amount or concentration of cyclin F as well as any value or parameter which correlates thereto or can be derived therefrom. For example, the level can be weight, moles, abundance, concentration such as μg/L or a relative amount such as 9/10, ⅘, 7/10, ⅗, ½, ⅖, 3/10, ⅕, 1/10, 1/20, 1/50, 10⁻¹, 10⁻², 10⁻³, 10⁻⁴, 10⁻⁵, 10⁻⁶, 10⁻², 10⁻⁸, 10⁻⁹, 10⁻¹⁰, 10⁻¹¹, 10⁻¹², 10⁻¹³, 10⁻¹⁴ or about 10⁻¹⁵ of a reference or control level. Optionally, the term level includes the level of cyclin F normalized to an internal normalization control, such as the expression of a housekeeping gene. The term “level” as applied to the level of cyclin F includes within its scope the level of a CCNF transcript product (e.g., CCNF mRNA) and/or a CCNF translation product (e.g., cyclin F).

The terms “level” and/or “activity” as used herein further refer to gene expression levels or gene activity. Gene expression can be defined as the utilization of the information contained in a gene by transcription and translation leading to the production of a gene product. The measured “expression level” is an indicator for the amount of transcription or translation product produced.

As used herein, the term “modulate” means to cause or facilitate a qualitative or quantitative change, alteration, or modification in a molecule, a process, pathway, or phenomenon of interest. Without limitation, such change may be an increase, decrease, a change in binding characteristics, or change in relative strength or activity of different components or branches of the process, pathway, or phenomenon.

As used herein, the phrase “motor neuron degeneration” or “degeneration of motor neuron” means a condition of deterioration of motor neurons, wherein the neurons die or change to a lower or less functionally-active form.

The term “neurodegenerative condition” is an inclusive term encompassing acute and chronic conditions, disorders or diseases of the central or peripheral nervous system and is generally caused by or associated with the deterioration of cells or tissues of the nervous system. A neurodegenerative condition may be age-related, or it may result from injury or trauma, or it may be related to a specific disease or disorder. Acute neurodegenerative conditions include, but are not limited to, conditions associated with neuronal cell death or compromise including cerebrovascular insufficiency, focal or diffuse brain trauma, diffuse brain damage, spinal cord injury or peripheral nerve trauma, e.g., resulting from physical or chemical burns, deep cuts or limb severance. Examples of acute neurodegenerative disorders are: cerebral ischemia or infarction including embolic occlusion and thrombotic occlusion, reperfusion following acute ischemia, perinatal hypoxic-ischemic injury, cardiac arrest, as well as intracranial hemorrhage of any type (such as epidural, subdural, subarachnoid and intracerebral), and intracranial and intravertebral lesions (such as contusion, penetration, shear, compression and laceration), as well as whiplash and shaken infant syndrome. Chronic neurodegenerative conditions include, but are not limited to, Alzheimer's disease, diffuse Lewy body disease, progressive supranuclear palsy (Steel-Richardson syndrome), multisystem degeneration (Shy-Drager syndrome), chronic epileptic conditions associated with neurodegeneration, motor neuron diseases including amyotrophic lateral sclerosis (ALS), frontotemporal dementia (FTD), degenerative ataxias, cortical basal degeneration, ALS-Parkinson's-Dementia complex of Guam, subacute sclerosing panencephalitis, Huntington's disease, Parkinson's disease, synucleinopathies (including multiple system atrophy), primary progressive aphasia, striatonigral degeneration, Machado-Joseph disease/spinocerebellar ataxia type 3 and olivopontocerebellar degenerations, Gilles De La Tourette's disease, bulbar and pseudobulbar palsy, spinal and spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, familial dysautonomia (Riley-Day syndrome), and prion diseases (including, but not limited to Creutzfeldt-Jakob, Gerstmann-Straussler-Scheinker disease, Kuru and fatal familial insomnia), demyelination diseases and disorders including multiple sclerosis and hereditary diseases such as Leukodystrophies. In specific embodiments, the neurodegenerative condition is selected from ALS and FTD.

As used herein, the term “neuron” includes a neuron and a portion or portions thereof (e.g., the neuron cell body, an axon, or a dendrite). The term “neuron” as used herein denotes nervous system cells that include a central cell body or soma, and two types of extensions or projections: dendrites, by which, in general, the majority of neuronal signals are conveyed to the cell body, and axons, by which, in general, the majority of neuronal signals are conveyed from the cell body to effector cells, such as target neurons or muscle. Neurons can convey information from tissues and organs into the central nervous system (afferent or sensory neurons) and transmit signals from the central nervous systems to effector cells (efferent or motor neurons). Other neurons, designated interneurons, connect neurons within the central nervous system (the brain and spinal column). The neurons may be any neurons, including without limitation sensory, sympathetic, parasympathetic, or enteric, e.g., dorsal root ganglia neurons, motor neurons, and central neurons, e.g., neurons from the spinal cord. Certain specific examples of neuron types that may be subject to treatment or methods according to the present disclosure include cerebellar granule neurons, dorsal root ganglion neurons, and cortical neurons. In some embodiments, the neuron is a sensory neuron. In some embodiments, the neuron is a motor neuron.

The terms “neuron degeneration” and “neuronal degeneration” are used interchangeably herein to refer to any pathological changes in neuronal cells, including, without limitation, death or loss of neuronal cells, any changes that precede cell death, and any reduction or loss of an activity or a function of the neuronal cells. The pathological changes may be spontaneous or may be induced by any event and include, for example, pathological changes associated with apoptosis. The neurons may be any neurons, including without limitation sensory, sympathetic, parasympathetic, or enteric, e.g., dorsal root ganglia neurons, motor neurons, and central neurons, e.g., neurons from the spinal cord. Neuronal degeneration or cell loss is a characteristic of a variety of neurological diseases or disorders, e.g., neurodegenerative diseases or disorders. In some embodiments, the neuron is a sensory neuron. In some embodiments, the neuron is a motor neuron.

The term “neurotropic viral vector” refers to a viral vector that selectively infects neuronal cells, including motor neurons.

By “obtained” is meant to come into possession. Samples so obtained include, for example, nucleic acid extracts or polypeptide extracts isolated or derived from a particular source. For instance, the extract may be isolated directly from a biological fluid or tissue of a subject.

The term “operably connected” or “operably linked” as used herein refers to a juxtaposition wherein the components so described are in a relationship permitting them to function in their intended manner. For example, a regulatory sequence (e.g., a promoter) “operably linked” to a nucleotide sequence of interest (e.g., a coding and/or non-coding sequence) refers to positioning and/or orientation of the control sequence relative to the nucleotide sequence of interest to permit expression of that sequence under conditions compatible with the control sequence. The control sequences need not be contiguous with the nucleotide sequence of interest, so long as they function to direct its expression. Thus, for example, intervening non-coding sequences (e.g., untranslated, yet transcribed, sequences) can be present between a promoter and a coding sequence, and the promoter sequence can still be considered “operably linked” to the coding sequence.

The terms “overexpress,” “overexpression,” or “overexpressed” interchangeably refer to a gene that is transcribed or translated at a detectably greater level in comparison to a normal cell or comparison cell (e.g., a normal motor neuron). Overexpression therefore refers to both overexpression of protein and RNA (due to increased transcription, post transcriptional processing, translation, post translational processing, altered stability, and altered protein degradation), as well as local overexpression due to altered protein traffic patterns (increased nuclear localization), and augmented functional activity, e.g., as in an increased enzyme hydrolysis of substrate. Overexpression can also be at least 10%, 20%, 30%, 40%, 50%, 60%, 70%, 80%, 90% greater in comparison to a normal cell or comparison cell (e.g., a normal motor neuron).

The terms “patient”, “subject”, “host” or “individual” used interchangeably herein, refer to any subject, particularly a vertebrate subject, and even more particularly a mammalian subject, for whom therapy or prophylaxis is desired. Suitable vertebrate animals that fall within the scope of the present disclosure include, but are not restricted to, any member of the subphylum Chordata including primates (e.g., humans, monkeys and apes, and includes species of monkeys such from the genus Macaca (e.g., cynomologus monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards etc.), and fish. A preferred subject is a human in need of increasing the level or activity of cyclin F and/or treatment of a neurodegenerative condition. However, it will be understood that the aforementioned terms do not imply that symptoms are present.

As used here, the term “pharmaceutically acceptable refers to those compounds, agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject agent from one organ, or portion of the body, to another organ, or portion of the body. Each carrier must be “acceptable” in the sense of being compatible with the other ingredients of the formulation and not injurious to the patient.

The term “polynucleotide” is used herein interchangeably with “nucleic acid” to indicate a polymer of nucleosides. Typically, a polynucleotide of present disclosure is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However, the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (e.g., the succession of letters used as abbreviations for bases) that biochemically characterizes a specific nucleic acid. A polynucleotide sequence presented herein is presented in a 5′ to 3′ direction unless otherwise indicated.

The terms “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a non-polypeptide moiety covalently or non-covalently associated therewith is still considered a “polypeptide”. Exemplary modifications include glycosylation and palmitoylation. Polypeptides may be purified from natural sources, produced using recombinant DNA technology, synthesized through chemical means such as conventional solid phase peptide synthesis, etc. The term “polypeptide sequence” or “amino acid sequence” as used herein can refer to the polypeptide material itself and/or to the sequence information (e.g., the succession of letters or three letter codes used as abbreviations for amino acid names) that biochemically characterizes a polypeptide. A polypeptide sequence presented herein is presented in an N-terminal to C-terminal direction unless otherwise indicated.

The term “promoter” refers to a nucleotide sequence, usually upstream (5′) to a transcribable sequence, which controls the expression of the transcribable sequence by providing the recognition for RNA polymerase and other factors required for proper transcription. “Promoter” includes a minimal promoter that is a short nucleic acid sequence comprised of a TATA-box and other sequences that serve to specify the site of transcription initiation, to which control elements (e.g., cis-acting elements) are added for control of expression. “Promoter” also refers to a nucleotide sequence that includes a minimal promoter plus control elements (e.g., ds-acting elements) that are capable of controlling the expression of a coding sequence or functional RNA. This type of promoter sequence consists of proximal and more distal upstream elements, the latter elements often referred to as enhancers. Accordingly, an “enhancer” is a nucleic acid sequence which can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific nucleic acid-binding proteins that mediate their effects. Promoters may be derived in their entirety from a native gene, or be composed of different elements derived from different promoters found in nature, or even be comprised of synthetic nucleic acid segments. A promoter may also contain nucleic acid sequences that are involved in the binding of protein factors which control the effectiveness of transcription initiation in response to physiological or developmental conditions. Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator. The term “regulated promoter” refers to promoters that direct gene expression not constitutively, but in a temporally- and/or spatially-regulated manner, and include both tissue-specific and inducible promoters. It includes natural and synthetic sequences as well as sequences which may be a combination of synthetic and natural sequences. Different promoters may direct the expression of a gene in different tissues or cell types, or at different stages of development, or in response to different environmental conditions. New promoters of various types useful in host cells are constantly being discovered. Since in most cases the exact boundaries of regulatory sequences have not been completely defined, nucleic acid fragments of different lengths may have identical promoter activity. Illustrative regulated promoters include but are not limited to safener-inducible promoters, promoters derived from the tetracycline-inducible system, promoters derived from salicylate-inducible systems, promoters derived from alcohol-inducible systems, promoters derived from glucocorticoid-inducible systems, promoters derived from pathogen-inducible systems, promoters derived from carbohydrate inducible systems, promoters derived from hormone inducible systems, promoters derived from antibiotic inducible systems, promoters derived from metal inducible systems, promoters derived from heat shock inducible systems, and promoters derived from ecdysone-inducible systems.

“Regulatory sequences”, “regulatory elements” and the like refer to nucleotide sequences located upstream (5′ non-coding sequences), within, or downstream (3′ non-coding sequences) of a coding sequence, and which influence the transcription, RNA processing or stability, or translation of the associated coding sequence, either directly or indirectly. Regulatory elements include enhancers, promoters, translation leader sequences, introns, Rep recognition element, intergenic regions and polyadenylation signal sequences. They include natural and synthetic sequences as well as sequences which may be a combination of synthetic and natural sequences.

The term “recombinant polynucleotide” as used herein refers to a polynucleotide formed in vitro by the manipulation of nucleic acid into a form not normally found in nature. For example, the recombinant polynucleotide may be in the form of an expression vector. Generally, such expression vectors include transcriptional and translational regulatory nucleic acid operably linked to the nucleotide sequence.

By “recombinant polypeptide” is meant a polypeptide made using recombinant techniques, i.e., through the expression of a recombinant polynucleotide.

The term “reduced level” as used herein with respect to the level of cyclin F in a motor neuron refers to any level of cyclin F that is below a median level for an age-matched random population of healthy subjects (e.g., an age-matched random population of 10, 20, 30, 40, 50, 100, or 500 healthy subjects) that do not have a neurodegenerative condition. In specific embodiments, a reduced level of cyclin F corresponds to a cyclin F level that is associated with one or both of the following: (1) abnormal localization of TDP-43 to a cellular compartment (e.g., the cytoplasm and/or nucleus); and (2) formation of abnormal TDP-43 structures (e.g., aggregates or inclusions comprising TDP-43). In certain embodiments, a reduced level or activity of cyclin F in a motor neuron is less than about 9/10, ⅘, 7/10, ⅗, ½, ⅖, 3/10, ⅕, 1/10, 1/20, 1/50, 10⁻¹, 10⁻², 10⁻³, 10⁻⁴, 10⁻⁵, 10⁻⁶, 10⁻⁷, 10⁻⁸, 10⁻⁹, 10⁻¹⁰, 10⁻¹¹, 10−12, 10⁻¹³, 10⁻¹⁴ or about 10⁻¹⁵ of the level or activity of cyclin F in a control motor neuron.

The term “sample” as used herein includes any biological specimen that may be extracted, untreated, treated, diluted or concentrated from a subject. Samples may include, without limitation, biological fluids such as whole blood, serum, red blood cells, white blood cells, plasma, saliva, urine, stool (i.e., feces), tears, sweat, sebum, nipple aspirate, ductal lavage, tumor exudates, synovial fluid, ascitic fluid, peritoneal fluid, amniotic fluid, cerebrospinal fluid, lymph, fine needle aspirate, amniotic fluid, any other bodily fluid, cell lysates, cellular secretion products, inflammation fluid, semen and vaginal secretions. Samples may include tissue samples and biopsies, tissue homogenates and the like. In certain embodiments, the sample contains a tissue and in representative examples of this type, the sample is from a resection, biopsy, or core needle biopsy. In addition, fine needle aspirate samples can be used. Samples can include paraffin-embedded and frozen tissue. In specific embodiments, the sample comprises neuronal tissue, including motor neurons. In other embodiments, the sample comprises cells that are surrogates for motor neurons, non-limiting examples of which include fibroblasts, as disclosed for example by Yang et al. (2015, Neurotox Res 28:138-146) and blood cells, as disclosed for example in www.sciencedaily.com/releases/2014/04/140408121918.htm. The term “sample” also includes untreated or pretreated (or pre-processed) samples. In some embodiments, the sample is an untreated biological sample. The sample can be obtained by removing a sample of cells from a subject, but can also be accomplished by using previously isolated cells (e.g., isolated at a prior time point and isolated by the same or another person).

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. The present disclosure contemplates the use in the methods disclosed herein of full-length cyclin F polypeptides as well as their biologically active fragments. Typically, biologically active fragments of a full-length cyclin F polypeptide may participate in an interaction, for example, an intra-molecular or an inter-molecular interaction.

“Similarity” refers to the percentage number of amino acids that are identical or constitute conservative substitutions as defined in Tables 1 and 2 supra. Similarity may be determined using sequence comparison programs such as GAP (Deveraux et al. 1984, Nucleic Adds Research 12: 387-395). In this way, sequences of a similar or substantially different length to those cited herein might be compared by insertion of gaps into the alignment, such gaps being determined, for example, by the comparison algorithm used by GAP.

Terms used to describe sequence relationships between two or more polynucleotides or polypeptides include “reference sequence,” “comparison window”, “sequence identity,” “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

The term “TDP-43 proteinopathy” is used herein to describe neurodegenerative conditions linked to the deposition of TDP-43, including but not limited to amyotrophic lateral sclerosis (ALS), argyrophilic grain disease, frontotemporal dementias (such as FTD-TDP-43 and FTD-tau), ALS-Parkinsonism dementia complex of Guam, corticobasal degeneration, Dementia with Lewy bodies, Huntington's disease (HD), Lewy body disease, motor neuron disease, frontotemporal lobar degeneration (FTLD), frontotemporal dementia, frontotemporal lobar degeneration with ubiquitin-positive inclusions, hippocampal sclerosis, inclusion body myopathy, inclusion body myositis, Parkinson's disease (PD), Parkinson's disease dementia, Parkinson-dementia complex in Kii peninsula, Pick's disease, Machado-Joseph disease and the like. Further details of TDP-43 proteinopathies are described in Gendron et al., 2010, Neuropathol. Appl. Neurobiol. 36:97-112 and Lagier-Tourenne et al., 2010, Hum. Mol. Gen. 19(1):R46-R64; the disclosures of which are incorporated herein by reference. In specific embodiments, the TDP-43 proteinopathy is associated with TDP-43 deposition in neurons, which referred to herein as a “neuronal TDP-43 proteinopathy”.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a condition and/or adverse effect attributable to the condition. “Treatment”, as used herein, covers any treatment of a condition in a mammal, particularly in a human, and includes: (a) inhibiting development of the condition in a subject which may be predisposed to the condition but has not yet been diagnosed as having it; (b) inhibiting the condition, i.e., arresting its development; and (c) relieving the condition, i.e., causing regression of the condition. Thus, “treatment of a neurodegenerative condition” includes within its scope delaying or preventing the onset of such a condition (e.g. death of motor neurons), at reversing, alleviating, ameliorating, inhibiting, slowing down or stopping the progression, aggravation or deterioration the progression or severity of such a condition. In one embodiment, the symptom of a neurodegenerative condition is alleviated by at least 20%, at least 30%, at least 40%, or at least 50%. In one embodiment, the symptom of a neurodegenerative condition is alleviated by more than 50%. In one embodiment, the symptom of a neurodegenerative condition is alleviated by 80%, 90%, or greater. Treatment also includes improvements in neuromuscular function. In some embodiments, neuromuscular function improves by at least about 10%, 20%, 30%, 40%, 50% or more.

The term “transgene” as used herein, refers to any nucleotide sequence used in the transformation of a plant, animal, or other organism. Thus, a transgene can be a coding sequence, a non-coding sequence, a cDNA, a gene or fragment or portion thereof, a genomic sequence, a regulatory element and the like. A “transgenic” organism, such as a transgenic plant, transgenic microorganism, or transgenic animal, is an organism into which a transgene has been delivered or introduced and the transgene can be expressed in the transgenic organism to produce a product, the presence of which can impart an effect and/or a phenotype in the organism.

By “vector” is meant a polynucleotide molecule, suitably a DNA molecule derived, for example, from a plasmid, bacteriophage, yeast or virus, into which a polynucleotide can be inserted or cloned. A vector may contain one or more unique restriction sites and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, i.e., a vector that exists as an extra-chromosomal entity, the replication of which is independent of chromosomal replication, e.g., a linear or closed circular plasmid, an extra-chromosomal element, a mini-chromosome, or an artificial chromosome. The vector can contain any means for assuring self-replication. Alternatively, the vector can be one which, when introduced into the host cell, is integrated into the genome and replicated together with the chromosome(s) into which it has been integrated. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. In the present case, the vector is preferably a viral or viral-derived vector, which is operably functional in animal and preferably mammalian cells. Non-limiting viral vectors that are useful for the practice of the present disclosure include adeno-associated viral vectors (AAV), lentiviral vectors, adenoviral vectors and herpes simplex viral vectors. The vector can also include a selection marker such as an antibiotic resistance gene that can be used for selection of suitable transformants. Examples of such resistance genes are known to those of skill in the art and include the nptII gene that confers resistance to the antibiotics kanamycin and G418 (Geneticin®) and the hph gene which confers resistance to the antibiotic hygromycin B.

The terms “wild-type”, “native” and “naturally occurring” are used interchangeably herein to refer to a gene or gene product that has the characteristics of that gene or gene product when isolated from a naturally occurring source. A wild type, native or naturally occurring gene or gene product (e.g., a polypeptide) is that which is most frequently observed in a population and is thus arbitrarily designed the “normal” or “wild-type” form of the gene or gene product.

As used herein, underscoring or italicizing the name of a gene shall indicate the gene, in contrast to its protein product, which is indicated by the name of the gene in the absence of any underscoring or italicizing. For example, “cyclin F” shall mean the cyclin F gene, whereas “cyclin F” shall indicate the protein product or products generated from transcription and translation and/or alternative splicing of the “cyclin F” gene.

Each embodiment described herein is to be applied mutatis mutandis to each and every embodiment unless specifically stated otherwise.

### 2. Abbreviations

The following abbreviations are used throughout the application:
MND=motor neuron disease
ALS=amyotrophic lateral sclerosis
FTD=frontotemporal dementia
AD=Alzheimer's disease
HD=Huntington's disease
PD=Parkinson's disease

### 3. Agents and Methods for Modulating Viability of Motor Neurons

The present disclosure demonstrates for the first time that cyclin F localizes to the cytoplasm of neurons, including motor neuro, and selectively targets cytoplasmically localized, pathological insTDP-43 for proteolytic degradation, without significantly interfering with the cell cycle regulatory function of nuclear localized sTDP-43. This finding is significant because it extends the utility of cyclin F-enhancing agents to neurodegenerative diseases associated with neuronal TDP-43 proteinopathy, which were previously thought not be susceptible to treatment with such agents, including sporadic neurodegenerative diseases such as sporadic ALS, FTD and AD, which are not associated with neuronal cyclin F deficiency. Consistent with these findings, the present disclosure provides methods for enhancing neuron survival, inhibiting neuron degeneration, inhibiting abnormal protein accumulation in a neuron and/or treating neurodegenerative conditions (e.g., ALS, FTD, AD, etc.), suitably ones that are associated with neuronal TDP-43 proteinopathy, which comprise contacting the neuron with a cyclin F-enhancing agent that increases the level of cyclin F in the neuron, regardless of the neuron's level or activity of endogenous cyclin F, including embodiments in which the neuron does not have a reduced level or activity of endogenous cyclin F relative to a control.

3.1 Cyclin F-Enhancing Agents

The present disclosure contemplates any agent that enhances or increases the level or activity of cyclin F in a neuron (e.g., a motor neuron), to thereby promote neuron survival, inhibit neuron degeneration, and inhibit abnormal protein accumulation in the neuron. In some embodiments, an agent that enhances the level or activity of cyclin F increases the level or activity of cyclin F in the neuron by at least 5%, 10%, 20%, 30%, 40%, 50%, 60%, 70%, 80%, 90%, or 95% or at least 1, 2, 3, 5, 10, 20, 50, or 100-fold more relative to a control.

3.2 Nucleic Acid Constructs

In specific embodiments, the agent is a nucleic acid construct comprising a CCNF polynucleotide that encodes a cyclin F polypeptide and that is operably connected to a promoter. Any CCNF polynucleotide may be used and suitably comprises a nucleotide sequence corresponding to a wild-type CCNF coding sequence, illustrative examples of which are set forth in SEQ ID NO: 1, 2, 4 and 5 or a sequence corresponding thereto (e.g., a sequence that hybridizes under stringency conditions to any one of the sequences set forth in SEQ ID NO: 1, 2, 4 or 5). In certain embodiments, the coding sequence encodes an amino acid sequence as set forth in SEQ ID NO: 3, 6, or 7, or a sequence corresponding thereto.

The present disclosure also contemplates CCNF allelic variants (same locus), homologs (different locus), and orthologs (different organism) as well as non-naturally-occurring CCNF polynucleotides. CCNF polynucleotides can contain nucleotide substitutions, deletions, inversions and insertions relative to a wild-type CCNF polynucleotide sequence. Variation can occur in either or both the coding and non-coding regions. The variations can produce both conservative and non-conservative amino acid substitutions (as compared in the encoded product). For nucleotide sequences, conservative variants include those sequences that, because of the degeneracy of the genetic code, encode the same amino acid sequence as a reference cyclin F polypeptide sequence. CCNF nucleotide sequences also include synthetically derived nucleotide sequences, such as those generated, for example, using site-directed mutagenesis but which still encode a cyclin F polypeptide. Generally, a CCNF nucleotide sequence will have at least about 80%, 81%, 82%, 83%, 84%, 85%, 86%, 87%, 88%, 89%, 90%, 91%, 92%, 93%, 94%, 95%, 96%, 97%, 98%, 99% or more sequence identity to that particular nucleotide sequence as determined by sequence alignment programs described for example herein using default parameters. In some embodiments, the CCNF nucleotide sequence displays at least about 80%, 81%, 82%, 83%, 84%, 85%, 86%, 87%, 88%, 89%, 90%, 91%, 92%, 93%, 94%, 95%, 96%, 97%, 98%, 99% or more sequence identity to a nucleotide sequence selected from any one of SEQ ID NO: 1, 2, 4 or 5, or their complements.

The present disclosure also contemplates polynucleotides that hybridize to reference CCNF nucleotide sequences, or to their complements, (e.g., SEQ ID NO: 1, 2, 4 or 5, or their complements) under stringency conditions described below. As used herein, the term “hybridizes under medium stringency, high stringency, or very high stringency conditions” describes conditions for hybridization and washing. Guidance for performing hybridization reactions can be found in Ausubel et al., (1998, supra), Sections 6.3.1-6.3.6. Aqueous and non-aqueous methods are described in that reference and either can be used. Reference herein to medium stringency conditions include and encompass from at least about 16% v/v to at least about 30% v/v formamide and from at least about 0.5 M to at least about 0.9 M salt for hybridization at 42° C., and at least about 0.1 M to at least about 0.2 M salt for washing at 55° C. Medium stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at 60-65° C. One embodiment of medium stringency conditions includes hybridizing in 6×SSC at about 45° C., followed by one or more washes in 0.2×SSC, 0.1% SDS at 60° C. High stringency conditions include and encompass from at least about 31% v/v to at least about 50% v/v formamide and from about 0.01 M to about 0.15 M salt for hybridization at 42° C., and about 0.01 M to about 0.02 M salt for washing at 55° C. High stringency conditions also may include 1% BSA, 1 mM EDTA, 0.5 M NaHPO₄ (pH 7.2), 7% SDS for hybridization at 65° C., and (i) 0.2×SSC, 0.1% SDS; or (ii) 0.5% BSA, 1 mM EDTA, 40 mM NaHPO₄ (pH 7.2), 1% SDS for washing at a temperature in excess of 65° C. One embodiment of high stringency conditions includes hybridizing in 6×SSC at about 45° C., followed by one or more washes in 0.2×SSC, 0.1% SDS at 65° C.

In certain embodiments, a cyclin F polypeptide is encoded by a polynucleotide that hybridizes to a disclosed nucleotide sequence under very high stringency conditions. One embodiment of very high stringency conditions includes hybridizing 0.5 M sodium phosphate, 7% SDS at 65° C., followed by one or more washes at 0.2×SSC, 1% SDS at 65° C.

Other stringency conditions are well known in the art and a skilled addressee will recognize that various factors can be manipulated to optimize the specificity of the hybridization. Optimization of the stringency of the final washes can serve to ensure a high degree of hybridization. For detailed examples, see Ausubel et al., (1998, supra) at pages 2.10.1 to 2.10.16 and Sambrook et al., (1989, supra) at sections 1.101 to 1.104.

While stringent washes are typically carried out at temperatures from about 42° C. to 68° C., one skilled in the art will appreciate that other temperatures may be suitable for stringent conditions. Maximum hybridization rate typically occurs at about 20° C. to 25° C. below the Tm for formation of a DNA-DNA hybrid. It is well known in the art that the Tm is the melting temperature, or temperature at which two complementary polynucleotide sequences dissociate. Methods for estimating Tm are well known in the art (see Ausubel et al., (1994, supra) at page 2.10.8). In general, the Tm of a perfectly matched duplex of DNA may be predicted as an approximation by the formula:

Tm=81.5+16.6(log 10M)+0.41(% G+C)−0.63(% formamide)−(600/length)

wherein: M is the concentration of Na+, preferably in the range of 0.01 molar to 0.4 molar; % G+C is the sum of guanosine and cytosine bases as a percentage of the total number of bases, within the range between 30% and 75% G+C; % formamide is the percent formamide concentration by volume; length is the number of base pairs in the DNA duplex. The Tm of a duplex DNA decreases by approximately 1° C. with every increase of 1% in the number of randomly mismatched base pairs. Washing is generally carried out at Tm−15° C. for high stringency, or Tm−30° C. for moderate stringency.

In one example of a hybridization procedure, a membrane (e.g., a nitrocellulose membrane or a nylon membrane) containing immobilized DNA is hybridized overnight at 42° C. in a hybridization buffer (50% deionized formamide, 5×SSC, 5×Denhardt's solution (0.1% ficoll, 0.1% polyvinylpyrrolidone and 0.1% bovine serum albumin), 0.1% SDS and 200 mg/mL denatured salmon sperm DNA) containing labeled probe. The membrane is then subjected to two sequential medium stringency washes (i.e., 2×SSC, 0.1% SDS for 15 min at 45° C., followed by 2×SSC, 0.1% SDS for 15 min at 50° C.), followed by two sequential higher stringency washes (i.e., 0.2×SSC, 0.1% SDS for 12 min at 55° C. followed by 0.2×SSC and 0.1% SDS solution for 12 min at 65-68° C.).

3.3 Delivery Vehicles

The present disclosure also contemplates delivery vehicles for delivering CCNF nucleic acid constructs to a neuron (e.g., a motor neuron), including viral vectors and non-viral vectors.

3.3.1 Viral Vectors

Suitable viral vectors for the practice of the methods disclosed herein include, but are not limited to adeno-associated viral vectors (AAV), lentiviral vectors, adenovirus vectors and herpes simplex viral vectors, and in specific embodiments are neurotropic viral vectors.

Adeno-Associated Virus

The CCNF nucleic acid constructs can be delivered to the cells of the central nervous system, including neurons (e.g., motor neurons) by using an adeno-associated viral vector (AAV vector). The use of AAV vectors to deliver genes into the brain is well known in the art (See. e.g., U.S. Pat. Nos. 8,198,257 and 7,534,613, U.S. patent application Ser. No. 13/881,956, each of which is incorporated by reference).

AAV vectors for delivering a CCNF polynucleotide to a motor neuron are known in the art (See U.S. Pat. No. 7,335,636, incorporated by reference). AAV vectors can be constructed using known techniques to provide at least the operably connected components of control elements including a transcriptional initiation region (e.g., a promoter), a transcriptional termination region and optionally at least one post-transcriptional regulatory sequence. The control elements are selected to be functional in the targeted cell. The resulting construct which contains the operably connected components is typically flanked at the 5′ and 3′ region with functional AAV inverted terminal repeat sequences (ITRs).

The nucleotide sequences of AAV ITR regions are known. The ITR sequences for AAV-2 are described, for example, by Kotin et al. Human Gene Therapy, 5:793-01 (1994); Fields & Knipe, Fundamental Virology, “Parvoviridae and their Replication” (2d ed. 1986). The skilled artisan will appreciate that AAV ITR's can be modified using standard molecular biology techniques (e.g., Green & Sambrook, Molecular Cloning: A Laboratory Manual, (4th ed., 2012)). Accordingly, AAV ITRs used in the vectors of the present disclosure need not have a wild-type nucleotide sequence, and may be altered, e.g., by the insertion, deletion or substitution of nucleotides. Additionally, AAV ITRs may be derived from any of several AAV serotypes, including but not limited to, AAV-1, AAV-2, AAV-3, AAV-4, AAV-5, AAV6, AAV7, AAV8, and AAV9, and the like. Furthermore, the 5′ and 3′ ITRs, which flank a selected nucleotide sequence in an AAV expression vector, need not necessarily be identical or derived from the same AAV serotype or isolate, so long as the ITR's function as intended, i.e., to allow for excision and replication of the bounded nucleotide sequence of interest when AAV rep gene products are present in the cell.

The skilled practitioner can appreciate that regulatory sequences can often be provided from commonly used promoters derived from viruses such as, polyoma, Adenovirus 2, cytomegalovirus and Simian Virus 40. Use of viral regulatory elements to direct expression of the protein can allow for high level constitutive expression of the protein in a variety of host cells. Ubiquitously expressing promoters can also be used include, for example, the early cytomegalovirus promoter (Boshart et al., Cell, 41:521-30 (1985)), herpes virus thymidine kinase promoter (McKnight et al. Cell, 37: 253-62 (1984)), β-actin promoters (e.g., the human β-actin promoter, Ng et al., Molecular Cell Biology, 5:2720-32(1985)), and colony stimulating factor-1 promoter (Ladner et al., EMBO J., 6:2693-98(1987)).

Alternatively, the regulatory sequences of the AAV vector can direct expression of the gene preferentially in a particular cell type, i.e., tissue-specific regulatory elements can be used. Non-limiting examples of tissue-specific promoters which can be used include, central nervous system (CNS) specific promoters such as, neuron-specific promoters (e.g., the neurofilament promoter; Byrne and Ruddle, Proc. Natl. Acad. Sci. USA, 86:5473-77 (1989)) and glial specific promoters (Morii et al., Biochemical & Biophysical Research Communications, 175:185-91 (1991)). In specific embodiments, the promoter is tissue specific and is essentially not active outside the central nervous system, or the activity of the promoter is higher in the central nervous system that in other systems. For example, a promoter specific for the spinal cord, brainstem, (medulla, pons, and midbrain), cerebellum, diencephalon (thalamus, hypothalamus), telencephalon (corpus striatum, cerebral cortex, or within the cortex, the occipital, temporal, parietal or frontal lobes), or a combination thereof may be selected. The promoter may be specific for particular cell types, such as neurons or glial cells in the CNS. If it is active in glial cells, it may be specific for astrocytes, oligodendrocytes, ependymal cells, Schwann cells, or microglia. If it is active in neurons, it may be specific for particular types of neurons, e.g., motor neurons, sensory neurons, or interneurons. Additionally, it may be specific for neurons with a specific phenotype, e.g., dopamine-producing neurons, serotonin-producing neurons, etc. In certain embodiments, the promoter is specific for cells in particular regions of the brain, for example, the cortex, striatum, nigra, and hippocampus.

Suitable neuronal specific promoters include, but are not limited to, neuron specific enolase (NSE) (Olivia et al., Genomics, 10:157-65 (1991). GenBank Accession No: X51956), and human neurofilament light chain promoter (NEFL) (Rogaev et al., Human Molecular Genetics, 1:781(1992), GenBank Accession No: L04147). Glial specific promoters include, but are not limited to, glial fibrillary acidic protein (GFAP) promoter (Morii et al., Biochemical & Biophysical Research Communications, 175:185-91 (1991), GenBank Accession No: M65210), 5100 promoter (Morii et al., Biochemical & Biophysical Research Communications, 175:185-91 (1991), GenBank Accession No: M65210) and glutamine synthase promoter (Van den et al., Biochimica Biophysica Acta, 2:249-51(1991), GenBank Accession No: X59834). In a preferred embodiment, the gene is flanked upstream (i.e., 5′) by the neuron specific enolase (NSE) promoter. In another preferred embodiment, the gene of interest is flanked upstream (i.e., 5′) by the elongation factor 1 alpha (EF) promoter. Suitable phenotype-specific promoters include, but are not limited to, tyrosine hydroxylase promoter, dopamine beta-hydroxylase, acetylcholinesterase promoter, choline acetyltransferase promoter, dopamine receptor I and II promoters, dopamine transporter promoter, vesicular monoamine transporter promoter, neuopsin promoter, and vesicular acetylcholine transporter promoter.

The AAV vector harboring a nucleic acid construct from a CCNF polynucleotide is expressible, and a post-transcriptional regulatory sequence (PRE) flanked by AAV ITRs, can be constructed by directly inserting the nucleotide sequence of interest and the PRE into an AAV genome which has had the major AAV open reading frames (“ORFs”) excised therefrom. Other portions of the AAV genome can also be deleted, as long as a sufficient portion of the ITRs remain to allow for replication and packaging functions. These constructs can be designed using techniques well known in the art. (See, e.g., Lebkowski et al., Molecular & Cellular Biology, 8:3988-96 (1988); Vincent et al., Vaccines 90 (Cold Spring Harbor Laboratory Press, 1990); Carter, Current Opinion Biotechnology, 3:533-39 (1992); Muzyczka, Current Topics Microbiology & Immunology, 158:97-29 (1992); Kotin, Human Gene Therapy, 5:793-01(1994); Shelling et al., Gene Therapy, 1:165-69 (1994); and Zhou et al., J Experimental Medicine, 179:1867-75 (1994)). Alternatively, AAV ITRs can be excised from the viral genome or from an AAV vector containing the same and fused 5′ and 3′ of a selected nucleic acid construct that is present in another vector using standard ligation techniques, such as those described in Green & Sambrook (Green & Sambrook, Molecular Cloning: A Laboratory Manual, (4th ed., 2012)). Several AAV vectors are available from the American Type Culture Collection (“ATCC”) under Accession Numbers 53222, 53223, 53224, 53225 and 53226.

In order to produce recombinant AAV particles, an AAV vector can be introduced into a suitable host cell using known techniques, such as by transfection. A number of transfection techniques are generally known in the art (See, e.g., Graham et al., Virology, 52:456 (1973); Green & Sambrook, Molecular Cloning: A Laboratory Manual, (4th ed., 2012); Davis et al., Basic Methods Molecular Biology, (Elsevier, 1986); and Chu et al., Gene, 13:197 (1981)). Particularly suitable transfection methods include calcium phosphate co-precipitation (Graham et al., Virology, 52:456-67 (1973)), direct microinjection into cultured cells (Capecchi, Cell, 22:479-88 (1980)), electroporation (Shigekawa et al., BioTechniques, 6:742-51 (1988)), liposome mediated gene transfer (Mannino et al., BioTechniques, 6:682-90 (1988)), lipid-mediated transduction (Feigner et al., Proceedings Nat'l Acad. Sci. USA, 84:7413-17(1987)), and nucleic acid delivery using high-velocity microprojectiles (Klein et al., Nature 327:70-73 (1987)).

Suitable host cells for producing recombinant AAV particles include, but are not limited to, microorganisms, yeast cells, insect cells, and mammalian cells, that can be, or have been, used as recipients of a exogenous nucleic acid molecule. Thus, a “host cell” as used herein generally refers to a cell which has been transfected with an exogenous nucleic acid molecule. The host cell includes any eukaryotic cell or cell line so long as the cell or cell line is not incompatible with the protein to be expressed, the selection system chosen or the fermentation system employed. Non-limiting examples include CHO DHFR-minus cells (Urlaub and Chasin Proceedings Nat'l Acad. Sci. USA, 77:4216-420 (1980)), 293 cells (Graham et al., J. General Virology 36:59-72 (1977)), or myeloma cells like SP2 or NSO (Galfre & Milstein, Methods Enzymology, 73:3-46 (1981)).

In some embodiments, the host cells are cells from the stable human cell line, 293 (readily available through, e.g., the ATCC under Accession No. ATCC CRL 1573), which is a human embryonic kidney cell line that has been transformed with adenovirus type-5 DNA fragments (Graham et al., J. General Virology, 36:59-72 (1977)), and expresses the adenoviral E1a and E1b genes (Aiello et al., Virology, 94:460-69 (1979)). The 293 cell line is readily transfected, and provides a particularly convenient platform in which to produce AAV virions.

Host cells containing the above-described AAV vectors must be rendered capable of providing AAV helper functions in order to replicate and encapsidate the expression cassette flanked by the AAV ITRs to produce recombinant AAV particles. AAV helper functions are generally AAV-derived coding sequences which can be expressed to provide AAV gene products that, in turn, function in trans for productive AAV replication. AAV helper functions are used herein to complement necessary AAV functions that are missing from the AAV vectors. Thus. AAV helper functions include one, or both of the major AAV open reading frames (ORFs), namely the rep and cap coding regions, or functional homologues thereof.

The AAV rep coding region of the AAV genome encodes the replication proteins Rep 78, Rep 68, Rep 52, and Rep 40. These Rep expression products have been shown to possess many functions, including recognition, binding and nicking of the AAV origin of DNA replication, DNA helicase activity and modulation of transcription from AAV (or other exogenous) promoters. The Rep expression products are collectively required for replicating the AAV genome. The AAV cap coding region of the AAV genome encodes the capsid proteins VP1, VP2, and VP3, or functional homologues thereof. AAV helper functions can be introduced into the host cell by transfecting the host cell with an AAV helper construct either prior to, or concurrently with, the transfection of the AAV vector comprising the expression cassette, AAV helper constructs are thus used to provide at least transient expression of AAV rep and/or cap genes to complement missing AAV functions that are necessary for productive AAV infection. AAV helper constructs lack AAV ITRs and can neither replicate nor package themselves. These constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products (See, e.g., Samulski et al., J. Virology, 63:3822-28 (1989); McCarty et al., J. Virology, 65:2936-45 (1991)). A number of other vectors have been described which encode Rep and/or Cap expression products (See. e.g., U.S. Pat. No. 5,139,941, incorporated by reference).

As a consequence of the infection of the host cell with a helper virus, the AAV Rep and/or Cap proteins are produced. The Rep proteins also serve to duplicate the AAV genome. The expressed Cap proteins assemble into capsids, and the AAV genome is packaged into the capsids. This results the AAV being packaged into recombinant AAV particles comprising the expression cassette. Following recombinant AAV replication, recombinant AAV particles can be purified from the host cell using a variety of conventional purification methods, such as CsCl gradients. The resulting recombinant AAV particles are then ready for use for gene delivery to various cell types.

In some embodiments, the number of viral vector and/or virion particles administered to a subject may be on the order ranging from 10³ to 10¹⁵ particles/mL, or any values therebetween, such as for example, about 10⁷, 10⁸, 10⁹, 10¹⁰, 10¹¹, 10¹², 10¹³, 10¹⁴, or 10¹⁵ particles/mL. In some embodiments, vector and/or virion particles of higher than 10¹³ particles/mL are administered. Volumes between 1 μL and 10 mL may be administered such that the subject receives between 10² and 10¹⁶ total vector and/or virion particles. Thus, in some embodiments, about 10², 10³, 10⁴, 10⁵, 10⁶, 10⁷, 10⁸, 10⁹, 10¹⁰, 10¹¹, 10¹², 10¹³, 10¹⁴, 10¹⁴ or 10¹⁶ vector and/or virion particles are administered.

In the practice of the methods of the present disclosure, an AAV of any serotype can be used. The serotype of the viral vector used in certain embodiments of the invention is selected from the group consisting from AAV1, AAV2, AAV3, AAV4, AAV5, AAV6, AAV7, and AAV8 (see, e.g., Gao et al., 2002, PNAS 99:11854-11859; and Viral Vectors for Gene Therapy: Methods and Protocols, ed. Machida, Humana Press, 2003). Other serotypes besides those listed herein can be used. Furthermore, pseudotyped AAV vectors may also be utilized in the methods described herein. Pseudotyped AAV vectors are those which contain the genome of one AAV serotype in the capsid of a second AAV serotype; for example, an AAV vector that contains the AAV2 capsid and the AAV1 genome or an AAV vector that contains the AAV5 capsid and the AAV 2 genome (Auricchio et al., 2001. Hum. Mol. Genet. 10(26):3075-81). AAV vectors are derived from single-stranded (ss) DNA parvoviruses that are nonpathogenic for mammals (reviewed in Muzyscka, 1992. Curr. Top. Microb. Immunol. 158:97-129). Briefly, recombinant AAV-based vectors have the rep and cap viral genes that account for 96% of the viral genome removed, leaving the two flanking 145-basepair (bp) inverted terminal repeats (ITRs), which are used to initiate viral DNA replication, packaging and integration. In the absence of helper virus, wild-type AAV integrates into the human host-cell genome with preferential site-specificity at chromosome 19q 13.3 or it may be maintained episomally. A single AAV particle can accommodate up to 5 kb of ssDNA, therefore leaving about 4.5 kb for a transgene and regulatory elements, which is typically sufficient. However, trans-splicing systems as described, for example, in U.S. Pat. No. 6,544,785, may nearly double this limit.

In certain instances, the AAV serotype is selected from AAV1, AAV2, AAV3, AAV4, AAV5, AAV6, AAV6.2, AAV7, AAV8, AAV9, rh.10, rh.39, rh.43, and CSp3.

AAV-based gene therapy vectors targeted to cells of the CNS have been described for example in U.S. Pat. Nos. 6,180,613 and 6,503,888. Additional exemplary AAV vectors are recombinant AAV2/1, AAV2/2, AAV2/5, AAV2/7, AAV2/8 and AAV2/9 serotype vectors encoding human protein. In specific embodiments, the AAV is a neurotropic AAV selected from rAAV2/1, rAAV2/8 and rAAV2/9, as described for example in Ayers et al. (2015, Mol Ther. 23(1): 53-62).

Alternatively, a vector of the present disclosure can be a virus other than the adeno-associated virus, or portion thereof, which allows for expression of a CCNF nucleic acid molecule introduced into the viral nucleic acid. For example, replication defective retroviruses, adenoviruses, herpes simplex viruses, and lentivirus can be used. Protocols for producing recombinant retroviruses and for infecting cells in vitro or in vivo with such viruses can be found in Ausubel et al., Current Protocols in Molecular Biology §§ 9.10-9.14 (Greene Publishing Associates, 1989) and other standard laboratory manuals. Examples of suitable retroviruses include pU, pZIP, pWE and pEM, which are well known to those skilled in the art. Examples of suitable packaging virus lines include Crip, Cre, 2 and Am. The genome of adenovirus can be manipulated such that it encodes and expresses the protein of interest but is inactivated in terms of its ability to replicate in a normal lytic viral life cycle (See e.g., Berkner et al., BioTechniques, 6:616-29 (1988); Rosenfeld et al., Science, 252:431-34 (1991); Rosenfeld et al., Cell 68:143-55 (1992)). Suitable adenoviral vectors derived from the adenovirus strain Ad type 5 d1324 or other strains of adenovirus (e.g., Ad2, Ad3, Ad7 etc.) are well known to those skilled in the art.

Lentivirus

Lentiviral vectors may be utilized to express CCNF polynucleotides in cell of the nervous system, including neurons (e.g., motor neurons), and the production of suitable lentiviral vectors is well known in the art (See, e.g., U.S. patent application Ser. No. 13/893,920, incorporated by reference). The lentiviral vector according to the present disclosure may be derived from or may be derivable from any suitable lentivirus. A recombinant lentiviral particle is capable of transducing a target cell with a nucleotide of interest. Once within the cell the RNA genome from, the vector particle is reverse transcribed into DNA and integrated into the genome of the target cell.

Lentiviral vectors are part of a larger group of retroviral vectors. A detailed list of lentiviruses may be found in Coffin et al., Retroviruses 758-763 (Cold Spring Harbor Laboratory Press, 1997). In brief, lentiviruses can be divided into primate and non-primate groups. Examples of primate lentiviruses include but are not limited to the human immunodeficiency virus (HIV) and the simian immunodeficiency virus (SrV). The non-primate lentiviral group includes the prototype “slow virus” visnaimaedi virus (VMV), as well as the related caprine arthritis-encephalitis virus (CAEV), equine infectious anemia virus (EIAV), feline immunodeficiency virus (FIV), bovine immunodeficiency virus (BIV).

Lentiviruses differ from other members of the retrovirus family in that lentiviruses have the capability to infect both dividing and non-dividing cells (Lewis et al., EMBO J., 11:3053-58 (1992)); Lewis & Emerman, J Virology, 68:510-16 (1994)). In contrast, other retroviruses, such as MLV, are unable to infect non-dividing or slowly dividing cells such as those that make up, for example, muscle, brain, lung and liver tissue.

A lentiviral vector, as used herein, is a vector which comprises at least one component part derivable from a lentivirus. That component part may be involved in the biological mechanisms by which the vector infects cells, expresses genes or is replicated. The basic structure of retrovirus and lentivirus genomes share many common features such as a 5′ LTR and a 3′ LTR, between or within which are located a packaging signal to enable the genome to be packaged, a primer binding site, integration sites to enable integration into a host cell genome and gag, pol and env genes encoding the packaging components, which are polypeptides required for the assembly of viral particles. Lentiviruses have additional features, such as the rev and rev response element (RRE) sequences, which enable the efficient export of RNA transcripts of the integrated provirus from the nucleus to the cytoplasm of an infected target cell. In the provirus, the viral genes are flanked at both ends by regions called long terminal repeats (LTRs). The LTRs are responsible for proviral integration, and transcription. LTRs also serve as enhancer-promoter sequences and can control the expression of the viral genes. The LTRs themselves are identical sequences that can be divided into three elements, which are called “U3,” “R” and “U5.” U3 is derived from the sequence unique to the 3′ end of the RNA, R is derived from a sequence repeated at both ends of the RNA, and U5 is derived from the sequence unique to the 5′ end of the RNA. The sizes of the three elements can vary considerably among different viruses.

In a defective lentiviral vector genome gag, pol and env may be absent or non-functional. The R regions at both ends of the RNA are repeated sequences. U5 and U3 represent unique sequences at the 5′ and 3′ ends of the RNA genome respectively.

In a typical lentiviral vector of the present disclosure, at least part of one or more protein coding regions essential for replication may be removed from the virus, which makes the viral vector replication-defective. Portions of the viral genome may also be replaced by a nucleic acid in order to generate a vector comprising the nucleic acid which is capable of transducing a target non-dividing host cell and/or integrating its genome into a host genome. In one embodiment, the lentiviral vectors are non-integrating vectors as described in U.S. patent application Ser. No. 12/138,993 (herein incorporated by reference).

In a further embodiment, the vectors have the ability to deliver a sequence which is devoid of or lacking viral RNA. A heterologous binding domain (heterologous to gag) may be located on the RNA to be delivered and a cognate binding domain on gag or pol can be used to ensure packaging of the RNA to be delivered. Both of these vectors are described in U.S. patent application Ser. No. 12/139,035 (herein incorporated by reference). The lentiviral vector may be a “non-primate” vector, i.e., derived from a virus which does not primarily infect primates, especially humans.

The examples of non-primate lentivirus may be any member of the family of Lentiviridae which does not naturally infect a primate and may include a feline immunodeficiency virus (FIV), a bovine immunodeficiency virus (BIV), a caprine arthritis encephalitis virus (CAEV), a Maedi-visna virus (MW) or an equine infectious anemia virus (EIAV).

In some embodiments, the viral vector is derived from EIAV. EIAV has the simplest genomic structure of the lentiviruses. In addition to the gag, pol and env genes, EIAV encodes three other genes: tat, rev, and S2. Tat acts as a transcriptional activator of the viral LTR (Derse & Newbold, Virology, 194:530-36(1993); Maury et al., Virology, 200:632-42(1994)). Rev regulates and coordinates the expression of viral genes through rev-response elements (RRE) (Martarano et al., J. Virology, 68:3102-11 (1994)). The mechanisms of action of these two proteins are thought to be broadly similar to the analogous mechanisms in the primate viruses (Martarano et al., J. Virology, 68:3102-11 (1994)). The function of S2 is unknown. In addition, an EIAV protein, Ttm, has been identified that is encoded by the first exon of tat spliced to the env coding sequence at the start of the transmembrane protein.

The viral vector may be manipulated to remove the non-essential elements and to retain the essential elements in order to provide the required functionality to infect, transduce and deliver a nucleotide sequence of interest to a target host cell (See, e.g., U.S. Pat. No. 6,669,936, incorporated by reference). In some embodiments, the genome is limited to sufficient lentiviral genetic information to allow packaging of an RNA genome, in the presence of packaging components, into a viral particle capable of infecting a target cell. Infection of the target cell may include reverse transcription and integration into the target cell genome. The lentiviral vector carries non-viral coding sequences which are to be delivered by the vector to the target cell. In some embodiments, the vector is incapable of independent replication to produce infectious lentiviral particles within the final target cell. Usually the recombinant lentiviral vector lacks a functional gag-pol and/or env gene and/or other genes essential for replication. A vector of the present disclosure may be configured as a split-intron vector (See, e.g., U.S. Pat. No. 7,303,910, incorporated by reference).

The vector may be a self-inactivating vector. Self-inactivating retroviral vectors may be constructed by deleting the transcriptional enhancers or the enhancers and promoter in the U3 region of the 3′ LTR. After a round of vector reverse transcription and integration, these changes are copied into both the 5′ and the 3′ LTRs producing a transcriptionally inactive provirus (Yu et al., Proceedings Nat'l Acad. Sci. USA, 83:3194-98 (1986); Dougherty and Temin et al., Proceedings Nat'l Acad. Sci. USA, 84:1197-01 (1987): Hawley, Proceedings Nat'l Acad. Sci. USA, 84:2406-10 (1987); Yee et al., Proceedings Nat'l Acad. Sci. USA, 91:9564-68 (1994)). However, any promoter(s) internal to the LTRs in such vectors will still be transcriptionally active. This strategy has been employed to eliminate effects of the enhancers and promoters in the viral LTRs on transcription from internally placed genes. Such effects include increased transcription (Jolly et al., Nucleic Acids Research, 11:1855-72 (1983)) or suppression of transcription (Emerman & Temin, Cell, 39:449-67 (1984)). This strategy can also be used to eliminate downstream transcription from the 3′ LTR into genomic DNA (Herman & Coffin, Science, 236:845-48 (1987)). This is of particular concern in human gene therapy where it is of critical importance to prevent the adventitious activation of an endogenous oncogene.

The plasmid vector used to produce the viral genome within a host cell/packaging cell will also include transcriptional regulatory control sequences operably linked to the lentiviral genome to direct transcription of the genome in a host cell/packaging cell. These regulatory sequences may be the natural sequences associated with the transcribed lentiviral sequence, i.e. the 5′ U3 region, or they may be a heterologous promoter such as another viral promoter, for example the CMV promoter. Some lentiviral genomes require additional sequences for efficient virus production. For example, in the case of HIV, the rev and RRE sequences are preferably included; however the requirement for rev and RRE may be reduced or eliminated by codon optimization (See U.S. patent application Ser. No. 12/587,236, incorporated by reference). Alternative sequences which perform the same function, as the rev/RRE system are also known. For example, a functional analogue of the revIRRE system is found in the Mason Pfizer monkey virus. This is known as the constitutive transport element (CTE) and comprises an RRE-type sequence in the genome which is believed to interact with a factor in the infected cell. The cellular factor can be thought of as a rev analogue. Thus, CTE may be used as an alternative to the reviRRE system. Any other functional equivalents which are known or become available may be relevant to the methods of the present disclosure. For example, the Rex protein of HTLV-1 can functionally replace the Rev protein of HIV-1. It is also known that Rev and Rex have similar effects to IRE-BP.

In certain embodiments, the lentiviral vector is a self-inactivating minimal lentiviral vector, derived from Equine Infectious Anemia Virus (EIAV), from which a CCNF polynucleotide is expressible. The vector may be produced by the transient transfection of cells (e.g. HEK293T cells) with three plasmids, encoding for: (1) the recombinant EIAV PROSAVIN (Oxford BioMedica pic, Oxford UK) vector genome (Farley et al., J. Gen. Med., 9:345-56 (2007); U.S. Pat. No. 7,259,015, incorporated by reference); (2) the synthetic EIAV gag/pol expression vector (pESGPK, U.S. patent application Ser. Nos. 13/893,920 and 12/587,236, incorporated by reference) and (3) the VSV-G envelope expression vector (pHGK).

Herpes Simplex Virus

Herpes simplex virus (HSV) vectors may also be utilized to express a CCNF polynucleotide in cells of the nervous system, including neurons (e.g., motor neurons). The genome of the type-1 (HSV-1) is about 150 kb of linear, double-stranded DNA, featuring about 70 genes. Many viral genes may be deleted without the virus losing its ability to propagate. The “immediately early” (IE) genes are transcribed first. They encode trans-acting factors which regulate expression of other viral genes. The “early” (E) gene products participate in replication of viral DNA. The late genes encode the structural components of the virion as well as proteins that turn on transcription of the IE and E genes or disrupt host cell protein translation.

The HSV vector may be a plasmid-based system, whereby a plasmid vector (termed an amplicon) is generated that contains a nucleotide sequence encoding the gene and two cis-acting HSV recognition signals. The recognition signals are the origin of DNA replication and the cleavage packaging signal, which encode no HSV gene products. Thus, helper virus is required to replicate the amplicon and package it into an HSV coat. The vector therefore expresses no viral gene products within the recipient cell, and recombination with or reactivation of latent viruses by the vector is limited due to the minimal amount of HSV DNA sequence present within the defective HSV vector genome.

Examples of HSV-mediated gene therapy are well known in the art (Breakefield & DeLuca. New Biologist, 3:203-18 (1991); Ho & Mocarski, Virology, 167:279-93 (1988); Palella, et al., Molecular & Cellular Biology, 8:457-60 (1988); Palella et al., Gene, 80:137-44 (1988); Andersen et al., Human Gene Therapy, 3:487-99 (1992); Kaplitt et al., Current Topics Neuroendocrinology, 11:169-91 (1993); Spade & Frenkel, Cell, 30:295-04 (1982); Kaplitt et al., Molecular & Cellular Neuroscience, 2:320-30 (1991); Federoff et al., Proceedings Nat. Acad. Sci. USA, 89:1636-40 (1992)).

Adenovirus

Adenovirus vectors may be utilized to express CCNF polynucleotides in cells of the nervous system, including neurons (e.g., motor neurons). The adenovirus genome consists of about 36 kb of double-stranded DNA. Adenoviruses target airway epithelial cells, but are also capable of infecting neurons. Recombinant adenovirus vectors have been used as gene transfer vehicles for non-dividing cells. These vectors are similar to recombinant HSV vectors, since the adenovirus Ela immediate-early gene is removed but most viral genes are retained. Since the Ela gene is small (roughly 1.5 kb) and the adenovirus genome is approximately one-third of the size of the HSV genome, other non-essential adenovirus genes are removed in order to insert a foreign gene within the adenovirus genome.

Examples of adenovirus-mediated gene therapy are well known in the art (Akli et al., Nature Genetics, 3:224-28 (1993); La Salle et al., Science, 259:988-90 (1993), La Salle, Nature Genetics, 3:1-2 (1993); Neve, Trends Biochemical Sci., 16:251-53 (1993)).

3.3.2 Non-Viral Vectors

Cyclin F can be delivered using a non-viral delivery system, for example, as naked nucleic acid, in combination with a delivery reagent. Any nucleic acid delivery method known in the art can be used in the methods described herein. This includes delivery of a nucleic acid to the desired tissues in colloidal dispersion systems that include, for example, macromolecule complexes, nanocapsules, microspheres, beads, and lipid-based systems including oil-in-water emulsions, micelles, mixed micelles, and liposomes. Suitable delivery reagents include, but are not limited to, e.g., the Mirus Transit TKO lipophilic reagent; lipofectin; lipofectamine; cellfectin; polycations (e.g., polylysine), atelocollagen, nanoplexes and liposomes. The use of atelocollagen as a delivery vehicle for nucleic acid molecules is described in Minakuchi et al. Nucleic Acids Research, 32:e109 (2004); Hanai et al. Annals N.Y. Acad. Sci., 1082:9-17 (2006); Kawata et al. Molecular Cancer Therapeutics, 7:2904-12 (2008).

Liposomes are artificial membrane vesicles which are useful as delivery vehicles in vitro and in vivo. In order for a liposome to be an efficient gene transfer vehicle, the following characteristics should be present: (1) encapsulation of the genetic material at high efficiency while not compromising the biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino et al., BioTechniques, 6:682-90 (1988)).

Liposomes suitable for use in the methods described herein can be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. Examples of suitable lipids liposomes production include phosphatidyl compounds, such as phosphatidylglycerol, phosphatidylcholine, phosphatidylserine, phosphatidylethanolamine, sphingolipids, cerebrosides, and gangliosides. Additional examples of lipids include, but are not limited to, polylysine, protamine, sulfate and 3.beta.-[N—(N′,N′-dimethylaminoethane) carbamoyl] cholesterol. A variety of methods are known for preparing liposomes, for example, as described in Szoka et al., Annual Rev. Biophysics & Bioengineering, 9:467-08 (1980); and U.S. Pat. Nos. 4,235,871; 4,501,728; 4,837,028; and 5,019,369, which are herein incorporated by reference.

Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 m. Sonication of MLVs results in the formation of small unilamellar vesicles (SUVs) with diameters in the range of 200 to 500 angstroms, containing an aqueous solution in the core.

The liposomes for use in the present methods can also be modified so as to avoid clearance by the mononuclear macrophage system (“MMS”) and reticuloendothelial system (“RES”). Such modified liposomes have opsonization-inhibition moieties on the surface or incorporated into the liposome structure.

Opsonization-inhibiting moieties for use in preparing the liposomes described herein are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer that significantly decreases the uptake of the liposomes by the MMS and RES; e.g., as described in U.S. Pat. No. 4,920,016, herein incorporated by reference.

In some embodiments, opsonization inhibiting moieties suitable for modifying liposomes are water-soluble polymers with a number-average molecular weight from about 500 to about 40,000 Daltons, or from about 2,000 to about 20,000 Daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups. In some embodiments, the opsonization-inhibiting moiety is a PEG, PPG, or derivatives thereof. Liposomes modified with PEG or PEG-derivatives are sometimes called “PEGylated liposomes.”

### 4. Delivery of Cyclin F-Enhancing Agents

Neurons (e.g., motor neurons) can be contacted with a cyclin F-enhancing agents described herein in a cell culture e.g., in vitro or ex vivo, or administered to a subject, e.g., in vivo. In some embodiments, a cyclin F-enhancing agent described herein can be administered to a subject to treat or inhibit the development of a neurodegenerative conditions, including those that are associated with neuronal TDP-43 proteinopathy, such as ALS, FTD and AD.

For in vitro methods, neurons can be obtained from different sources. For example, neurons can be obtained from a subject. In some embodiments, the neuron is a whole cell. In some embodiments, the subject is suffering from a neurodegenerative condition (e.g., a neurodegenerative condition associated with neuronal TDP-43 proteinopathy). In some embodiments, the subject is at risk of developing a neurodegenerative condition (e.g., a neurodegenerative condition associated with neuronal TDP-43 proteinopathy). In some embodiments, the subject is suspected of having a neurodegenerative condition (e.g., a neurodegenerative condition associated with neuronal TDP-43 proteinopathy). In some embodiments, the subject is at risk of developing a condition characterized by neuronal cell death. In some embodiments, the subject is suspected of suffering from a condition characterized by neuronal cell death. In some embodiments, the subject is suffering from neuronal cell death. In some embodiments, the subject is suffering from ALS. In some embodiments, the subject is suffering from FTD. In some embodiments, the subject is suffering from AD. In some embodiments, the subject is a carrier e.g., a symptom-free carrier. In some embodiments, motor neuron cells are derived from a subject's embryonic stem cells (ESCs). In some embodiments, the subject is human. In some embodiments, the subject is mouse. In some embodiments, the mouse is a transgenic mouse. Methods of inducing motor neuron differentiation from embryonic stem cells are known in the art, for example as described in Di Giorgio et al., Nature Neuroscience (2007), published online 15 Apr. 2007; doi:10.1038/nn1885 and Wichterle et al., Cell (2002) 110:385-397. In some instances, induced pluripotent stem cells can be generated from a subject and then differentiated into motor neurons. One exemplary method of deriving motor neurons from a subject is described in Dimos, J. T., et al. Science (2008) 321, 1218-122 (Epub Jul. 31, 2008).

For in vivo methods, an effective amount of a cyclin F-enhancing agent described herein can be administered to a subject. Methods of administering agents to a subject are known in the art and easily available to one of skill in the art.

Those skilled in the art will also appreciate that the agents described herein can be used for inhibiting neuron degeneration or enhancing neuron survival, which can lead to treatment, inhibition of development or amelioration of a number of conditions characterized by neuron (e.g., motor neuron) degeneration.

In specific embodiments, the neuron degeneration comprises motor neuron degeneration. The motor neuron diseases (MND) are a group of neurodegenerative conditions that selectively affect motor neurons, the nerve cells that control voluntary muscle activity including speaking, walking, breathing, swallowing and general movement of the body. Skeletal muscles are innervated by a group of neurons (lower motor neurons) located in the ventral horns of the spinal cord which project out the ventral roots to the muscle cells. These nerve cells are themselves innervated by the corticospinal tract or upper motor neurons that project from the motor cortex of the brain. On macroscopic pathology, there is a degeneration of the ventral horns of the spinal cord, as well as atrophy of the ventral roots. In the brain, atrophy may be present in the frontal and temporal lobes. On microscopic examination, neurons may show spongiosis, the presence of activated astrocytes and microglia, and a number of inclusions including characteristic “skein-like” inclusions, bunina bodies, and vacuolization. Motor neuron diseases are varied and destructive in their effect. They commonly have distinctive differences in their origin and causation, but a similar result in their outcome for the patient: severe muscle weakness. Amyotrophic lateral sclerosis (ALS), frontotemporal dementia (FTD), primary lateral sclerosis (PLS), progressive muscular atrophy (PMA), pseudobulbar palsy, progressive bulbar palsy, spinal muscular atrophy (SMA) and post-polio syndrome are all examples of MND. The major site of motor neuron degeneration classifies the neurodegenerative condition.

ALS, which affects both upper and lower motor neurons, is the most common form of MND. Progressive bulbar palsy affects the lower motor neurons of the brain stem, causing slurred speech and difficulty chewing and swallowing. Individuals with these conditions almost always have abnormal signs in the arms and legs. Primary lateral sclerosis is a disease of the upper motor neurons, while progressive muscular atrophy affects only lower motor neurons in the spinal cord. Means for diagnosing MND are well known to those skilled in the art. Non limiting examples of symptoms are described below.

4.1 Amyotrophic Lateral Sclerosis (ALS)

Amyotrophic lateral sclerosis (ALS), also called Lou Gehrig's disease or classical motor neuron disease, is a progressive, ultimately fatal disorder that eventually disrupts signals to all voluntary muscles. In the United States, doctors use the terms motor neuron disease and ALS interchangeably. Both upper and lower motor neurons are affected. Approximately 75 percent of people with classic ALS will also develop weakness and wasting of the bulbar muscles (muscles that control speech, swallowing, and chewing). Symptoms are usually noticed first in the arms and hands, legs, or swallowing muscles. Muscle weakness and atrophy occur disproportionately on both sides of the body. Affected individuals lose strength and the ability to move their arms, legs, and body. Other symptoms include spasticity, exaggerated reflexes, muscle cramps, fasciculations, and increased problems with swallowing and forming words. Speech can become slurred or nasal. When muscles of the diaphragm and chest wall fail to function properly, individuals lose the ability to breathe without mechanical support. Although the disease does not usually impair a person's mind or personality, several recent studies suggest that some people with ALS may have alterations in cognitive functions such as problems with decision-making and memory. ALS most commonly strikes people between 40 and 60 years of age, but younger and older people also can develop the disease. Men are affected more often than women. Most cases of ALS occur sporadically, and family members of those individuals are not considered to be at increased risk for developing the disease. However, there is a familial form of ALS in adults, which often results from mutation of genes responsible for RNA metabolism (e.g., TDP43 and FUS) and protein degradation (e.g., UBQLN2, TBK1 and CCNF). In addition, a rare juvenile-onset form of ALS is genetic. Most individuals with ALS die from respiratory failure, usually within 3 to 5 years from the onset of symptoms. However, about 10 percent of affected individuals survive for 10 or more years.

4.2 Frontotemporal Dementia (FTD)

Frontotemporal dementia (FTD) is the clinical presentation of frontotemporal lobar degeneration, which is characterized by progressive neuronal loss predominantly involving the frontal and/or temporal lobes, and typical loss of over 70% of spindle neurons, while other neuron types remain intact In FTD, portions of frontal and temporal lobes atrophy or shrink. The frontal and temporal lobes of the brain are generally associated with personality, behavior and language. Common signs and symptoms vary, depending upon the portion of the brain affected. Some people with FTD undergo dramatic changes in their personality and become socially inappropriate, impulsive or emotionally indifferent, while others lose the ability to use language. signs and symptoms include significant changes in social and personal behavior, apathy, blunting of emotions, and deficits in both expressive and receptive language. Currently, there is no cure for FTD, but there are treatments that help alleviate symptoms.

4.3 Spinal Muscular Atrophy (SMA)

Spinal muscular atrophy (SMA) refers to a number of different disorders, all having in common a genetic cause and the manifestation of weakness due to loss of the motor neurons of the spinal cord and brainstem. Weakness and wasting of the skeletal muscles is caused by progressive degeneration of the anterior horn cells of the spinal cord. This weakness is often more severe in the legs than in the arms. SMA has various forms, with different ages of onset, patterns of inheritance, and severity and progression of symptoms. Some of the more common SMAs are described below.

Defects in SMN gene products are considered as the major cause of SMA and SMN protein levels correlate with survival of subject suffering from SMA. The most common form of SMA is caused by mutation of the SMN gene. The region of chromosome 5 that contains the SMN (survival motor neuron) gene has a large duplication. A large sequence that contains several genes occurs twice in adjacent segments. There are thus two copies of the gene, SMN1 and SMN2. The SMN2 gene has an additional mutation that makes it less efficient at making protein, though it does so in a low level. SMA is caused by loss of the SMN1 gene from both chromosomes. The severity of SMA, ranging from SMA 1 to SMA 3, is partly related to how well the remaining SMN 2 genes can make up for the loss of SMN 1.

SMA type I, also called Werdnig-Hoffmann disease, is evident by the time a child is 6 months old. Symptoms may include hypotonia (severely reduced muscle tone), diminished limb movements, lack of tendon reflexes, fasciculations, tremors, swallowing and feeding difficulties, and impaired breathing. Some children also develop scoliosis (curvature of the spine) or other skeletal abnormalities. Affected children never sit or stand and the vast majority usually die of respiratory failure before the age of 2.

Symptoms of SMA type II usually begin after the child is 6 months of age. Features may include inability to stand or walk, respiratory problems, hypotonia, decreased or absent tendon reflexes, and fasciculations. These children may learn to sit but do not stand. Life expectancy varies, and some individuals live into adolescence or later.

Symptoms of SMA type III (Kugelberg-Welander disease) appear between 2 and 17 years of age and include abnormal gait; difficulty running, climbing steps, or rising from a chair; and a fine tremor of the fingers. The lower extremities are most often affected. Complications include scoliosis and joint contractures—chronic shortening of muscles or tendons around joints, caused by abnormal muscle tone and weakness, which prevents the joints from moving freely.

Other forms of SMA include e.g., Hereditary Bulbo-Spinal SMA Kennedy's disease (X linked, Androgen receptor), SMA with Respiratory Distress (SMARD 1) (chromosome 11, IGHMBP2 gene), Distal SMA with upper limb predominance (chromosome 7, glycyl tRNA synthase), and X-Linked infantile SMA (gene UBE1).

Current treatment for SMA consists of prevention and management of the secondary effect of chronic motor unit loss. Some drugs under clinical investigation for the treatment of SMA include butyrates, Valproic acids, hydroxyurea and Riluzole.

Symptoms of Fazio-Londe disease appear between 1 and 12 years of age and may include facial weakness, dysphagia (difficulty swallowing), stridor (a high-pitched respiratory sound often associated with acute blockage of the larynx), difficulty speaking (dysarthria), and paralysis of the eye muscles. Most individuals with SMA type III die from breathing complications.

Kennedy disease, also known as progressive spinobulbar muscular atrophy, is an X-linked recessive disease. Daughters of individuals with Kennedy disease are carriers and have a 50 percent chance of having a son affected with the disease. Onset occurs between 15 and 60 years of age. Symptoms include weakness of the facial and tongue muscles, hand tremor, muscle cramps, dysphagia, dysarthria, and excessive development of male breasts and mammary glands. Weakness usually begins in the pelvis before spreading to the limbs. Some individuals develop noninsulin-dependent diabetes mellitus.

The course of the disorder varies but is generally slowly progressive. Individuals tend to remain ambulatory until late in the disease. The life expectancy for individuals with Kennedy disease is usually normal.

Congenital SMA with arthrogryposis (persistent contracture of joints with fixed abnormal posture of the limb) is a rare disorder. Manifestations include severe contractures, scoliosis, chest deformity, respiratory problems, unusually small jaws, and drooping of the upper eyelids.

Progressive bulbar palsy, also called progressive bulbar atrophy, involves the bulb-shaped brain stem—the region that controls lower motor neurons needed for swallowing, speaking, chewing, and other functions. Symptoms include pharyngeal muscle weakness (involved with swallowing), weak jaw and facial muscles, progressive loss of speech, and tongue muscle atrophy. Limb weakness with both lower and upper motor neuron signs is almost always evident but less prominent. Affected persons have outbursts of laughing or crying (called emotional lability). Individuals eventually become unable to eat or speak and are at increased risk of choking and aspiration pneumonia, which is caused by the passage of liquids and food through the vocal folds and into the lower airways and lungs. Stroke and myasthenia gravis each have certain symptoms that are similar to those of progressive bulbar palsy and must be ruled out prior to diagnosing this disorder. In about 25 percent of ALS cases early symptoms begin with bulbar involvement. Some 75 percent of individuals with classic ALS eventually show some bulbar involvement. Many clinicians believe that progressive bulbar palsy by itself, without evidence of abnormalities in the arms or legs, is extremely rare.

Pseudobulbar palsy, which shares many symptoms of progressive bulbar palsy, is characterized by upper motor neuron degeneration and progressive loss of the ability to speak, chew, and swallow. Progressive weakness in facial muscles leads to an expressionless face. Individuals may develop a gravelly voice and an increased gag reflex. The tongue may become immobile and unable to protrude from the mouth. Individuals may also experience emotional lability.

Primary lateral sclerosis (PLS) affects only upper motor neurons and is nearly twice as common in men as in women. Onset generally occurs after age 50. The cause of PLS is unknown. It occurs when specific nerve cells in the cerebral cortex (the thin layer of cells covering the brain which is responsible for most higher level mental functions) that control voluntary movement gradually degenerate, causing the muscles under their control to weaken. The syndrome—which scientists believe is only rarely hereditary—progresses gradually over years or decades, leading to stiffness and clumsiness of the affected muscles. The disorder usually affects the legs first, followed by the body trunk, arms and hands, and, finally, the bulbar muscles. Symptoms may include difficulty with balance, weakness and stiffness in the legs, clumsiness, spasticity in the legs which produces slowness and stiffness of movement, dragging of the feet (leading to an inability to walk), and facial involvement resulting in dysarthria (poorly articulated speech). Major differences between ALS and PLS (considered a variant of ALS) are the motor neurons involved and the rate of disease progression. PLS may be mistaken for spastic paraplegia, a hereditary disorder of the upper motor neurons that causes spasticity in the legs and usually starts in adolescence. Most neurologists follow the affected individual's clinical course for at least 3 years before making a diagnosis of PLS. The disorder is not fatal but may affect quality of life. PLS often develops into ALS.

Progressive muscular atrophy (PMA) is marked by slow but progressive degeneration of only the lower motor neurons. It largely affects men, with onset earlier than in other MNDs. Weakness is typically seen first in the hands and then spreads into the lower body, where it can be severe. Other symptoms may include muscle wasting, clumsy hand movements, fasciculations, and muscle cramps. The trunk muscles and respiration may become affected. Exposure to cold can worsen symptoms. The disease develops into ALS in many instances.

Post-polio syndrome (PPS) is a condition that can strike polio survivors decades after their recovery from poliomyelitis. PPS is believed to occur when injury, illness (such as degenerative joint disease), weight gain, or the aging process damages or kills spinal cord motor neurons that remained functional after the initial polio attack. Many scientists believe PPS is latent weakness among muscles previously affected by poliomyelitis and not a new MND. Symptoms include fatigue, slowly progressive muscle weakness, muscle atrophy, fasciculations, cold intolerance, and muscle and joint pain. These symptoms appear most often among muscle groups affected by the initial disease. Other symptoms include skeletal deformities such as scoliosis and difficulty breathing, swallowing, or sleeping. Symptoms are more frequent among older people and those individuals most severely affected by the earlier disease. Some individuals experience only minor symptoms, while others develop SMA and, rarely, what appears to be, but is not, a form of ALS. PPS is not usually life threatening. Doctors estimate the incidence of PPS at about 25 to 50 percent of survivors of paralytic poliomyelitis.

Neuronal TDP-43 proteinopathies contemplated herein may also be associated with diseases other than ALS, such as frontotemporal dementia (FTD), AD, Perry syndrome, chronic traumatic encephalopathy, ALS/Parkinsonism-dementia complex of Guam, hippocampal sclerosis and multisystem proteinopathy. A non-exclusive list of relevant TDP-43 proteinopathies includes Alzheimer's disease (AD), frontotemporal lobar degeneration, corticobasal degeneration, progressive supranuclear palsy, Gerstmann Straussler Scheinker, neurodegeneration with brain iron accumulation, globular glial tauopathies, primary age-related tauopathy, age-related tau astrogliopathy, post-encephalitic parkinsonism, subacute sclerosis panencephalitis, pantothenate kinase-associated neurodegeneration, chronic traumatic encephalopathy, Down syndrome, early-onset AD, myotonic dystrophy, lipofuscinosis, Niemann-Pick disease, type C, Alexander disease, Perry syndrome, Cockayne syndrome, ganglioglioma/gangliocytoma, pilocytic astrocytoma, lead encephalopathy, traumatic brain injury (acute) and Inclusion body myositis, as presented for example in Chornenkyy et al. (Laboratory Investigation 99:993-1007 (2019)).

4.4 Alzheimer's Disease

The main hallmarks of AD are: (1) progressive accumulation of beta-amyloid (A13 peptide in so called neuritic plaques) outside neurons, interfering with the neuron-to-neuron communication at synapses and possibly contributing to cell death; (2) AR peptides also accumulate as so-called vascular amyloid around the blood vessels of the brain, thereby interfering with the uptake of essential nutrients from the blood into the brain; (3) abnormal deposits of the protein tau (neurofibrillary tangles) inside neurons, blocking the transport of cargo inside neurons. This is a major driver of neuronal dysfunction and cell death. Eventually, both amyloid deposits and tangles cause irreversible damage in the brain, leading to atrophy of the brain and a loss of cognitive function. The most common early symptom of AD is difficulty in remembering recent events and as the disease advances, symptoms can include problems with language, disorientation (including easily getting lost), mood swings, loss of motivation, not managing self-care, and behavioral issues. As a person's condition declines, they often withdraw from family and society. Gradually, bodily functions are lost, ultimately leading to death. Although the speed of progression can vary, the typical life expectancy following diagnosis is three to nine years.

In some embodiments, the methods described herein further comprise selecting a subject diagnosed with a neurodegenerative condition, suitably one associated with a neuronal TDP-43 proteinopathy. A subject suffering from a neurodegenerative condition can be selected based on the symptoms presented. For example, a subject suffering from ALS may show symptoms of fasciculations, cramps, tight and stiff muscles (spasticity), twitching in arms, shoulder or tongue, muscle weakness affecting a hand, arm or leg, slurred and nasal speech, or difficulty chewing or swallowing.

In some embodiments, the methods described herein further comprise selecting a subject at risk of developing a neurodegenerative condition, suitably one associated with a neuronal TDP-43 proteinopathy. A subject at risk of developing a neurodegenerative condition can be selected based on a genetic diagnostic test (e.g., for a mutation in a gene associated with a neurodegenerative condition or based on the symptoms presented).

### 5. Methods of Treatment

Certain aspects of the present disclosure relate to methods for treating neurodegenerative conditions, particularly ones that are associated with a neuronal TDP-43 proteinopathy, and/or conditions characterized by neuronal degeneration. Accordingly, an aspect of the present disclosure relates to a method of treating or inhibiting the development of a neurodegenerative condition that is suitably associated with a neuronal TDP-43 proteinopathy in a subject in need thereof, wherein the method comprises administering to the subject an effective amount of an agent that enhances or increases the level or activity of cyclin F in a neuron (e.g., a motor neuron) of the subject. In another aspect, the present disclosure relates to a method of treating or inhibiting the development of a condition characterized by neuron degeneration and TDP-43 proteinopathy in a subject in need thereof, wherein the method comprises administering to the subject an effective amount of an agent that enhances or increases the level or activity of cyclin F in a neuron (e.g., a motor neuron) of the subject.

Suitably, the agent enhances or increases the level or activity of cyclin F and enhances neuronal survival (e.g., a motor neuron survival) and/or inhibits neuronal degeneration (e.g., a motor neuron degeneration) in the subject. In some embodiments, the agent enhances or increases the level or activity of cyclin F and ameliorates at least one symptom associated with the neurodegenerative condition in the subject. In some embodiments, the agent enhances or increases the level or activity of cyclin F and treats the subject's neurodegenerative condition. In some embodiments, the agent enhances or increases the level or activity of cyclin F and prevents the subject from developing a neurodegenerative condition. In some embodiments, the agent enhances or increases the level or activity of cyclin F and prevents the subject's neurodegenerative condition from progressing.

In some embodiments, the agent increases the level of a SCF^{Cyclin F} complex comprising a substrate of the complex (e.g., TDP-43) and enhances neuron survival (e.g., a motor neuron survival) and/or inhibits neuronal degeneration (e.g., a motor neuron degeneration) in the subject. In some embodiments, the agent increases the level of a SCF^{Cyclin F} complex comprising a substrate of the complex (e.g., TDP-43) and ameliorates at least one symptom associated with the neurodegenerative condition in the subject. In some embodiments, the agent increases the level of a SCF^{Cyclin F} complex comprising a substrate of the complex (e.g., TDP-43) and treats the subject's neurodegenerative condition. In some embodiments, the agent increases the level of a SCF^{Cyclin F} complex comprising a substrate of the complex (e.g., TDP-43) and prevents the subject from developing a neurodegenerative condition. In some embodiments, the agent increases the level of a SCF^{Cyclin F} complex comprising a substrate of the complex (e.g., TDP-43) and prevents the subject's neurodegenerative condition from progressing.

In some embodiments, the agent decreases the amount of a protein that is susceptible to protein aggregation (e.g., TDP-43) and enhances neuronal survival (e.g., a motor neuron survival) and/or inhibits neuronal degeneration (e.g., a motor neuron degeneration) in the subject. In some embodiments, the agent decreases the amount of a protein that is susceptible to protein aggregation (e.g., TDP-43) and ameliorates at least one symptom associated with the neurodegenerative condition in the subject. In some embodiments, the agent decreases the amount of a protein that is susceptible to protein aggregation (e.g., TDP-43) and treats the subject's neurodegenerative condition. In some embodiments, the agent decreases the amount of a protein that is susceptible to protein aggregation (e.g., TDP-43) and prevents the subject from developing a neurodegenerative condition. In some embodiments, the agent decreases the amount of a protein that is susceptible to protein aggregation (e.g., TDP-43) and prevents the subject's neurodegenerative condition from progressing.

Any agent that increases level or activity of cyclin F in a neuron (e.g., a motor neuron) can be used in the embodiments described herein.

In some embodiments, the subject is a human.

In some embodiments, the subject selected for treatment of a neurodegenerative condition, or a condition characterized by motor neuron degeneration. In some embodiments, the subject is at risk of developing a neurodegenerative condition, particularly one that is associated with a neuronal TDP-43 proteinopathy, or a condition characterized by motor neuron degeneration. In some embodiments, the subject is suspected of having a neurodegenerative condition, particularly one that is associated with a neuronal TDP-43 proteinopathy, or a condition characterized by motor neuron degeneration. In some embodiments, the subject is suffering from a neurodegenerative condition, particularly one that is associated with a neuronal TDP-43 proteinopathy. The neurodegenerative condition can be any neurodegenerative condition described herein. In some embodiments, the neurodegenerative condition is marked by motor neuron degeneration. In some embodiments, the neurodegenerative condition is a motor neuron disease. In some embodiments, the neurodegenerative condition is ALS. In some embodiments, the neurodegenerative condition is FTD. In some embodiments, the neurodegenerative condition includes neuronal degeneration other than motor neuron degeneration. some embodiments, the neurodegenerative condition is AD.

In some embodiments, another therapeutic agent is also administered to the subject. Such another therapeutic or “ancillary” agent is typically administered concurrently with the cyclin F-enhancing agent. For example, the therapeutic agent can be administered in the same formulation or in separate formulations Ex e.g., Butyrates, Valproic acid, Hydroxyurea or Riluzole. In some embodiments, the agents described herein are used in combination with another therapeutic agent suitable for use in treating one or more symptoms of ALS, including, but not limited to, one or more of (i) hydrogenated pyrido [4,3-b] indoles or pharmaceutically acceptable salts thereof and (ii) agents that promote or increase the supply of energy to muscle cells, COX-2 inhibitors, poly(ADP-ribose)polymerase-1 (PARP-I) inhibitors, 3OS ribosomal protein inhibitors, NMDA antagonists, NMDA receptor antagonists, sodium channel blockers, glutamate release inhibitors, K(V)4.3 channel blockers, anti-inflammatory agents, 5-HT1A receptor agonists, neurotrophic factor enhancers, agents that promote motoneuron phenotypic survival and/or neuritogenesis, agents that protect the blood brain barrier from disruption, inhibitors of the production or activity of one or more proinflammatory cytokines, immunomodulators, neuroprotectants, modulators of the function of astrocytes, antioxidants (such as small molecule catalytic antioxidants), free radical scavengers, agents that decrease the amount of one or more reactive oxygen species, agents that inhibit the decrease of non-protein thiol content, stimulators of a normal cellular protein repair pathway (such as agents that activate molecular chaperones), neurotrophic agents, inhibitors of nerve cell death, stimulators of neurite growth, agents that prevent the death of nerve cells and/or promote regeneration of damaged brain tissue, cytokine modulators, agents that reduce the level of activation of microglial cells, cannabinoid CB1 receptor ligands, nonsteroidal anti-inflammatory drugs, cannabinoid CB2 receptor ligands, creatine, creatine derivatives, stereoisomers of a dopamine receptor agonist such as pramipexole hydrochloride, ciliary neurotrophic factors, agents that encode a ciliary neurotrophic factor, glial derived neurotrophic factors, agents that encode a glial derived neurotrophic factor, neurotrophin 3, agents that encode neurotrophin 3, or any combination thereof.

In some embodiments, the agents described herein are used in combination with another therapeutic agent suitable for use in treating one or more symptoms of ALS or FTD, including, but not limited to, one or more of antibiotics (e.g., Aminoglycosides, Cephalosporins, Chloramphenicol, Clindamycin, Erythromycins, Fluoroquinolones, Macrolides, Azolides, Metronidazole, Penicillins, Tetracyclines, Trimethoprim-sulfamethoxazole, Vancomycin), steroids (e.g., Andranes (e.g., Testosterone), Cholestanes (e.g., Cholesterol), Cholic acids (e.g., Cholic acid), Corticosteroids (e.g., Dexamethasone), Estraenes (e.g., Estradiol), Pregnanes (e.g., Progesterone), narcotic and non-narcotic analgesics (e.g., Morphine, Codeine, Heroin, Hydromorphone, Levorphanol, Meperidine, Methadone, Oxydone, Propoxyphene, Fentanyl, Methadone, Naloxone, Buprenorphine, Butorphanol, Nalbuphine, Pentazocine), anti-inflammatory agents (e.g., Alclofenac, Alclometasone Dipropionate, Algestone Acetonide, alpha Amylase, Amcinafal, Amcinafide, Amfenac Sodium, Amiprilose Hydrochloride, Anakinra, Anirolac, Anitrazafen, Apazone, Balsalazide Disodium, Bendazac, Benoxaprofen, Benzydamine Hydrochloride, Bromelains, Broperamole, Budesonide, Carprofen, Cicloprofen, Cintazone, Cliprofen, Clobetasol Propionate, Clobetasone Butyrate, Clopirac, Cloticasone Propionate, Cormethasone Acetate, Cortodoxone, Decanoate, Deflazacort, Delatestryl, Depo-Testosterone, Desonide, Desoximetasone, Dexamethasone Dipropionate, Diclofenac Potassium, Diclofenac Sodium, Diflorasone Diacetate; Diflumidone Sodium, Diflunisal, Difluprednate, Diftalone, Dimethyl Sulfoxide, Drocinonide, Endrysone, Enlimomab, Enolicam Sodium, Epirizole, Etodolac, Etofenamate, Felbinac, Fenamole, Fenbufen, Fenclofenac, Fenclorac, Fendosal, Fenpipalone, Fentiazac, Flazalone, Fluazacort, Flufenamic Acid, Flumizole, Flunisolide Acetate, Flunixin, Flunixin Meglumine, Fluocortin Butyl, Fluorometholone Acetate, Fluquazone, Flurbiprofen, Fluretofen, Fluticasone Propionate, Furaprofen, Furobufen, Halcinonide, Halobetasol Propionate, Halopredone Acetate, Ibufenac, Ibuprofen, Ibuprofen Aluminum, Ibuprofen Piconol, Ilonidap, Indomethacin, Indomethacin Sodium, Indoprofen, Indoxole, Intrazole, Isoflupredone Acetate, Isoxepac, Isoxicam, Ketoprofen, Lofemizole Hydrochloride, Lomoxicam, Loteprednol Etabonate, Meclofenamate Sodium, Meclofenamic Acid, Meclorisone Dibutyrate, Mefenamic Acid, Mesalamine, Meseclazone, Mesterolone, Methandrostenolone, Methenolone, Methenolone Acetate, Methylprednisolone Suleptanate, Morniflumate, Nabumetone, Nandrolone, Naproxen, Naproxen Sodium, Naproxol, Nimazone, Olsalazine Sodium, Orgotein, Orpanoxin, Oxandrolane, Oxaprozin, Oxyphenbutazone, Oxymetholone, Paranyline Hydrochloride, Pentosan Polysulfate Sodium, Phenbutazone Sodium Glycerate, Pirfenidone, Piroxicam, Piroxicam Cinnamate, Piroxicam Olamine, Pirprofen, Prednazate, Prifelone, Prodolie Acid, Proquazone, Proxazole, Proxazole Citrate, Rimexolone, Romazarit, Salcolex, Salnacedin, Salsalate, Sanguinarium Chloride, Seclazone, Sermetacin, Stanozolol, Sudoxicam, Sulindac, Suprofen, Talmetacin, Talniflumate, Talosalate, Tebufelone, Tenidap, Tenidap Sodium, Tenoxicam, Tesicam, Tesimide, Testosterone, Testosterone Blends, Tetrydamine, Tiopinac, Tixocortol Pivalate, Tolmetin, Tolmetin Sodium, Triclonide, Triflumidate, Zidometacin, Zomepirac Sodium), or anti-histaminic agents (e.g., Ethanolamines (like diphenhydrmine carbinoxa mine), Ethylenediamine (like tripelennamine pyrilamine), Alkylamine (like chlorpheniramine, dexchlorpheniramine, brompheniramine, triprolidine), other anti-histamines like astemizole, loratadine, fexofenadine, Bropheniramine, Clemastine, Acetaminophen, Pseudoephedrine, Triprolidine).

In some embodiments, the agents described herein are used in combination with another therapeutic agent suitable for use in treating one or more symptoms of AD, including, but not limited to, cognition-enhancing agents such as but not limited to donepezil, rivastigmine, memantine and galantamine

### 6. Formulations and Administration

For administration to a subject, the agents described herein can be administered orally, parenterally, for example, subcutaneously, intravenously, intramuscularly, intraperitoneally, by intranasal instillation, or by application to mucous membranes, such as, that of the nose, throat, and bronchial tubes. One method for targeting the nervous system, such as spinal cord glia, is by intrathecal delivery. The targeted agent is released into the surrounding CSF and/or tissues and the released compound can penetrate into the spinal cord parenchyma, just after acute intrathecal injections. For a comprehensive review on drug delivery strategies including CNS delivery, see Ho et al., Curr. Opin. Mol. Ther. (1999), 1:336-3443; Groothuis et al., J. Neuro Virol. (1997), 3:387-400; and Jan, Drug Delivery Systmes: Technologies and Commercial Opportunities, Decision Resources, 1998, content of all which is incorporate herein by reference.

They can be administered alone or with suitable pharmaceutical carriers, and can be in solid or liquid form such as, tablets, capsules, powders, solutions, suspensions, or emulsions.

The agents can be formulated in pharmaceutically acceptable compositions which comprise an effective amount of the agent, formulated together with one or more pharmaceutically acceptable carriers (additives) and/or diluents. The agents can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds and/or agents can be implanted into a patient or injected using a drug delivery system. See, for example, Urquhart, et al. (1984. Ann. Rev. Pharmacol. Toxicol. 24: 199-236); Lewis, ed. “Controlled Release of Pesticides and Pharmaceuticals” (Plenum Press, New York, 1981); U.S. Pat. No. 3,773,919; and U.S. Pat. No. 353,270,960.

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

Pharmaceutically-acceptable antioxidants include, but are not limited to, (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lectithin, propyl gallate, alpha-tocopherol, and the like; and (3) metal chelating agents, such as citric acid, ethylenediamine tetraacetic acid (EDTA), sorbitol, tartaric acid, phosphoric acids, and the like.

PEG includes within it scope any ethylene glycol polymer that contains about 20 to about 2000000 linked monomers, typically about 50-1000 linked monomers, usually about 100-300. Polyethylene glycols include PEGs containing various numbers of linked monomers, e.g., PEG20, PEG30, PEG40, PEG60, PEG80, PEG100, PEG115, PEG200, PEG 300, PEG400, PEG500, PEG600, PEG1000, PEG1500, PEG2000, PEG3350, PEG4000, PEG4600, PEG5000, PEG6000, PEG8000, PEG11000, PEG12000, PEG2000000 and any mixtures thereof.

The agents can be formulated in a gelatin capsule, in tablet form, dragee, syrup, suspension, topical cream, suppository, injectable solution, or kits for the preparation of syrups, suspension, topical cream, suppository or injectable solution just prior to use. Also, compounds and/or agents can be included in composites, which facilitate its slow release into the blood stream, e.g., silicon disc, polymer beads.

The formulations can conveniently be presented in unit dosage form and may be prepared by any of the methods well known in the art of pharmacy. Techniques, excipients and formulations generally are found in, e.g., Remington's Pharmaceutical Sciences, Mack Publishing Co., Easton, Pa. 1985, 17th edition, Nema et al., PDA J. Pharm. Sci. Tech. 1997 51:166-171. Methods to make invention formulations include the step of bringing into association or contacting an active agent with one or more excipients or carriers. In general, the formulations are prepared by uniformly and intimately bringing into association one or more agents with liquid excipients or finely divided solid excipients or both, and then, if appropriate, shaping the product.

The preparative procedure may include the sterilization of the pharmaceutical preparations. The agents may be mixed with auxiliary agents such as lubricants, preservatives, stabilizers, salts for influencing osmotic pressure, etc., which do not react deleteriously with the agents.

Examples of injectable forms include solutions, suspensions and emulsions. Injectable forms also include sterile powders for extemporaneous preparation of injectable solutions, suspensions or emulsions. The agents of the present invention can be injected in association with a pharmaceutical carrier such as normal saline, physiological saline, bacteriostatic water, Cremophor™ EL (BASF, Parsippany, N.J.), phosphate buffered saline (PBS), Ringer's solution, dextrose solution, ethanol, polyol (e.g., glycerol, propylene glycol, and liquid polyethylene glycol), vegetable oils, and suitable mixtures thereof, and other aqueous carriers known in the art. Appropriate non-aqueous carriers may also be used and examples include fixed oils and ethyl oleate. In all cases, the composition must be sterile and should be fluid to the extent that easy syringability exists. It must be stable under the conditions of manufacture and storage and must be preserved against the contaminating action of microorganisms such as bacteria and fungi. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin. A suitable carrier is 5% dextrose in saline. Frequently, it is desirable to include additives in the carrier such as buffers and preservatives or other substances to enhance isotonicity and chemical stability.

In some embodiments, agents described herein can be administrated encapsulated within liposomes. The manufacture of such liposomes and insertion of molecules into such liposomes being well known in the art, for example, as described in U.S. Pat. No. 4,522,811. Liposomal suspensions (including liposomes targeted to particular cells, e.g., a pituitary cell) can also be used as pharmaceutically acceptable carriers.

In one embodiment, the agents are prepared with carriers that will protect the compound and/or agent against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc.

In the case of oral ingestion, excipients useful for solid preparations for oral administration are those generally used in the art, and the useful examples are excipients such as lactose, sucrose, sodium chloride, starches, calcium carbonate, kaolin, crystalline cellulose, methyl cellulose, glycerin, sodium alginate, gum arabic and the like, binders such as polyvinyl alcohol, polyvinyl ether, polyvinyl pyrrolidone, ethyl cellulose, gum arabic, shellac, sucrose, water, ethanol, propanol, carboxymethyl cellulose, potassium phosphate and the like, lubricants such as magnesium stearate, talc and the like, and further include additives such as usual known coloring agents, disintegrators such as alginic acid and PRIMOGEL™, and the like.

The agents can be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these compounds and/or agents may be incorporated with excipients and used in the form of tablets, capsules, elixirs, suspensions, syrups, and the like. Such compositions and preparations should contain at least 0.1% of compound and/or agent. The percentage of the agent in these compositions may, of course, be varied and may conveniently be between about 2% to about 60% of the weight of the unit. The amount of compound and/or agent in such therapeutically useful compositions is such that a suitable dosage will be obtained. Preferred compositions according to the present invention are prepared so that an oral dosage unit contains between about 100 and 2000 mg of compound and/or agent.

Examples of bases useful for the formulation of suppositories are oleaginous bases such as cacao butter, polyethylene glycol, lanolin, fatty acid triglycerides, witepsol (trademark, Dynamite Nobel Co. Ltd.) and the like. Liquid preparations may be in the form of aqueous or oleaginous suspension, solution, syrup, elixir and the like, which can be prepared by a conventional way using additives.

The compositions can be given as a bolus dose, to maximize the circulating levels for the greatest length of time after the dose. Continuous infusion may also be used after the bolus dose.

The agents can also be administrated directly to the airways in the form of an aerosol. For administration by inhalation, the agents in solution or suspension can be delivered in the form of an aerosol spray from pressured container or dispenser which contains a suitable propellant, e.g., a gas such as carbon dioxide, or hydrocarbon propellant like propane, butane or isobutene. The agents can also be administrated in a no-pressurized form such as in an atomizer or nebulizer.

The agents can also be administered parenterally. Solutions or suspensions of these agents can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

It may be advantageous to formulate oral or parenteral compositions in dosage unit form for ease of administration and uniformity of dosage.

Administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the agents are formulated into ointments, salves, gels, or creams as generally known in the art.

The agents can be administrated to a subject in combination with other pharmaceutically active agents. Exemplary pharmaceutically active compounds and/or agents include, but are not limited to, those found in Harrison's Principles of Internal Medicine, 13.sup.th Edition, Eds. T. R. Harrison et al. McGraw-Hill N.Y., NY; Physician's Desk Reference, 50.sup.th Edition, 1997, Oradell New Jersey, Medical Economics Co.; Pharmacological Basis of Therapeutics, 8.sup.th Edition, Goodman and Gilman, 1990; United States Pharmacopeia, The National Formulary, USP XII NF XVII, 1990, the complete contents of all of which are incorporated herein by reference. In some embodiments, the pharmaceutically active agent is selected from the group consisting of butyrates, valproic acid, hydroxyurea and Riluzole.

The agents and the other pharmaceutically active agent can be administrated to the subject in the same pharmaceutical composition or in different pharmaceutical compositions (at the same time or at different times). For example, an Aurora kinase inhibitor and an additional agent for treating a neurodegenerative condition can be administrated to the subject in the same pharmaceutical composition or in different pharmaceutical compositions (at the same time or at different times).

The amount of agent which can be combined with a carrier material to produce a single dosage form will generally be that amount of the agent which produces a therapeutic effect. Generally, out of one hundred percent, this amount will range from about 0.1% to 99% of compound, preferably from about 5% to about 70%, most preferably from 10% to about 30%.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both. A syrup may contain, in addition to the active ingredient, sucrose as a sweetening agent, methyl and propylparabens as preservatives, a dye, and flavoring such as cherry or orange flavor.

The pharmaceutical compositions can be included in a container, pack, or dispenser together with instructions for administration.

Guidance regarding the efficacy and dosage which will deliver an effective amount of a compound and/or agent to treat ALS or FTD can be obtained from animal models of ALS or FTD, see e.g., those described in Hsieh-Li et al. (2000. Nature Genetics 24:66-70) and references cited therein.

Toxicity and therapeutic efficacy can be determined by standard pharmaceutical procedures in cell cultures or experimental animals, e.g., for determining the LD50 (the dose lethal to 50% of the population) and the ED50 (the dose effective in 50% of the population). The dose ratio between toxic and therapeutic effects is the therapeutic index and it can be expressed as the ratio LD50/ED50. Compositions that exhibit large therapeutic indices, are preferred.

The data obtained from the cell culture assays and animal studies can be used in formulating a range of dosage for use in humans. The dosage of such compounds and/or agents lies preferably within a range of circulating concentrations that include the ED50 with little or no toxicity. The dosage may vary within this range depending upon the dosage form employed and the route of administration utilized.

The effective dose can be estimated initially from cell culture assays. A dose may be formulated in animal models to achieve a circulating plasma concentration range that includes the IC50 (i.e., the concentration of the therapeutic which achieves a half-maximal inhibition of symptoms) as determined in cell culture. Levels in plasma may be measured, for example, by high performance liquid chromatography. The effects of any particular dosage can be monitored by a suitable bioassay. Examples of suitable bioassays include DNA replication assays, transcription based assays, GDF-8 binding assays, and immunological assays.

The dosage may be determined by a physician and adjusted, as necessary, to suit observed effects of the treatment. Generally, the compositions are administered so that the compound and/or agent is given at a dose from 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. For antibody compounds and/or agents, one preferred dosage is 0.1 mg/kg of body weight (generally 10 mg/kg to 20 mg/kg). If the antibody is to act in the brain, a dosage of 50 mg/kg to 100 mg/kg is usually appropriate.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the polypeptides. The desired dos can be administered at one time or divided into subdoses, e.g., 2-4 subdoses and administered over a period of time, e.g., at appropriate intervals through the day or other appropriate schedule. Such sub-doses can be administered as unit dosage forms. Examples of dosing schedules are administration once a week, twice a week, three times a week, daily, twice daily, three times daily or four or more times daily.

### 7. Kits

An agent described herein can be provided in a kit. The kit includes (a) the agent, e.g., a composition that includes the agent, and (b) informational material. The informational material can be descriptive, instructional, marketing or other material that relates to the methods described herein and/or the use of the agent for the methods described herein. For example, the informational material describes methods for administering the agent to enhance motor neuron survival, treat or inhibit the development of a neurodegenerative condition, particularly one that is associated with neuronal TDP-43 proteinopathy (e.g., ALS, FTD, AD, etc.), or at least one symptom of the neurodegenerative condition, or a condition associated with dysfunctional or decreases neurons (e.g., motor neurons).

In one embodiment, the informational material can include instructions to administer the agent in a suitable manner, e.g., in a suitable dose, dosage form, or mode of administration (e.g., a dose, dosage form, or mode of administration described herein). In another embodiment, the informational material can include instructions for identifying a suitable subject, e.g., a human, e.g., an adult human. The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is a link or contact information, e.g., a physical address, email address, hyperlink, website, or telephone number, where a user of the kit can obtain substantive information about the modulator and/or its use in the methods described herein. Of course, the informational material can also be provided in any combination of formats.

In addition to the agent, the composition of the kit can include other ingredients, such as a solvent or buffer, a stabilizer or a preservative, and/or a second agent for treating a condition or disorder described herein. Alternatively, the other ingredients can be included in the kit, but in different compositions or containers than the agent. In such embodiments, the kit can include instructions for admixing the agent and the other ingredients, or for using the agent together with the other ingredients.

The agent can be provided in any form, e.g., liquid, dried or lyophilized form. It is preferred that the agent be substantially pure and/or sterile. When the agent is provided in a liquid solution, the liquid solution preferably is an aqueous solution, with a sterile aqueous solution being preferred. When the compound and/or agent is provided as a dried form, reconstitution generally is by the addition of a suitable solvent. The solvent, e.g., sterile water or buffer, can optionally be provided in the kit.

The kit can include one or more containers for the composition containing the compound and/or agent. In some embodiments, the kit contains separate containers, dividers or compartments for the agent (e.g., in a composition) and informational material. For example, the agent (e.g., in a composition) can be contained in a bottle, vial, or syringe, and the informational material can be contained in a plastic sleeve or packet. In other embodiments, the separate elements of the kit are contained within a single, undivided container. For example, the agent (e.g., in a composition) is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agent (e.g., in a composition). For example, the kit includes a plurality of syringes, ampules, foil packets, or blister packs, each containing a single unit dose of the agent. The containers of the kits can be air tight and/or waterproof.

The agent (e.g., in a composition) can be administered to a subject, e.g., an adult subject, e.g., a subject in need of enhancing survival or viability of neurons (e.g., motor neurons), and/or inhibiting degeneration of a neuron (e.g., a motor neuron) and/or inhibiting abnormal protein accumulation in a neuron (e.g., a motor neuron). The method can include evaluating a subject, e.g., to evaluate the presence of neuronal TDP-43 proteinopathy in the subject, thereby identifying that the subject may be susceptible to treatment with the cyclin F-enhancing agents described herein.

In order that the invention may be readily understood and put into practical effect, particular preferred embodiments will now be described by way of the following non-limiting examples.

### Experimental

### Cyclin F is an Interaction Partner of TDP-43

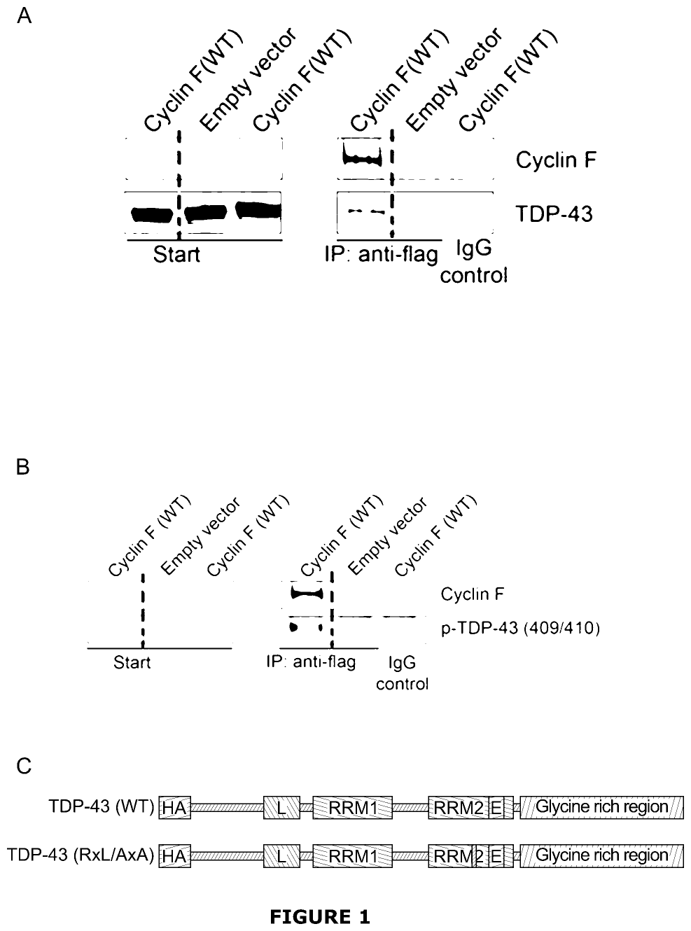

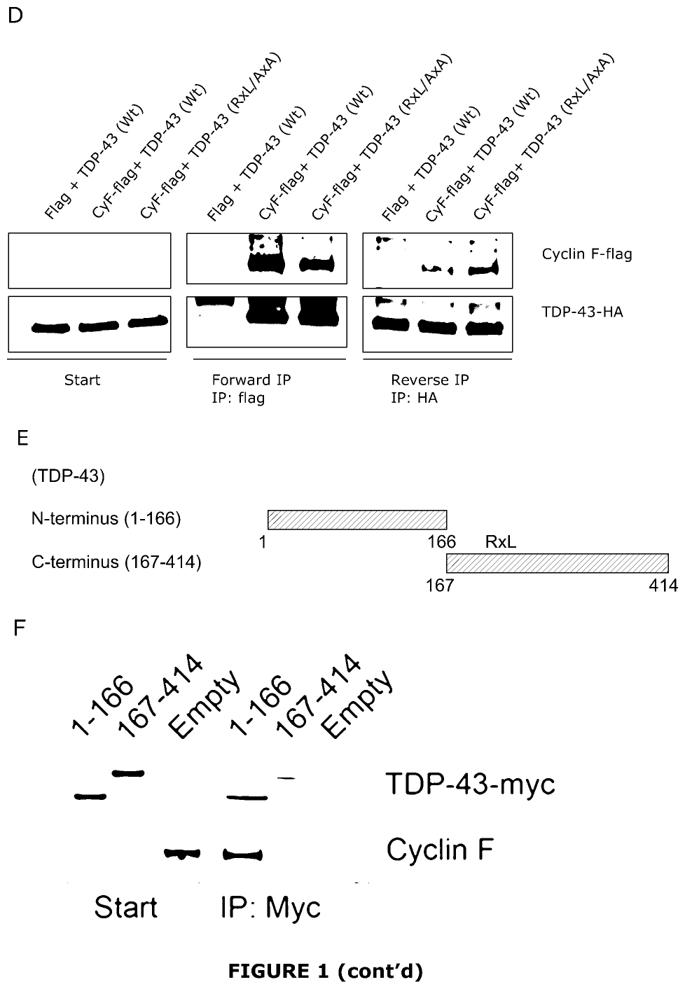

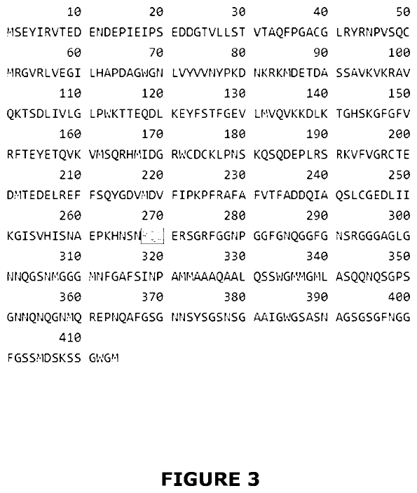

Given that cyclin F overexpression leads to the hyperubiquitylation of TDP-43 in neuron-like cells (Williams, Topp et al. 2016), we questioned whether cyclin F could be directly responsible for mediating ubiquitylation of TDP-43. Accordingly, we first assessed whether cyclin F could bind TDP-43. Cyclin F is known to bind substrates through a Cy motif (RxL) present on substrates (Dankert, Rona et al. 2016). Analysis of the amino acid sequence of TDP-43 revealed a single RxL motif between amino acids 268-270 (FIG. 3). In order to determine if cyclin F binds TDP-43, we conducted a series of standard immunoprecipitation experiments. Here, HEK293 cells were transfected with constructs encoding Flag-tagged cyclin F and HA-tagged TDP-43. At 24 hours post-transfection, cells were lysed and Flag-cyclin F was enriched by immunoprecipitation. Immunoblot analysis of the eluates revealed that cyclin F co-immunoprecipitates with TDP-43 (FIG. 1A). The interaction between cyclin F and a phosphorylated form of TDP-43 was also investigated given that phosphorylated TDP-43 (409/410) is specifically associated with pathological TDP-43 inclusions. Accordingly, lysates immunoprecipitated with cyclin F-flag were analyzed by immunoblotting using antibodies recognizing phosphorylated TDP-43, revealing that cyclin F could also recognize full-length phosphorylated TDP-43 (409/410) (FIG. 1B).

In order to determine whether cyclin F interacts with TDP-43 through the typical RxL motif present in all known cyclin F substrates, constructs expressing wild-type TDP-43 or variant TDP-43 (RxL>AxA) were transfected into HEK293 cells (FIG. 1C). After 24 hours, the cells were lysed and Flag-cyclin F was immunoprecipitated and analyzed for TDP-43 by immunoblotting. TDP-43 co-immunoprecipitated with cyclin F in both wild-type form and when carrying the RxL>AxA mutation, suggesting that the binding region was independent of the RxL motif (FIG. 1D). In agreement with this data, we also conducted immunoprecipitations using full-length cyclin F and truncated fragments of TDP-43 tagged with myc (FIG. 1E). N- and C-terminal fragments of TDP-43 were over-expressed together with Flag-cyclin F, cells lysed and TDP-43 enriched by immunoprecipitation. Here, the N-terminal fragment (1-161) but not C-terminal fragment (167-414) of TDP-43 co-immunoprecipitated with cyclin F, further indicating that the interaction between cyclin F and TDP-43 was independent of the RxL motif in TDP-43 (FIG. 1F).

### Cyclin F Directly Binds and Mediates the Ubiquitylation of TDP-43

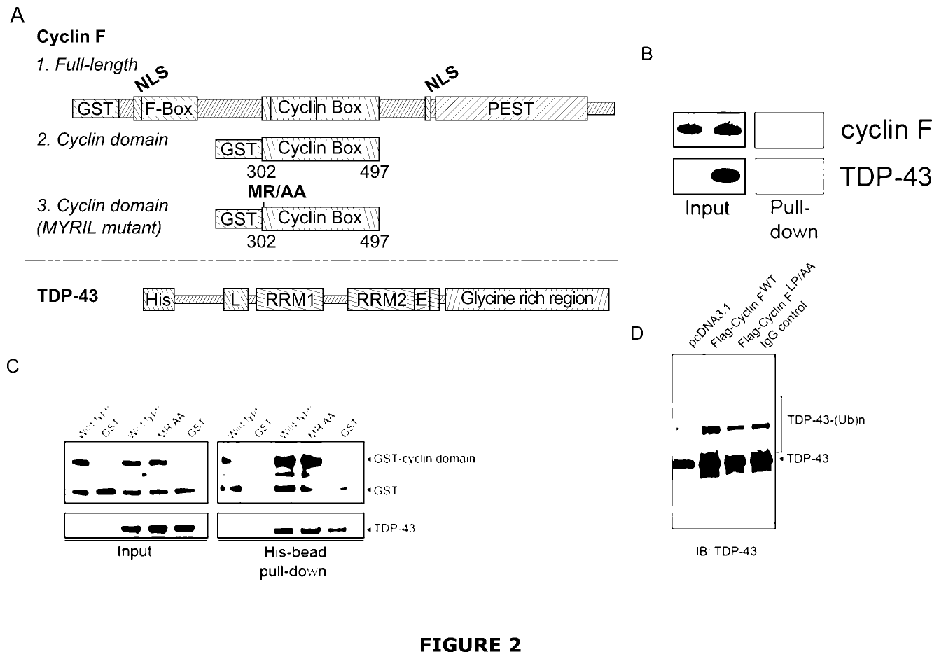

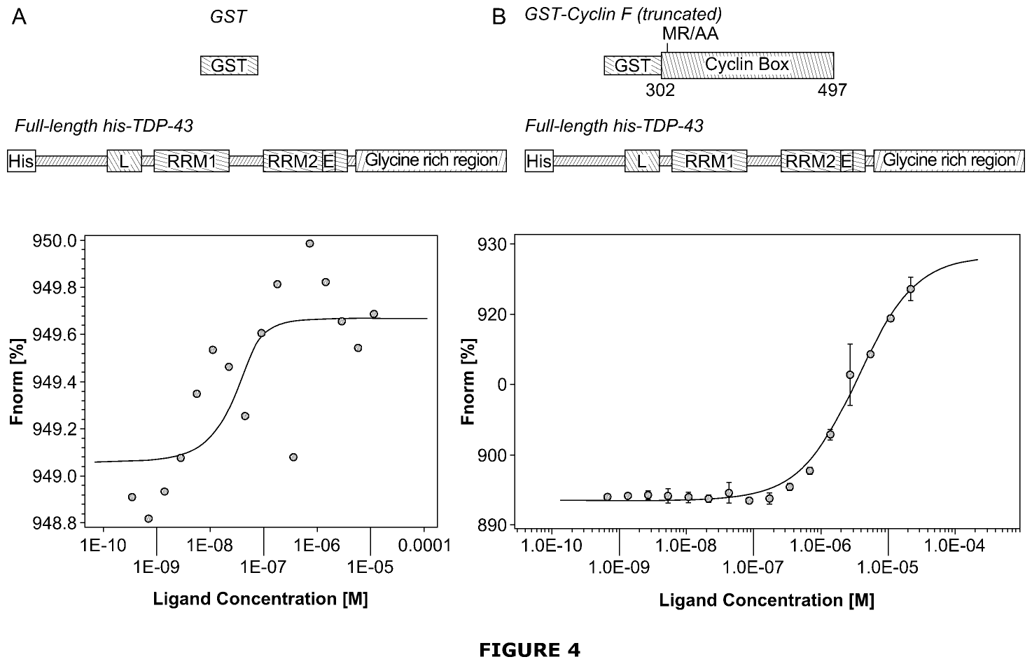

As cyclin F did not interact with TDP-43 via a typical RxL motif, the present inventors questioned whether the interaction between cyclin F and TDP-43 was indeed a direct interaction. Accordingly, they conducted a series of pull-down studies using recombinant cyclin F-GST and His-TDP-43. Here, full length TDP-43 and both full-length and truncated fragments of GST-tagged cyclin F were generated (FIG. 2A) and incubated together before Ni-NTA magnetic beads were used to isolate His-tagged TDP-43 with co-interacting protein. The results of these pull-down studies indicate that cyclin F does indeed directly interact with TDP-43 (FIG. 2B). Furthermore, the cyclin domain of cyclin F was responsible for direct binding to TDP-43 (FIG. 2C), a result validated by microscale thermophoresis (MST) (FIG. 4).

In previous studies, it has been shown that cyclin F binds substrates using a MRYIL sequence within its cyclin domain (Klein et al., 2015). This domain appears at amino acid residues 309-313 of SEQ ID NO:2 herein. Thus, an MR>AA substitution was introduced in the cyclin domain (amino acid residues 309-310 of SEQ ID NO:2) to perturb the interaction. The amino acid sequence of the MR/AA variant is set forth in SEQ ID NO:7. Consistent with the notion that cyclin F binds with TDP-43 via an atypical interaction, the MR>AA substitution did not impact the binding between recombinant cyclin F and TDP-43 (FIG. 2C).

Despite atypical binding, the present inventors evaluated whether TDP-43 is a direct ubiquitylation substrate of cyclin F (as distinct from an interacting protein). To do this, they developed an in vitro E3 ligase ubiquitylation assay, which incorporates all necessary components for protein ubiquitylation. Firstly, Flag-tagged cyclin F was transfected into HEK293 cells and immunoprecipitated cyclin F with anti-Flag antibody. The present inventors have previously demonstrated that using this methodology that cyclin F co-immunoprecipitates together with the other components of the SCF complex (Skp1, Cul 1 and Rbx1) and that this collectively retains E3 ligase activity. Immunoprecipitated cyclin F (SCF^{cyclin F}) was then used within the in vitro ubiquitylation assay using recombinant His-TDP-43 as the substrate. The results presented herein indicate that SCF^{Cyclin F} is able to mediate poly-ubiquitylation on recombinant TDP-43 in vitro (FIG. 2D). In contrast, the use of cyclin F(LP/AA), which does not form an active ubiquitin ligase, had no effect upon levels of ubiquitylated TDP-43 (FIG. 2D). Together, these data suggest that TDP-43 is a direct ubiquitylation substrate of SCF^{cyclin F}.

TDP-43 proteinopathy is seen in almost all cases of ALS and in more than half of FTD cases (Ling, Polymenidou et al., 2013). In many cases TDP-43 is found to be depleted from the nucleus as it builds up in cytoplasmic aggregates. This suggests that the loss of functional TDP-43 may be a contributing factor in ALS/FTD pathogenesis. Therefore, there is considerable interest in identifying the molecular mechanisms responsible for abnormal cytoplasmic accumulation and aggregation of TDP-43. Notably, a characteristic feature of TDP-43 present within cytoplasmic inclusions in postmortem patient tissue is hyper-ubiquitylation, which is considered to reflect disrupted proteasomal clearance of TDP-43. Accordingly, identification of endogenous pathways for ubiquitylation and clearance of TDP-43 are of considerable interest.

The present inventors now present the first report of an ALS-linked molecular pathway that causes ubiquitylation and subsequent proteasomal degradation of TDP-43. They present a series of biochemical data (immunoprecipitation, MST) that collectively demonstrate that cyclin F binds to TDP-43, and subsequently that the SCF^{cyclin F} complex ubiquitylates TDP-43. Surprisingly they found that this interaction is atypical, since it occurs independently of the R-X-L motif reported for all known cyclin F substrates, and the MRYIL substrate-recognition motif in cyclin F. It is believed that this represents the first in vivo report of a specific UPS-mediated pathway responsible for TDP-43 clearance.

### Cyclin F Overexpression in Mice Leads to Enhanced Clearance of Insoluble TDP-43 Species

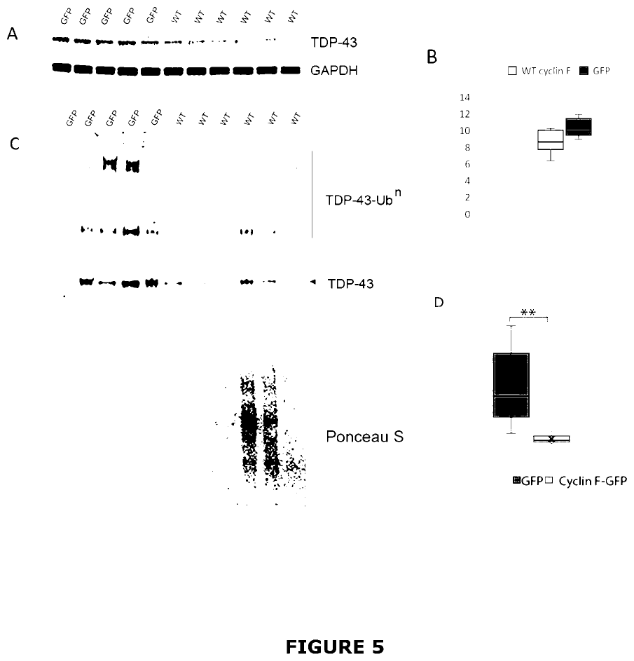

To determine the effect of cyclin F overexpression in the central nervous system of mice, AAV9-PHP.B was used to deliver expression of cyclin F or an empty vector control specifically in neurons (synapsin promotor) of wildtype mice over 8 months. Post-mortem motor cortex was obtained from these mice and the presence of RIPA-soluble and RIPA-insoluble TDP-43 were analyzed by immunoblotting. As seen in FIG. 5A, bands corresponding to TDP-43 were noted at 43 kDa as expected. In addition, higher molecular weight species were also detected, indicating the presence of ubiquitylated TDP-43 species. Densitometry analysis of immunoblots revealed that both monomeric and ubiquitylated TDP-43 was significantly reduced in mice overexpressing cyclin F, relative to the control.

### mRNA-Mediated CCNF Overexpression in TDP-43 Transgenic Zebrafish

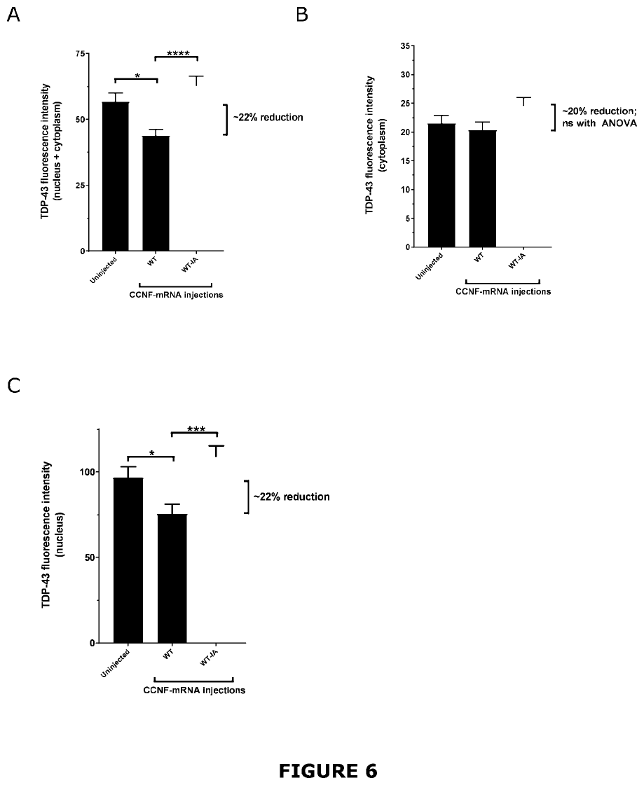

Transgenic zebrafish expressing GFP-labeled human wild type TDP-43 in motor neurons were used to assess CCNF interactions. Results presented in FIG. 6 show that injection of mRNA encoding widltype human cyclin F results in a reduction in nuclear levels of human TDP-43, whereas injection of mRNA encoding the inactive cyclin F(IP/AA) variant, which binds to TDP-43 but fails to ubiquitylate, does not result in human TDP-43 reduction.

### Materials and Methods

Plasmids and Cloning

Expression constructs encoding wild-type and S621G CCNF cDNA fused to an N-terminal tag (such as a fluorophore, or peptide-purification tag) were used as described previously (Williams et al. Nature Communications 7: 11253 (2016); Lee et al. Cell Mol Life Sci. 75(2):335-354 (2018); Hogan et al. Hum Mol Genet. 28(4):698 (2019)).

Wild-type and S621G CCNF cDNA fused to a C-terminal Flag tag was cloned into a pcDNA 3.1 vector. Gene sequences encoding GST-tagged cyclin F or the cyclin box of cyclin F were cloned into pGEX5 vectors.

Cell Culture

HEK293, Neuro-2a, SHSY5Y and NSC-34 cells used were maintained in DMEM with 10% FBS and antibiotics (100 mg/mL streptomycin and 100 U/mL penicillin). All cells were kept in a 37° C. incubator with 5% CO2 and 95% humidity. HEK293 Flp-In T-Rex cells (Thermo) were maintained under similar conditions. Cells were maintained with Zeocin until cells were transfected with Flp-recombinase, after which transfected cells were selected using Hygromycin and Blasticidin.

Flag and mCherry Affinity Purification

Either HEK293 or Neuro-2A cells were transfected with constructs encoding mCherry-cyclin F, Flag-cyclin F or TDP-43-HA using Lipofectamine 2000. Transfected cells were harvested after 24 hours and cell pellets were resuspended in NP40 lysis buffer (1% (v/v) Nonidet P-40, Tris-buffered saline (TBS), 2 mM EDTA, cOmplete protease inhibitor cocktail and phosSTOP (Roche)). Cell resuspensions were probe sonicated (10 s, Setting 3, Branson Sonifier 450) to disrupt protein aggregates. Resulting lysates were centrifuged at 14,000×g for 30 mins to remove cell debris. A 500 μg aliquot of each supernatant was incubated with either 2 μg of anti-Flag M2 (Sigma), 1 μg of anti-mCherry (Clonetech) or 1 μg of anti-TDP-43 (Abnova) for 1 hour at 4° C. To capture the antibody-protein complex, supernatants were incubated with Protein A/G magnetic beads (Pierce) at 4° C. for 2 hours. Beads were collected using a magnet and washed three times in NP40 lysis buffer. For western blot analysis, beads were resuspended in 1×LDS buffer containing 30 mM DTT and boiled at 95° C. for 5 minutes.

SDS PAGE and Immunoblotting

Equal amount of protein was separated on a 4-12% Bis-Tris SDS PAGE gel. Proteins were transferred onto a nitrocellulose membrane using a Trans-blot Turbo semi-dry transfer cell. Membranes were blocked in 5% milk powder in PBST for half an hour prior to incubation with primary antibody overnight at 4° C. or 1 hours at RT. Primary antibodies used in this study were: rabbit polyclonal anti-cyclin F (1:300; cat #sc-952, Santa Cruz Biotechnology), mouse monoclonal anti-mCherry (1:300; cat #632543, Clonetech) mouse monoclonal anti-TDP-43 (1:1000; cat #H00023435-M01, Abnova), mouse monoclonal β-tubulin (1:1000; cat #T5168, Sigma). After incubation, membranes were washed in PBS-T three times for 10 minutes before fluorescently labelled IRDye 800CW Goat Anti-Rabbit IgG (1:15,000; cat #926-32211, LI-COR) secondary antibody was applied for 30 minutes at RT. Proteins were imaged using a Li-Cor Odyssey imaging system at the appropriate wavelength.

Producing Recombinant Protein for Pull-Down Studies

Gene sequences encoding GST-tagged cyclin F were cloned into pGEX5 vectors. Constructs encoding the cyclin domain of cyclin F (aa 302-497) were also cloned into a pGEX5 vector. A MR/AA mutation was also introduced into the cyclin domain. This occurs in the hydrophobic patch (sequence MRYIL) at amino acids 309-313. All resulting constructs were individually transformed into Rosetta codon plus BL21 E. coli in order to generate recombinant protein. Prior to the induction of recombinant protein expression, 5 mL of LB broth (with ampicillin and chloramphenicol) was inoculated with a single colony of transformed rosetta E. coli BL21 and grown at 37° C. on an orbital shaker. After 17 hours, the starting culture was used to inoculate 400 mL of LB media (containing ampicillin and chloramphenicol). This culture was grown at 37° C. for 6-7 hours. Protein expression was induced with 0.13 mM IPTG overnight at 18° C. Harvested cells were lysed by sonication (Bioruptor) in ice-cold 2×PBS (pH7.4) containing 0.2% NP40 and protease inhibitor tablets (Roche). After centrifugation at 14,000×g, cleared lysates were incubated with GST sepharose matrix beads for (GE healthcare) for 45 minutes before the column was washed 5 times with ice cold 2×PBS. Purified protein was eluted using elution buffer (10 mM reduced glutathione in 50 mM tris (pH 8), 1 mM DTT). To remove glutathione, purified proteins were dialysed in dialysis buffer (50 mM Tris (pH 8), 150 mM NaCl, 1 mM DTT) overnight at 4° C.

Immunofluorescence Microscopy

HEK293, NSC-34 or Neuro2a cells were grown on coverslips, then transfected with appropriate constructs encoding either mCherry-cyclin F, HA-cyclin F, Flag-cyclin F, TDP-43-HA or empty vector using Lipofectamine 2000 (Invitrogen). After 24 hours, cells were fixed in 4% formaldehyde for 15 min, and washed in PBS. Cells were permeabilized using PBS containing 0.2% Triton X-100 for 10 minutes, then blocked using 1% BSA-PBST with 0.2M glycine for 30 mins. Permeabilized cells were incubated with 1:1000 anti-TDP-43 (ProteinTech), anti-myc or anti-HA overnight at 4° C. Samples were then incubated with species-specific 1:500 Alexa Fluor 488 or 647 and the nucleus was stained with Hoechst nuclear dye. Fluorescent images of mCherry-cyclin F and TDP-43 expressing cells were obtained using a Zeiss AxioImager microscope.

Microscale Thermophoresis (MST)

Recombinant His-tagged TDP-43 (a gift from Professor Julie Atkin's laboratory) was diluted to 200 nM in PBS-T buffer (137 mM NaCl, 2.5 mM KCl, 10 mM Na2HPO4, 2 mM KH2PO4, pH 7.4; 0.05% (v/v) Tween-20). The RED-tris-NTA dye was diluted in PBS-T to 100 nM. For labelling, the recombinant protein and diluted dye were mixed in a 1:1 volume ratio and incubated for 30 minutes at room temperature. Recombinant cyclin F-GST was also diluted in PBS-T buffer prior to analysis. Concentrations of cyclin F-GST (cyclin box) ranged from 0-22 μM. Before MST measurements were carried out, samples were centrifuged for 10 minutes at 4° C. at 14,000×g. All MST measurements were performed on a NanoTemper Monolith™NT.115 instrument using standard treated capillaries at room temperature. Final dye concentration of 25 nM yielded the fluorescence intensity of around 300 counts at a LED power of 50%. Accordingly, the MST power range was between 40-60% intensity with a laser-on time of 30 s and a laser-off time of 5 s. All data were analysed by MO Affinity Analysis Software.

Pull-Down Assay

Recombinant full-length cyclin F or recombinant cyclin box of cyclin F were incubated with recombinant full-length TDP-43 in ice-cold PBS at 4° C. for 4 hours whilst rotating. After this time, prewashed Ni-NTA magnetic beads were added to the mixture for 1 hour and left to incubate at 4° C. whilst rotating. Beads were then separated from the mixture and washed five times with PBS before beads were boiled in 2× Laemmli sample buffer (BioRad) at 95° C. for 5 minutes.

In Vitro Ubiquitylation Assay

HEK293 cells were transfected with cyclin F-Flag, enzymatically dead cyclin F (LP/AA) or an empty vector control using lipofectamine 2000 according to manufacturer's instructionumberingns. Cells were lysed in NP40 lysis buffer (50 mM Tris-HCl, 150 mM NaCl, 2 mM EDTA, 2 mM EGTA, 1% (v/v) NP-40, pH 7.4) containing complete protease inhibitor cocktail. Cyclin F was immunoprecipitated using anti-Flag M2 antibody (Sigma) conjugated to magnetic protein A/G beads (Pierce). Immunoprecipitated protein was washed four times in lysis buffer, then two times in ubiquitylation assay buffer (100 mM Tris-HCl, 10 mM MgCl2, 0.2 mM dithiothreitol pH 8). Ubiquitylation assays were performed in a volume of 50 μL containing 1 mM ATP, 5 nM E1 (UBA1), 100 nM E2 (UBE2D3) and, 2 μg of biotinylated ubiquitin and 5 μg of recombinant TDP-43.

AAV-Mediated CCNF Overexpression in Wildtype Mice

AAV9 encoding the wildtype human CCNF gene (fused with GFP) under the control of the neuron-specific synapsin promotor (AAV-CCNF) was stereotactically injected into the brain of newborn wildtype mice. This involved injection of 1 μL of AAV particles (1×10¹³ vg/mL) into 4 sites each bilaterally into the brain of cryo-anaesthetized mice. The experimental control was injection of AAV9 encoding GFP alone under the control of the synapsin promotor. The mice were housed in standard housing conditions for 8 months, at which time the mice were perfused with PBS and brains collected for SDS-PAGE and immunoblotting (as described above).

mRNA-Mediated CCNF Overexpression in TDP-43 Transgenic Zebrafish

Transgenic zebrafish expressing GFP-labeled human TDP-43 in motor neurons were used to assess CCNF interactions. Fluorescent (mKate) CCNF RNA (WT, S621G) was injected (˜2 nL) into the one-cell stage of zebrafish embryos. Successfully injected larvae were validated using the fluorescent reporter and raised at 28.5° C. till 3-5 days post fertilization. At day 3-5 confocal microscopy images of GFP-positive spinal neurons were captured using the same acquisition settings for all treatment groups. Maximum intensity projections were used to calculate TDP-43 fluorescence intensities of spinal motor neurons. The average ratios (nucleus versus whole-cell intensity) of TDP-43 levels for the CCNF injected groups were compared to uninjected controls. Four different fish for each treatment group were analyzed, at at least three different locations (random) within the spinal cord of each fish. Analysis was blinded.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the disclosure without limiting the disclosure to any one embodiment or specific collection of features. Those of skill in the art will therefore appreciate that, in light of the instant disclosure, various modifications and changes can be made in the particular embodiments exemplified without departing from the scope of the present disclosure. All such modifications and changes are intended to be included within the scope of the appended claims.

1. A method for enhancing survival of a neuron, the method comprising, consisting or consisting essentially of increasing the level of cyclin F in the neuron regardless of the neuron's level or activity of endogenous cyclin F, thereby enhancing survival of the neuron.

2-35. (canceled)


SEQUENCE LISTING (7 sequences)

SEQ ID NO: 1 - DNA, 4287 bp, Homo sapiens
ctgcgcctgcgcgagggctacgcgcgctccggccggggcgcgggcgcgctctcaggcgggctccggcggcagcgacgcgagcgcggcgatggggagcggcggcgtggtccactgtaggtgtgccaagtgtttctgttatcctacaaagcgaagaataaggaggaggccccgaaacctgaccatcttgagtctccccgaagatgtgctctttcacatcctgaaatggctttctgtagaggacatcctggccgtccgagctgtacactcccagctgaaggacctggtggacaaccacgccagtgtgtgggcatgtgccagcttccaggagctgtggccgtctccagggaacctgaagctctttgaaagggctgctgaaaaggggaatttcgaagctgctgtgaagctgggcatagcctacctctacaatgaaggcctgtctgtgtctgatgaggcccgcgcagaagtgaatggcctgaaggcctctcgcttcttcagtctcgctgagcggctgaatgtgggtgccgcacctttcatctggctcttcatccgccctccgtggtcggtgagcggaagctgctgcaaggccgtggttcacgagagcctcagggcagagtgccagctgcagaggactcacaaagcatccatattgcactgcttgggcagagtgctgagtctgttcgaggatgaggagaagcagcagcaggcccatgacctgtttgaggaggctgctcatcagggatgtctgaccagctcctacctcctctgggaaagcgacaggaggacagatgtgtcagatcctgggcgatgcctccacagcttccgaaaactcagggactacgctgccaaaggctgctgggaagcgcagctgtctttagccaaagcctgtgcaaatgcaaaccagcttggactggaggtgagagcttccagtgagatcgtctgccagctatttcaggcttcccaggctgtcagtaaacaacaagtcttctccgtgcagaagggactcaatgacacaatgaggtacattctgatcgactggctggtggaagttgccaccatgaaggacttcacaagcctgtgcctgcacctgaccgtggagtgtgtggaccggtacctgcggaggaggctggtgccgcggtacaggctccagctgctgggcatcgcctgcatggtcatctgcacccggtttatcagtaaagagatcctgaccatccgggaggccgtatggctcacggacaacacttacaagtacgaggacctggtgagaatgatgggcgagatcgtctccgccttggaagggaagattcgagtccccactgtggtggattacaaggaggtcctgctgacgctagtccctgtggagctgagaacccagcacctgtgcagcttcctctgcgagctctccctgctgcacaccagcctgtccgcctacgccccagcccgcctggctgccgcagccctgctcctggccagactgacgcacgggcagacacagccctggaccactcagctgtgggacctcaccggattctcctatgaagacctcattccctgcgtcttgagcctccataagaagtgcttccatgatgacgcccccaaggactacaggcaagtctctctgaccgccgtgaagcagcggtttgaggacaagcgctatggagaaatcagccaggaagaggtgctgagctacagccagttgtgtgctgcattaggagtgacacaagacagccccgaccccccgactttcctcagcacaggggagatccacgccttcctcagctctccctcggggcggagaaccaaacggaagcgggagaacagcctccaggaagacagaggcagcttcgttaccacccccactgcggagctgtccagccaggaggagacgctgctgggcagcttcctcgactggagcctggactgctgctctggctatgaaggcgaccaggagagtgagggcgagaaggagggcgacgtgacagctcccagcggcatcctcgatgtcaccgtggtctacctgaacccagaacagcattgctgccaggaatccagtgatgaggaggcttgtccagaggacaagggaccccaggacccacaggcactggcgctggacacccagatccctgcaacccctggacccaaacccctggtccgcaccagccgggagccagggaaggacgtcacgacctcagggtactcctccgtcagcaccgcaagtcccacaagctccgtggacggtggcttgggggccctgccccaacctacctcagtgctgtccctggacagtgactcgcacacacagccctgccaccatcaggccaggaagtcatgtttacagtgtcgtcccccaagtcccccggagagcagtgttccccagcaacaggtgaagcggataaacctatgcatacacagtgaggaggaggacatgaacctgggccttgtgaggctgtaagtgtgtcagcacatttgccgcagtggatgtgtactgagggggctggaggcgaagggtgggagcatagcataggaacgctgcatagaccatggaggcctttgcgcagagagcagagaggatgacttgcggccaccaagtttctgtctccgcgggagtcccgtgcaagccatcagaatgttgaaatgagggtgaagagctcagatccctctctttggaaagtttagcctggaagcagttggccacactgtgtggagggcacctctctgtcccttccgtgtctcactgtctctggaagcttcagcccatgtgtgtcctggtgttcccagccccaccagagccccgtgccgggagctgacagctttcacgcttaaggcacgtgtgacctgggtagtcagacaccacttgagcccctgcccacatctgctggtttggggcttcagtggggagctgacagctgtgagcacaccactgtcccctcatccacctcggcctgcatggggcacccacttccttctgggtggggcttccatggtaagggggcctgcgtccctgcacactgcgaggactgccttggccacaggcccactccctacgacacgtgactcgttttagagctctgtcccagaggcgttcgtatgtgacccacagatggcgtcaatgtgaacacctctctttgtgctgaatttctgggccattcttttcctgtcttatttctaaatttccttcttccaagatgaaaacaaaagaaaaacttaaaacagaaggtattaaaaaaacaagagattcccaccattatttaggttcacctgcaaaacaaaaatcttactccagcccctcaatgccatcctgacacactttatgcaaaaagaattttcccagataggctagccagaaaaaacttcaagtcctctgtaacatctgaggtgaccaagaggcagaagagcagagcagtcgggggccgtgtcctggctgatcccaactgcagctctgctgtgggggcccgtgggagggaggcagacccctgggctttcctgctggccacggagactctgctcctgcatggaaagggagcctgggagccagcagcccacgcctggggagcctgcctggggccatgtgaccatggcctctccctgggaacgggctgaccacaacacaccctgctgccatccacttctgtttactctgcaaatgtaagaaagaaccacttggccagaagtgtcccccagatgcttttttttttttttttttggagacagttttgctcttgtctccccggctggagtgcagtggcatgatctcaactctcaactcactgtaacctccgcctcccggatactcctgcctcagcctcctgggtagctgggattacaagcacccaaccacgcccagctaatttttgtattttcggtagagacgggatttcaccatgttggccaggctagtctcgaactcatgacctcaagtgatccgcccacttcggtctcccaaagtgctgggattacaggcatgagccacggcgcctggcccccaaatgctcttgaaccggaaacccagggatgggagatgctcactgagctgctgcttttatgtgtgctggtgctatgtgtgttcatgtccgcggcagctgtctttttgctactataagggaattctggccaccctgggtggggtgtggtcggggtgagaacccaagcgttggaactgtagacccgtcctgtcgactgtgtgcccctgggcatgtgtgagcctcagtttcctcatctgtaaggggggcaatgatacctacctcacaggggtgttgtgaggattaaatgtgaggaggatagtggcagatg

SEQ ID NO: 2 - DNA, 2361 bp, Homo sapiens
atggggagcggcggcgtggtccactgtaggtgtgccaagtgtttctgttatcctacaaagcgaagaataaggaggaggccccgaaacctgaccatcttgagtctccccgaagatgtgctctttcacatcctgaaatggctttctgtagaggacatcctggccgtccgagctgtacactcccagctgaaggacctggtggacaaccacgccagtgtgtgggcatgtgccagcttccaggagctgtggccgtctccagggaacctgaagctctttgaaagggctgctgaaaaggggaatttcgaagctgctgtgaagctgggcatagcctacctctacaatgaaggcctgtctgtgtctgatgaggcccgcgcagaagtgaatggcctgaaggcctctcgcttcttcagtctcgctgagcggctgaatgtgggtgccgcacctttcatctggctcttcatccgccctccgtggtcggtgagcggaagctgctgcaaggccgtggttcacgagagcctcagggcagagtgccagctgcagaggactcacaaagcatccatattgcactgcttgggcagagtgctgagtctgttcgaggatgaggagaagcagcagcaggcccatgacctgtttgaggaggctgctcatcagggatgtctgaccagctcctacctcctctgggaaagcgacaggaggacagatgtgtcagatcctgggcgatgcctccacagcttccgaaaactcagggactacgctgccaaaggctgctgggaagcgcagctgtctttagccaaagcctgtgcaaatgcaaaccagcttggactggaggtgagagcttccagtgagatcgtctgccagctatttcaggcttcccaggctgtcagtaaacaacaagtcttctccgtgcagaagggactcaatgacacaatgaggtacattctgatcgactggctggtggaagttgccaccatgaaggacttcacaagcctgtgcctgcacctgaccgtggagtgtgtggaccggtacctgcggaggaggctggtgccgcggtacaggctccagctgctgggcatcgcctgcatggtcatctgcacccggtttatcagtaaagagatcctgaccatccgggaggccgtatggctcacggacaacacttacaagtacgaggacctggtgagaatgatgggcgagatcgtctccgccttggaagggaagattcgagtccccactgtggtggattacaaggaggtcctgctgacgctagtccctgtggagctgagaacccagcacctgtgcagcttcctctgcgagctctccctgctgcacaccagcctgtccgcctacgccccagcccgcctggctgccgcagccctgctcctggccagactgacgcacgggcagacacagccctggaccactcagctgtgggacctcaccggattctcctatgaagacctcattccctgcgtcttgagcctccataagaagtgcttccatgatgacgcccccaaggactacaggcaagtctctctgaccgccgtgaagcagcggtttgaggacaagcgctatggagaaatcagccaggaagaggtgctgagctacagccagttgtgtgctgcattaggagtgacacaagacagccccgaccccccgactttcctcagcacaggggagatccacgccttcctcagctctccctcggggcggagaaccaaacggaagcgggagaacagcctccaggaagacagaggcagcttcgttaccacccccactgcggagctgtccagccaggaggagacgctgctgggcagcttcctcgactggagcctggactgctgctctggctatgaaggcgaccaggagagtgagggcgagaaggagggcgacgtgacagctcccagcggcatcctcgatgtcaccgtggtctacctgaacccagaacagcattgctgccaggaatccagtgatgaggaggcttgtccagaggacaagggaccccaggacccacaggcactggcgctggacacccagatccctgcaacccctggacccaaacccctggtccgcaccagccgggagccagggaaggacgtcacgacctcagggtactcctccgtcagcaccgcaagtcccacaagctccgtggacggtggcttgggggccctgccccaacctacctcagtgctgtccctggacagtgactcgcacacacagccctgccaccatcaggccaggaagtcatgtttacagtgtcgtcccccaagtcccccggagagcagtgttccccagcaacaggtgaagcggataaacctatgcatacacagtgaggaggaggacatgaacctgggccttgtgaggctgtaa

SEQ ID NO: 3 - protein, 786 aa, Homo sapiens
MGSGGVVHCRCAKCFCYPTKRRIRRRPRNLTILSLPEDVLFHILKWLSVEDILAVRAVHSQLKDLVDNHASVWACASFQELWPSPGNLKLFERAAEKGNFEAAVKLGIAYLYNEGLSVSDEARAEVNGLKASRFFSLAERLNVGAAPFIWLFIRPPWSVSGSCCKAVVHESLRAECQLQRTHKASILHCLGRVLSLFEDEEKQQQAHDLFEEAAHQGCLTSSYLLWESDRRTDVSDPGRCLHSFRKLRDYAAKGCWEAQLSLAKACANANQLGLEVRASSEIVCQLFQASQAVSKQQVFSVQKGLNDTMRYILIDWLVEVATMKDFTSLCLHLTVECVDRYLRRRLVPRYRLQLLGIACMVICTRFISKEILTIREAVWLTDNTYKYEDLVRMMGEIVSALEGKIRVPTVVDYKEVLLTLVPVELRTQHLCSFLCELSLLHTSLSAYAPARLAAAALLLARLTHGQTQPWTTQLWDLTGFSYEDLIPCVLSLHKKCFHDDAPKDYRQVSLTAVKQRFEDKRYGEISQEEVLSYSQLCAALGVTQDSPDPPTFLSTGEIHAFLSSPSGRRTKRKRENSLQEDRGSFVTTPTAELSSQEETLLGSFLDWSLDCCSGYEGDQESEGEKEGDVTAPSGILDVTVVYLNPEQHCCQESSDEEACPEDKGPQDPQALALDTQIPATPGPKPLVRTSREPGKDVTTSGYSSVSTASPTSSVDGGLGALPQPTSVLSLDSDSHTQPCHHQARKSCLQCRPPSPPESSVPQQQVKRINLCIHSEEEDMNLGLVRL

SEQ ID NO: 4 - DNA, 4196 bp, Homo sapiens
ggtctgcgcctgcgcgagggctacgcgcgctccggccggggcgcgggcgcgctctcaggcgggctccggcggcagcgacgcgagcgcggcgatggggagcggcggcgtggtccactgtaggtgtgccaagtgtttctgttatcctacaaagcgaagaataaggaggaggccccgaaacctgaccatcttgagtctccccgaagatgtgctctttcacatcctgaaatggctttctgtagaggacatcctggccgtccgagctggctgctgaaaaggggaatttcgaagctgctgtgaagctgggcatagcctacctctacaatgaaggcctgtctgtgtctgatgaggcccgcgcagaagtgaatggcctgaaggcctctcgcttcttcagtctcgctgagcggctgaatgtgggtgccgcacctttcatctggctcttcatccgccctccgtggtcggtgagcggaagctgctgcaaggccgtggttcacgagagcctcagggcagagtgccagctgcagaggactcacaaagcatccatattgcactgcttgggcagagtgctgagtctgttcgaggatgaggagaagcagcagcaggcccatgacctgtttgaggaggctgctcatcagggatgtctgaccagctcctacctcctctgggaaagcgacaggaggacagatgtgtcagatcctgggcgatgcctccacagcttccgaaaactcagggactacgctgccaaaggctgctgggaagcgcagctgtctttagccaaagcctgtgcaaatgcaaaccagcttggactggaggtgagagcttccagtgagatcgtctgccagctatttcaggcttcccaggctgtcagtaaacaacaagtcttctccgtgcagaagggactcaatgacacaatgaggtacattctgatcgactggctggtggaagttgccaccatgaaggacttcacaagcctgtgcctgcacctgaccgtggagtgtgtggaccggtacctgcggaggaggctggtgccgcggtacaggctccagctgctgggcatcgcctgcatggtcatctgcacccggtttatcagtaaagagatcctgaccatccgggaggccgtatggctcacggacaacacttacaagtacgaggacctggtgagaatgatgggcgagatcgtctccgccttggaagggaagattcgagtccccactgtggtggattacaaggaggtcctgctgacgctagtccctgtggagctgagaacccagcacctgtgcagcttcctctgcgagctctccctgctgcacaccagcctgtccgcctacgccccagcccgcctggctgccgcagccctgctcctggccagactgacgcacgggcagacacagccctggaccactcagctgtgggacctcaccggattctcctatgaagacctcattccctgcgtcttgagcctccataagaagtgcttccatgatgacgcccccaaggactacaggcaagtctctctgaccgccgtgaagcagcggtttgaggacaagcgctatggagaaatcagccaggaagaggtgctgagctacagccagttgtgtgctgcattaggagtgacacaagacagccccgaccccccgactttcctcagcacaggggagatccacgccttcctcagctctccctcggggcggagaaccaaacggaagcgggagaacagcctccaggaagacagaggcagcttcgttaccacccccactgcggagctgtccagccaggaggagacgctgctgggcagcttcctcgactggagcctggactgctgctctggctatgaaggcgaccaggagagtgagggcgagaaggagggcgacgtgacagctcccagcggcatcctcgatgtcaccgtggtctacctgaacccagaacagcattgctgccaggaatccagtgatgaggaggcttgtccagaggacaagggaccccaggacccacaggcactggcgctggacacccagatccctgcaacccctggacccaaacccctggtccgcaccagccgggagccagggaaggacgtcacgacctcagggtactcctccgtcagcaccgcaagtcccacaagctccgtggacggtggcttgggggccctgccccaacctacctcagtgctgtccctggacagtgactcgcacacacagccctgccaccatcaggccaggaagtcatgtttacagtgtcgtcccccaagtcccccggagagcagtgttccccagcaacaggtgaagcggataaacctatgcatacacagtgaggaggaggacatgaacctgggccttgtgaggctgtaagtgtgtcagcacatttgccgcagtggatgtgtactgagggggctggaggcgaagggtgggagcatagcataggaacgctgcatagaccatggaggcctttgcgcagagagcagagaggatgacttgcggccaccaagtttctgtctccgcgggagtcccgtgcaagccatcagaatgttgaaatgagggtgaagagctcagatccctctctttggaaagtttagcctggaagcagttggccacactgtgtggagggcacctctctgtcccttccgtgtctcactgtctctggaagcttcagcccatgtgtgtcctggtgttcccagccccaccagagccccgtgccgggagctgacagctttcacgcttaaggcacgtgtgacctgggtagtcagacaccacttgagcccctgcccacatctgctggtttggggcttcagtggggagctgacagctgtgagcacaccactgtcccctcatccacctcggcctgcatggggcacccacttccttctgggtggggcttccatggtaagggggcctgcgtccctgcacactgcgaggactgccttggccacaggcccactccctacgacacgtgactcgttttagagctctgtcccagaggcgttcgtatgtgacccacagatggcgtcaatgtgaacacctctctttgtgctgaatttctgggccattcttttcctgtcttatttctaaatttccttcttccaagatgaaaacaaaagaaaaacttaaaacagaaggtattaaaaaaacaagagattcccaccattatttaggttcacctgcaaaacaaaaatcttactccagcccctcaatgccatcctgacacactttatgcaaaaagaattttcccagataggctagccagaaaaaacttcaagtcctctgtaacatctgaggtgaccaagaggcagaagagcagagcagtcgggggccgtgtcctggctgatcccaactgcagctctgctgtgggggcccgtgggagggaggcagacccctgggctttcctgctggccacggagactctgctcctgcatggaaagggagcctgggagccagcagcccacgcctggggagcctgcctggggccatgtgaccatggcctctccctgggaacgggctgaccacaacacaccctgctgccatccacttctgtttactctgcaaatgtaagaaagaaccacttggccagaagtgtcccccagatgcttttttttttttttttttggagacagttttgctcttgtctccccggctggagtgcagtggcatgatctcaactctcaactcactgtaacctccgcctcccggatactcctgcctcagcctcctgggtagctgggattacaagcacccaaccacgcccagctaatttttgtattttcggtagagacgggatttcaccatgttggccaggctagtctcgaactcatgacctcaagtgatccgcccacttcggtctcccaaagtgctgggattacaggcatgagccacggcgcctggcccccaaatgctcttgaaccggaaacccagggatgggagatgctcactgagctgctgcttttatgtgtgctggtgctatgtgtgttcatgtccgcggcagctgtctttttgctactataagggaattctggccaccctgggtggggtgtggtcggggtgagaacccaagcgttggaactgtagacccgtcctgtcgactgtgtgcccctgggcatgtgtgagcctcagtttcctcatctgtaaggggggcaatgatacctacctcacaggggtgttgtgaggattaaatgtgaggaggatagtggcaaaaaaaaaaaaaaaaaa

SEQ ID NO: 5 - DNA, 1437 bp, Homo sapiens
atgaggtacattctgatcgactggctggtggaagttgccaccatgaaggacttcacaagcctgtgcctgcacctgaccgtggagtgtgtggaccggtacctgcggaggaggctggtgccgcggtacaggctccagctgctgggcatcgcctgcatggtcatctgcacccggtttatcagtaaagagatcctgaccatccgggaggccgtatggctcacggacaacacttacaagtacgaggacctggtgagaatgatgggcgagatcgtctccgccttggaagggaagattcgagtccccactgtggtggattacaaggaggtcctgctgacgctagtccctgtggagctgagaacccagcacctgtgcagcttcctctgcgagctctccctgctgcacaccagcctgtccgcctacgccccagcccgcctggctgccgcagccctgctcctggccagactgacgcacgggcagacacagccctggaccactcagctgtgggacctcaccggattctcctatgaagacctcattccctgcgtcttgagcctccataagaagtgcttccatgatgacgcccccaaggactacaggcaagtctctctgaccgccgtgaagcagcggtttgaggacaagcgctatggagaaatcagccaggaagaggtgctgagctacagccagttgtgtgctgcattaggagtgacacaagacagccccgaccccccgactttcctcagcacaggggagatccacgccttcctcagctctccctcggggcggagaaccaaacggaagcgggagaacagcctccaggaagacagaggcagcttcgttaccacccccactgcggagctgtccagccaggaggagacgctgctgggcagcttcctcgactggagcctggactgctgctctggctatgaaggcgaccaggagagtgagggcgagaaggagggcgacgtgacagctcccagcggcatcctcgatgtcaccgtggtctacctgaacccagaacagcattgctgccaggaatccagtgatgaggaggcttgtccagaggacaagggaccccaggacccacaggcactggcgctggacacccagatccctgcaacccctggacccaaacccctggtccgcaccagccgggagccagggaaggacgtcacgacctcagggtactcctccgtcagcaccgcaagtcccacaagctccgtggacggtggcttgggggccctgccccaacctacctcagtgctgtccctggacagtgactcgcacacacagccctgccaccatcaggccaggaagtcatgtttacagtgtcgtcccccaagtcccccggagagcagtgttccccagcaacaggtgaagcggataaacctatgcatacacagtgaggaggaggacatgaacctgggccttgtgaggctgtaa

SEQ ID NO: 6 - protein, 478 aa, Homo sapiens
MRYILIDWLVEVATMKDFTSLCLHLTVECVDRYLRRRLVPRYRLQLLGIACMVICTRFISKEILTIREAVWLTDNTYKYEDLVRMMGEIVSALEGKIRVPTVVDYKEVLLTLVPVELRTQHLCSFLCELSLLHTSLSAYAPARLAAAALLLARLTHGQTQPWTTQLWDLTGFSYEDLIPCVLSLHKKCFHDDAPKDYRQVSLTAVKQRFEDKRYGEISQEEVLSYSQLCAALGVTQDSPDPPTFLSTGEIHAFLSSPSGRRTKRKRENSLQEDRGSFVTTPTAELSSQEETLLGSFLDWSLDCCSGYEGDQESEGEKEGDVTAPSGILDVTVVYLNPEQHCCQESSDEEACPEDKGPQDPQALALDTQIPATPGPKPLVRTSREPGKDVTTSGYSSVSTASPTSSVDGGLGALPQPTSVLSLDSDSHTQPCHHQARKSCLQCRPPSPPESSVPQQQVKRINLCIHSEEEDMNLGLVRL

SEQ ID NO: 7 - protein, 786 aa, Artificial Sequence
  Synthetic sequence
MGSGGVVHCRCAKCFCYPTKRRIRRRPRNLTILSLPEDVLFHILKWLSVEDILAVRAVHSQLKDLVDNHASVWACASFQELWPSPGNLKLFERAAEKGNFEAAVKLGIAYLYNEGLSVSDEARAEVNGLKASRFFSLAERLNVGAAPFIWLFIRPPWSVSGSCCKAVVHESLRAECQLQRTHKASILHCLGRVLSLFEDEEKQQQAHDLFEEAAHQGCLTSSYLLWESDRRTDVSDPGRCLHSFRKLRDYAAKGCWEAQLSLAKACANANQLGLEVRASSEIVCQLFQASQAVSKQQVFSVQKGLNDTAAYILIDWLVEVATMKDFTSLCLHLTVECVDRYLRRRLVPRYRLQLLGIACMVICTRFISKEILTIREAVWLTDNTYKYEDLVRMMGEIVSALEGKIRVPTVVDYKEVLLTLVPVELRTQHLCSFLCELSLLHTSLSAYAPARLAAAALLLARLTHGQTQPWTTQLWDLTGFSYEDLIPCVLSLHKKCFHDDAPKDYRQVSLTAVKQRFEDKRYGEISQEEVLSYSQLCAALGVTQDSPDPPTFLSTGEIHAFLSSPSGRRTKRKRENSLQEDRGSFVTTPTAELSSQEETLLGSFLDWSLDCCSGYEGDQESEGEKEGDVTAPSGILDVTVVYLNPEQHCCQESSDEEACPEDKGPQDPQALALDTQIPATPGPKPLVRTSREPGKDVTTSGYSSVSTASPTSSVDGGLGALPQPTSVLSLDSDSHTQPCHHQARKSCLQCRPPSPPESSVPQQQVKRINLCIHSEEEDMNLGLVRL